SEQUENCE LISTING (106 sequences)

SEQ ID NO: 1 - DNA, 1368 bp, Ceriporiopsis subvermispora
atgaacgagaagatcctatctgccttctgcgtcatacttttctcattgtcggtcgccgctcgtcccaccgagaatggcccgcagatcgtcatagctaacaacgccggcacttatttgcccgttctacggggcagcggcaccaagtcttcctccgctgcggacgccacccaaacagtcccgttcgctagcgacgaccctaacccgcgtctatgggacattgataccaagaacctgacgaaggtcacgcctgagcgcggccagctcggtgctaagattctagggcctgacaacctcccgatcgacctccagaatgcagacacgcttgcgccacctacgaccgactcgggttcgattccgaatcccaagtggccgtttgctctcagccataacacactgtatagtggcggatgggtccgtattcagaacgacgaagtgatgccgattgcaaaagccatggccggagtgaacatgcgcttggaggctggcgcaatccgggagttgcactggcataataccccggagtgggcctatattctcaagggcacgactcaaattaccgcagtcgaccagaacgggcgaaactatctcgcgaacgttggacccggagacctgtggtacttccccgaaggcatgccccattcgcttcagggcacggacgcaaacaacgagggaagcgaattcttgttgatcttcccggacggaaccttcgattcatcgaatcaattcatgattaccgattggctagcacacacaccaaaggacgtcattgcgaagaacttcggcgtggatatctccgagtttgaccgcctcccttcgcacgatctgtacatcttcccgggagtcgcgccaccgctcgatgcgaaggccccggaggatcctcaaggaaccatccccctcccgtactcattcgagttctcgaaggttaagcctacgcagtacgccggtggtactgtcaagattgcggatacacggacgttcccgatcgcgaaaacgatcagcgtcgctgaggtaaccgtcgagcctggtgcaatgcgtgagctgcactggcatccgaccgaagatgaatggacgttcttcatcgagggccaagcacgcgtcaccatatttgctggccagagcaacgctcagacctacgactaccagggtggcgatatcgcgtacatacctactgcgtggggtcactatgttgaaaactccggtaatacgactctgcgtttcttggaaatcttcaactcccccttgtttgaggacgtcagtctggctcagtggattgccaacaccccgccggccattgtcaaagcaactctccagctctctgacgaagtcattaacacgctgaacaagagcaaggcctttgtcgtcggatag

SEQ ID NO: 2 - DNA, 1386 bp, Ceriporiopsis subvermispora
atgaacgagaagctcgtttctgtcttctgcgccatactggtcgcaatatccgtctctgctcgccccaccggcaacgatgtattctaccttccacgggccgttgcggtcagcagcgccggtgcctcttcgcccgcttcactgagtagcgggaccgaatcttcctctgcggcagaaccgacggagacggtgccgttcgcgagcgacgacccgaatccgcgtctatggaacatcgacacgcaggatctgtccgtagttgctcccgagcgcggcccgctgggagccaagatcattggacctgacaacctaccacttgatatccagaacgcagacacgcttgcgccaccgacgaccgactcgggttctatcccgaatgctaaatggccgttcgctctgagccacaatacgttgtacaccggcggatgggttcgcatacagaataacgaagtgctaccaatcgcgaaagccatggccggagtaaacatgcggttggaggctggtacaatccgggagctgcattggcacaacaccccagaatgggcctatatcctcaagggtacgactcagatcactgcggtcgatgagaatgggaaaaattatctcgcgaatgttggacctggagacttgtggtacttcccagagggcatgcctcattcactgcagggcacgaacgcaagtgacgagggaagcgagttcctgctgattttccctgacggcaccttcgatgcctcaaatcagttcatgattactgattggctggcacatacacccaaggacgttattgcgaaaaacttcggtgtggacatctccgaatttgatcgtcttccctcccacgacctatacatcttcccgggagttgcgccgccacttgatgctacagctcctgaggaccctcaaggcactatccccctcccgtactcattcgagttctcgaaagtggtgccaacacaatatgcagggggcactgtgaagattgcggacacacgcacattccctatctcgaagacaataagcgtcgccgagattaccgttgagccgggtgcgatgcgggagctacattggcaccccactgaagatgaatggactttcttcattgaggggcaggcacgcgtcaccctgttcgctggtgagagcaatgcacagacctacgactaccagggcggagatattgcctacattcccactgcgtatggtcactatgtcgaaaactccggtaacacgactttgcgtttcttggaaattttcaattctcccttgttccaggatgtcagtttgacccagtggcttgccaacactcctcgggctatagtcaaagcgactcttcagctttcggacaacgtcatcgactcattgaacaagagcaaggcattcgttgtcgcctcagattag

SEQ ID NO: 3 - DNA, 72 bp, Hordeum vulgare
atggccaacaagcacctgtccctctccctcttcctcgtgctcctcggcctctccgcctccctcgcctccgga

SEQ ID NO: 4 - DNA, 1308 bp, Artificial Sequence
  synthetic OXOX variant sequence
aggcctaccgaaaatggtccccaaatcgtgatagcaaataacgctggtacctacttgcctgtgcttcgtggctccggtacaaaatcctccagcgccgctgacgccactcaaactgtgcctttcgctagtgatgacccaaatccccgcttgtgggatattgatacaaaaaatttgacaaaagtcacacccgagcgcggtcagttgggtgctaagattttgggcccagataacttgcctattgacttgcaaaacgctgataccttggcaccacctaccactgattccgggtctattccaaatcccaagtggcctttcgcccttagtcataacactttgtactcaggaggctgggtccgtatacaaaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatcaaaatggccgtaattatttggcaaacgtgggaccaggggatctctggtattttcccgaagggatgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatttcctggggttgcccctccccttgacgctaaagcacccgaggaccctcagggtacaatacctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggaccttctttatcgagggacaggcacgtgtcactatttttgcaggccaaagtaatgcccaaacttacgactatcagggaggcgatatcgcttatatcccaaccgcctggggacactacgtagaaaattcagggaataccacattgcgcttcctggagatattcaattcccctctcttcgaggatgtctcccttgcacaatggatcgctaataccccaccagccatcgtcaaggctacacttcaattgtccgacgaggttatcaacacattgaataagtctaaggctttcgttgtaggttga

SEQ ID NO: 5 - DNA, 1326 bp, Artificial Sequence
  synthetic OXOX variant sequence
aggcctactggcaatgatgtcttttaccttccacgtgctgtagcagtttcctcagcaggagcatctagcccagcaagcttgtccagcggcactgaaagcagctccgcagcagagcccaccgaaaccgtgccattcgctagcgatgaccccaatcctcgcctttggaatattgacactcaggatctgagtgtagtggcccccgaacgcggacctttgggtgcaaagattatcgggccagataacttgcccctcgacattcaaaacgctgatacactcgctccacccacaaccgattctggatctattccaaacgcaaaatggccatttgctcttagccacaatacactgtacactggtggttgggttcgcatacaaaacaatgaagttttgccaattgccaaggctatggctggagtcaatatgcgtctggaggctggtactattcgcgaattgcactggcataatacacctgaatgggcctacattcttaaggggactacacagattaccgctgtagacgaaaacgggaagaattacttggccaacgttgggccaggagacctttggtacttccctgagggaatgccacacagccttcagggcactaatgcctccgacgagggatctgagttccttctgatatttcctgatggaacttttgatgcttccaaccagttcatgataactgattggcttgcccatacccccaaagacgttatcgcaaagaactttggagtggatataagcgaatttgatcgtctcccatcccacgatttgtatatatttcctggcgtggctcctccactcgatgcaacagctccagaagatccccagggaactatccccctcccttactcatttgaattctctaaagtcgtaccaacacagtatgctggcgggactgtgaagatcgctgacacccgtacctttcctatcagtaaaacaataagtgttgctgaaataacagttgaacctggtgctatgcgcgagctgcactggcatcctactgaggatgagtggactttcttcattgagggacaagctcgtgttacattgtttgcaggagagtccaacgctcaaacatatgactatcaaggaggggatatcgcatatataccaaccgcctatggacactatgtcgagaattctggaaatacaactctccgtttcttggagattttcaactcacctctgttccaggacgtgagtctcactcaatggctcgctaacaccccacgcgccattgtcaaggcaacactgcagcttagtgataatgtgatagacagtctcaataagtccaaggctttcgtcgtggcttctgattga

SEQ ID NO: 6 - DNA, 1308 bp, Artificial Sequence
  synthetic OXOX variant sequence
aggcctaccgaaaatggtccccaaatcgtgatagcaaataacgctggtacctacttgcctgtgcttcgtggctccggtacaaaatcctccagcgccgctgacgccactcaaactgtgcctttcgctagtgatgacccaaatccccgcttgtgggatattgatacaaaaaatttgattaaagtcacacccgagcgcggtcagttgggtgctaagattttgggcccagataacttgcctattgacttgcaaaacgctgataccttggcaccacctaccactgattccgggtctattccaaatcccaagtggcctttcgcccttagtcataacactttgtactcaggaggctgggtccgtatacaaaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatcaaaatggccgtaattatttggcaaacgtgggaccaggggatctctggtattttcccgaagggatgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatttcctggggttgcccctccccttgacgctaaagcacccgaggaccctcagggtacaatacctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggaccttctttatcgagggacaggcacgtgtcactatttttgcaggccaaagtaatgcccaaacttacgactatcagggaggcgatatcgcttatatcccaaccgcctggggacactacgtagaaaattcagggaatacccagttgcgcttcctggagatattcaattcccctctcttcgaggatgtctcccttgcacaatggatcgctaataccccaccagccatcgtcaaggctacacttcaattgtccgacgaggttatcaacacattgaataagcagaaggctttcgttgtaggttga

SEQ ID NO: 7 - DNA, 1308 bp, Artificial Sequence
  synthetic OXOX variant sequence
aggcctaccgaaaatggtccccaaatcgtgatagcaaataacgctggtacctacttgcctgtgcttcgtggctccggtacaaaatcctccagcgccgctgacgccactcaaactgtgcctttcgctagtgatgacccaaatccccgcttgtgggatattgatacaaaaaatttgaggaaagtcacacccgagcgcggtcagttgggtgctaagattttgggcccagataacttgcctattgacttgcaaaacgctgataccttggcaccacctaccactgattccgggtctattccaaatcccaagtggcctttcgcccttagtcataacactttgtactcaggaggctgggtccgtatacaaaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatcaaaatggccgtaattatttggcaaacgtgggaccaggggatctctggtattttcccgaagggatgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatttcctggggttgcccctccccttgacgctaaagcacccgaggaccctcagggtacaatacctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggaccttctttatcgagggacaggcacgtgtcactatttttgcaggccaaagtaatgcccaaacttacgactatcagggaggcgatatcgcttatatcccaaccgcctggggacactacgtagaaaattcagggaataccgtgttgcgcttcctggagatattcaattcccctctcttcgaggatgtctcccttgcacaatggatcgctaataccccaccagccatcgtcaaggctacacttcaattgtccgacgaggttatcaacacattgaataaggataaggctttcgttgtaggttga

SEQ ID NO: 8 - DNA, 1308 bp, Artificial Sequence
  synthetic OXOX variant sequence
aggcctaccgaaaatggtccccaaatcgtgatagcaaataacgctggtacctacttgcctgtgcttcgtggctccggtacaaaatcctccagcgccgctgacgccactcaaactgtgcctttcgctagtgatgacccaaatccccgcttgtgggatatggatacaaaaaatttgattaaagtcacacccgagcgcggtcagttgggtgctaagattttgggcccagataacttgcctattgacttgcaaaacgctgataccttggcaccacctaccactgattccgggtctgttccaaatcccaagtggcctttcgcccttagtcataacactttgtactcaggaggctacgtccgtatacaaaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatcaaaatggccgtaattatttggcaaacgtgggaccaggggatctctggtattttcccgaagggatgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatatcctggggttgcccctccccttgacgctaaagcacccgaggaccctcagggtacagtccctctcccttacagttttgagttcagtaaggttaagcctacccaatatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaacttacgactatcagggaggcgatatcgcttatatcccaaccgcctggggacactacgtagaaaattcagggaatacccagttgcgcttcctggagatattcaattcccctctctacgaggatgtctcccttgcacaatggatcgctaataccccaccagccatcgtcaaggctacacttcaattgtccgacgaggttatcaacacattgaataagcagaaggctttcgttgtaggttga

SEQ ID NO: 9 - DNA, 1308 bp, Artificial Sequence
  synthetic OXOX variant sequence
aggcctaccgaaaatggtccccaaatcgtgatagcaaataacgctggtacctacttgcctgtgcttcgtggctccggtacaaaatcctccagcgccgctgacgccactcaaactgtgcctttcgctagtgatgacccaaatccccgcttgtgggatattgatacaaaaaatttgattaaagtcacacccgagcgcggtcagttgggtgctaagattttgggcccagataacttgcctattgacttgcaaaacgctgataccttggcaccacctaccactgattccgggtctattccaaatcccaagtggcctttcgcccttagtcataacactttgtactcaggaggctacgtccgtatacaaaatgacgaggttatgcccattgctaaggctatggcaggtgttaatatgcgcctggaagcaggagcaatccgcgagctccattggcataatacaccagaatgggcctatatcttgaagggaactacccaagtcacagccgtagatcaaaatggccgtaattatttggcaaacgtgggaccaggggatctctggtattttcccgaagggatgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatatcctggggttgcccctccccttgacgctaaagcacccgaggaccctcagggtacagtccctctccctttcagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaacttacgactatcagggaggcgatatcgcttatatcccaaccgcctggggacactacgtagaaaattcagggaatacccagttgcgcttcctggagatattcaattcccctctctacgaggatgtctcccttgcacaatggatcgctaataccccaccagccatcgtcaaggctacacttcaattgtccgacgaggttatcaacacattgaataagcagaaggctttcgttgtaggttga

SEQ ID NO: 10 - DNA, 1308 bp, Artificial Sequence
  synthetic OXOX variant sequence
aggcctaccgaaaatggtccccaaatcgtgatagcaaataacgctggtacctacttgcctgtgcttcgtggctccggtacaaaatcctccagcgccgctgacgccactcaaactgtgcctttcgctagtgatgacccaaatccccgcttgtgggatatggatacaaaaaatttggagaaagtcacacccgagcgcggtcagttgggtgctaagattttgggcccagataacttgcctattgacttgcaaaacgctgataccttggcaccacctaccactgattccgggtctgttccaaatcccaagtggcctttcgcccttagtcataacactttgtactcaggaggctacgtccgtatacaaaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatcaaaatggccgtaattatttggcaaacgtgggaccaggggatctctggtattttcccgaagggatgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatatcctggggttgcccctccccttgacgctaaagcacccgaggaccctcagggtacagtccctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcagggaggcgatatcgcttatatcccaaccgcctggggacactacgtagaaaattcagggaatacccagttgcgcttcctggagatattcaattcccctctctacgaggatgtctcccttgcacaatggatcgctaataccccaccagccatcgtcaaggctacacttcaattgtccgacgaggttatcaacacattgaataagcagaaggctttcgttgtaggttga

SEQ ID NO: 11 - DNA, 1308 bp, Artificial Sequence
  synthetic OXOX variant sequence
aggcctaccgaaaatggtccccaaatcgtgatagcaaataacgctggtacctacttgcctgtgcctcgtggctccggtacaaaatcctccagcgccgctgacgccactcaaactgtgcctttcgctagtgatgacccaaatccccgcttgtgggatattgatacaaaaaatttggaaaaagtccaacccgagcgcggtcagttgggtgctaagattttgggcccagataacttgcctattgacttgcaaaacgctgataccttggcaccacctaccactgattccgggtctgttccaaatcccaagtggcctttcgcccttagtcataacactttgtactcaggaggctgggtccgtatacaaaatgacgaggttatgcccattgctaaggctatggcaggtgttaatatgcgcctggaagcaggtgcaatccgcgagctccattggcataatacaccagaatgggcctacatcttgaagggaactacccaaataacagccgttgatcaaaatggccgtaattatatggcaaacgtgggaccaggggatctctggtattttcccgaagggatgccacattcaatccaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatatcctggggttgcccctccccttgacgctaaagcacccgaggaccctcagggtacagtccctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaacttacgactatcagggaggcgatatcgcttatatcccaaccgcctggggacactacgtagaaaattcagggaatacccagttgcgctatctggagatattcaattcccctctctacgaggatgtctcccttgcacaatggatcgctaataccccaccagccatcgtcaaggctacacttcaattgtccgacgaggttatcaacacattgaataagcagaaggctttcgttgtaggttga

SEQ ID NO: 12 - DNA, 1308 bp, Artificial Sequence
  synthetic OXOX variant sequence
aggcctaccgaaaatggtccccaaatcgtgatagcaaataacgctggtacctacttgcctgtgcctcgtggctccggtacaaaatcctccagcgccgctgacgccactcaaactgtgcctttcgctagtgatgacccaaatccccgcttgtgggatatggatacaaaaaatttggagaaagtcacacccgagcgcggccagttgggtgctaagattttgggcccagataacttgcctattgacttgcaaaacgctgataccttggcaccacctaccactgattccgggtctgttccaaatcccaagtggcctttcgcccttagtcataacactttgtactcaggaggctacgtccgtatacaaaatgacgaggttatgcccattgctaaggctatggcaggtgttaatatgcgcctggaagcaggtgcaatccgcgagctccattggcataatacagcagaatgggcctatgttttgaagggaactacccaaataacagccgtagatcagaatggccgtaattatatggcaaacgtgggaccaggggatctctggtattttcccgaagggatgccacattcactgcaaggtaccgacgcaaataacgagggatctgaattcttgctgatatttccagacggaacttttaattctagcaaccagtttatgataactgattggttggctcacacccctaaggacgttattgccaagaatttcggtgtggacatttccgagttcgatcgtctgccatctcatgatctgtacatatatcctggggttgcccctccccttgacgctaaagcacccgaggaccctcagggtacagtccctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcaggccggcgatatcgcttatatcccaaccgcctggggacactacgtagaaaattcagggaatacccagttgcgcttcctggagatattcaattcccctctctacgaggatgtctcccttgcacaatggatcgctaataccccaccagccatcgtcaaggctacacttcaattgtccgacgaggttatcaacacactgaataagcagaaggctttcgttgtaggttga

SEQ ID NO: 13 - DNA, 1314 bp, Artificial Sequence
  synthetic OXOX variant sequence
atgaggcctaccgaaaatggtccccaaatcgtgatagcaaataacgctggtacctacttgcctgtgcttcgtggctccggtacaaaatcctccagcgccgctgacgccactcaaactgtgcctttcgctagtgatgacccaaatccccgcttgtggaatattgatacaaaaaatttggagaaagtcacaccccagcgcggtcagttgggtgctgagattttgggcccagataacttgcctattgacttgcaaaacgctgataccttggcaccacctaccactgattccgggtctattccaaatcccaagtggccttttgcccttagtcataacactttgtacactggaggctgggtccgtatacaaaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatgaaaatggcaaaaattatttggcaaacgtgggaccaggggatctctggtattttcccgaaggggtgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatttcctggggttgcccctccccttgacgctaaagcacccgaggaccctcagggtacagtccctctccctttcagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggaccttctttatcgagggacaggcacgtgtcactatttttgcaggccaaagtaatgcccaaactttcgactatcagggaggcgatatcgcttatatcccaaccgcctggggacactacgtagaaaattcagggaatacccaattgcgcttcctggagatattcaattcccctctcttcgaggatatttcccttgcacaatggatcgctaataccccaccagccatcgtcaaggctacacttcaattgtccgacgaggttatcaacacattgaataagcaaaaggctttcgttgtagcgtctgat

SEQ ID NO: 14 - DNA, 1308 bp, Artificial Sequence
  synthetic OXOX variant sequence
atgaggcctaccgaaaatggtccccaaatcgtgatagcaaataacgctggtacctacttgcctgtgcttcgtggctccggtacaaaatcctccagcgccgctgacgccactcaaactgtgcctttcgctagtgatgacccaaatccccgcttgtggaatattgatacacaggacttgtctgttgtcgcccccgagcgcggtcctttgggtgctaagattttgggcccagataacttgcctattgacttgcaaaacgctgataccttggcaccacctaccactgattccgggtctgttccaaatcccaagtggcctttcgcccttagtcataacactttgtactcaggaggctgggtccgtatacaaaataacgaggttctgcccattgctaaggctatggcaggtgttaatatgcgcctggaagcaggtgcaatccgcgagctccattggcataatacaccagaatgggcctatatcttgaagggaactacccaaataacagccgtagatcaaaatggccgtaattatttggcaaacgtgtctccaggggatctctggtattttcccgaagggatcccacattcactgcaaggtaccgacgcaaataacgagggaagcgaattcttgctgatatttccagacggaacttttgacgcgagcaaccagtttatgataactgattggttggctcacacccctaaagacgttattgccaagaatttcggtgtggacatttccgagttcgatcgtctgccatctcatgatctgtacatatttcctggggttgcccctccccttgacgctaaagcacccgaggaccctcagggtacagtccctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggaccttctttatcgagggacaggcacgtgtcactatttttgcaggccaaagtaatgcccaaacttacgactatcagggaggcgatatcgcttatatcccaaccgcctacggacactacgtagaaaattcagggaatacccagttgcgcttcctggagatattcaattcccctctcttcgaggatgtctcccttgcacaatggatcgctaataccccaccagccatcgtcaaggctacacttcaattgtccgacgaggttatcgatagcttgaataagcagaaggcttccgttgtaggt

SEQ ID NO: 15 - DNA, 1308 bp, Artificial Sequence
  synthetic OXOX variant sequence
atgaggcctaccgaaaatggtccccaaatcgtgatagcaaataacgctggtacccacttgcctgtgcctcgtggctccggtacaaaatcctccagcgccgctgacgccactcaaactgtgcctttcgctagtgatgacccaaatccccgcttgtgggatatggatacaaaaaatttggagaaagtcacacccgagcgcggtcagttgggtgctaagattttgggcccagataacttgcctattgacttgcaaaacgctgataccttggcaccacctaccactgattccgggtctgttccaaatcccaagtggcctttcgcccttagtcataacactttgtactcaggaggctacgtccgtatacaaaatgacgaggttatgcccattgctacggctatggcaggtgttaatatgcgcctggaagcaggtgcaatccgcgagctccattggcataatacaccagaatgggcctatatcttgaagggaactacccaaataacagccgtagatcaaaacggccgtaattatatggcaaacgtgggaccaggggatctctggtattttccccctgggatgccacattcactgcaaggtaccgacgcaaataacgagggaagcgaattcttgctgatatttccagacggaacttttgactctagcaaccagtttatgataactgattggttggctcacacccccaaagacgttattgccaagaatttcggtgtggacatttccgagttcgatcgtctgccgtctcatgatctgtacatatatcctggggttgcccctccccttgacgctcaggcacccgaggaccctcagggtacaatacctctcccttacagttttgagttcagtaaggttaagcctacccaatatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcagggaggcgatatcgcttatatcccaaccgcctggggacactacgtagaaaattcagggaatacccagttgcgcttcctggagatattcaattccgatgtgtacgaggatgtctcccttgcacaatggatcgctaataccccaccagccatcgtcaaggctacacttcaattgtccgacgaggttatcaacacattgaataagcagaaggctttcgttgtaggt

SEQ ID NO: 16 - DNA, 1308 bp, Artificial Sequence
  synthetic OXOX variant sequence
atgaggcctaccgaaaatggtccccaaatcgtgatagcaaataacgctggtacctacttgcctgtgcttcgtggctccggtacaaaatcctccagcgccgctgacgccactcaaactgtgcctttcgctagtgatgacccaaatccccgcttgtgggatatggatacaaaaaatttggaaaaagtccaacccgagcgcggtcagttgggtgctaagattttgggcccagataacttgcctattgacttgcaaaacgctgataccttggcaccacctaccactgattccgggtctattccaaaccccaagtggcctttctcccttagtcataacactttgtactcaggaggctacgtccgtatacaaaatgacgaggttatgcccattgctaaggctatggcaggtgttaatatgcgcctggaagcaggtgcaatccgtgagctccattggcataatacaccagaatgggcctatatcttgaagggaactacccaaataacagccgtagatcaaaatggccgtaattatttggcaaacgtgggaccaggggatctctggtattttcccgaagggatgccacattcaatccaaggtaccgacgcaaataacgagggaagcgaattcttgctgatatttccagacggaacttttgactccagcaaccagtttatgataactgattggttggctcacacccctaaagacgttatcgccaagaatttcggtgtggacatttccgagttcgatcgtctgccatctcatgatctgtacatatatcctggggttgcccctccccttgacgctaaagcacccgaggaccctcagggtacagtccctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcagggaggcgatatcgcttatatcccaaccgcctggggacactacgtagaaaattcagggaatacccagttgcgcttcctggagatattcaattcccctctctacgaggatgtctcccttgcacaatggatcgctaataccccaccagccatcgtcaaggctacacttcaattgtccgacgaggttatcaacacattgaataagcagaaggctttcgttgtaggt

SEQ ID NO: 17 - DNA, 1308 bp, Artificial Sequence
  synthetic OXOX variant sequence
atgaggcctaccgaaaatggtccccaaatcgtgatagcaaataacgctggtacctacttgcctgtgcctcgtggctccggtacaaaatcctccagcgccgctgacgccactcaaactgtgcctttcgctagtgatgacccaaatccccgcttgtgggatattgatacaaaaaatttgattaaagtcacgcccgagcgcggccagttgggtgctaagattttgggcccagataacttgcctattgacttgcaaaacgctgataccttggcaccacctaccactgattccgggtctgttccaaatcccaagtggcctttcgcccttagtcataacactttgtactcaggaggctacgtccgtatacaaaatgacgaggttatgcccattgctaaggctatggcaggtgttaatatgcgcctggaagcaggtgcaatccgcgagatgcattggcataatacaccagaatgggcctatgttttgaagggaactacccaaataacagccgtagatgagaatggccgtaattatatggcaaacgtgggaccaggggatctctggtattttcccgaagggatgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatatcctggggttgcccctccccttgacgctaaagcacccgaggaccctcagggtacagtccctctcccttacagttttgagttcagtaaggttaagcctacccaatatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcagggaggcgatatcgcttatatcccaaccgcctggggacactacgtagaaaattcagggaatacccagttgcgctatctggagatattcaattcccctctcttcgaggatgtctcccttgcacaatggatcgctaataccccaccagccatcgtcaaggctacacttcaattgtccgacgaggttatcaacacatttaataagcagaaggctttggttgtaggt

SEQ ID NO: 18 - protein, 455 aa, Ceriporiopsis subvermispora
MNEKILSAFCVILFSLSVAARPTENGPQIVIANNAGTYLPVLRGSGTKSSSAADATQTVPFASDDPNPRLWDIDTKNLTKVTPERGQLGAKILGPDNLPIDLQNADTLAPPTTDSGSIPNPKWPFALSHNTLYSGGWVRIQNDEVMPIAKAMAGVNMRLEAGAIRELHWHNTPEWAYILKGTTQITAVDQNGRNYLANVGPGDLWYFPEGMPHSLQGTDANNEGSEFLLIFPDGTFDSSNQFMITDWLAHTPKDVIAKNFGVDISEFDRLPSHDLYIFPGVAPPLDAKAPEDPQGTIPLPYSFEFSKVKPTQYAGGTVKIADTRTFPIAKTISVAEVTVEPGAMRELHWHPTEDEWTFFIEGQARVTIFAGQSNAQTYDYQGGDIAYIPTAWGHYVENSGNTTLRFLEIFNSPLFEDVSLAQWIANTPPAIVKATLQLSDEVINTLNKSKAFVVG

SEQ ID NO: 19 - protein, 461 aa, Ceriporiopsis subvermispora
MNEKLVSVFCAILVAISVSARPTGNDVFYLPRAVAVSSAGASSPASLSSGTESSSAAEPTETVPFASDDPNPRLWNIDTQDLSVVAPERGPLGAKIIGPDNLPLDIQNADTLAPPTTDSGSIPNAKWPFALSHNTLYTGGWVRIQNNEVLPIAKAMAGVNMRLEAGTIRELHWHNTPEWAYILKGTTQITAVDENGKNYLANVGPGDLWYFPEGMPHSLQGTNASDEGSEFLLIFPDGTFDASNQFMITDWLAHTPKDVIAKNFGVDISEFDRLPSHDLYIFPGVAPPLDATAPEDPQGTIPLPYSFEFSKVVPTQYAGGTVKIADTRTFPISKTISVAEITVEPGAMRELHWHPTEDEWTFFIEGQARVTLFAGESNAQTYDYQGGDIAYIPTAYGHYVENSGNTTLRFLEIFNSPLFQDVSLTQWLANTPRAIVKATLQLSDNVIDSLNKSKAFVVASD

SEQ ID NO: 20 - protein, 24 aa, Hordeum vulgare
MANKHLSLSLFLVLLGLSASLASG

SEQ ID NO: 21 - protein, 435 aa, Artificial Sequence
  synthetic OXOX variant sequence
RPTENGPQIVIANNAGTYLPVLRGSGTKSSSAADATQTVPFASDDPNPRLWDIDTKNLTKVTPERGQLGAKILGPDNLPIDLQNADTLAPPTTDSGSIPNPKWPFALSHNTLYSGGWVRIQNDEVMPIAKAMAGVNMRLEAGAIRELHWHNTPEWAYILKGTTQITAVDQNGRNYLANVGPGDLWYFPEGMPHSLQGTDANNEGSEFLLIFPDGTFDSSNQFMITDWLAHTPKDVIAKNFGVDISEFDRLPSHDLYIFPGVAPPLDAKAPEDPQGTIPLPYSFEFSKVKPTQYAGGTVKIADTRTFPIAKTISVAEVTVEPGAMRELHWHPTEDEWTFFIEGQARVTIFAGQSNAQTYDYQGGDIAYIPTAWGHYVENSGNTTLRFLEIFNSPLFEDVSLAQWIANTPPAIVKATLQLSDEVINTLNKSKAFVVG

SEQ ID NO: 22 - protein, 441 aa, Artificial Sequence
  synthetic OXOX variant sequence
RPTGNDVFYLPRAVAVSSAGASSPASLSSGTESSSAAEPTETVPFASDDPNPRLWNIDTQDLSVVAPERGPLGAKIIGPDNLPLDIQNADTLAPPTTDSGSIPNAKWPFALSHNTLYTGGWVRIQNNEVLPIAKAMAGVNMRLEAGTIRELHWHNTPEWAYILKGTTQITAVDENGKNYLANVGPGDLWYFPEGMPHSLQGTNASDEGSEFLLIFPDGTFDASNQFMITDWLAHTPKDVIAKNFGVDISEFDRLPSHDLYIFPGVAPPLDATAPEDPQGTIPLPYSFEFSKVVPTQYAGGTVKIADTRTFPISKTISVAEITVEPGAMRELHWHPTEDEWTFFIEGQARVTLFAGESNAQTYDYQGGDIAYIPTAYGHYVENSGNTTLRFLEIFNSPLFQDVSLTQWLANTPRAIVKATLQLSDNVIDSLNKSKAFVVASD

SEQ ID NO: 23 - protein, 435 aa, Artificial Sequence
  synthetic OXOX variant sequence
RPTENGPQIVIANNAGTYLPVLRGSGTKSSSAADATQTVPFASDDPNPRLWDIDTKNLIKVTPERGQLGAKILGPDNLPIDLQNADTLAPPTTDSGSIPNPKWPFALSHNTLYSGGWVRIQNDEVMPIAKAMAGVNMRLEAGAIRELHWHNTPEWAYILKGTTQITAVDQNGRNYLANVGPGDLWYFPEGMPHSLQGTDANNEGSEFLLIFPDGTFDSSNQFMITDWLAHTPKDVIAKNFGVDISEFDRLPSHDLYIFPGVAPPLDAKAPEDPQGTIPLPYSFEFSKVKPTQYAGGTVKIADTRTFPIAKTISVAEVTVEPGAMRELHWHPTEDEWTFFIEGQARVTIFAGQSNAQTYDYQGGDIAYIPTAWGHYVENSGNTQLRFLEIFNSPLFEDVSLAQWIANTPPAIVKATLQLSDEVINTLNKQKAFVVG

SEQ ID NO: 24 - protein, 435 aa, Artificial Sequence
  synthetic OXOX variant sequence
RPTENGPQIVIANNAGTYLPVLRGSGTKSSSAADATQTVPFASDDPNPRLWDIDTKNLRKVTPERGQLGAKILGPDNLPIDLQNADTLAPPTTDSGSIPNPKWPFALSHNTLYSGGWVRIQNDEVMPIAKAMAGVNMRLEAGAIRELHWHNTPEWAYILKGTTQITAVDQNGRNYLANVGPGDLWYFPEGMPHSLQGTDANNEGSEFLLIFPDGTFDSSNQFMITDWLAHTPKDVIAKNFGVDISEFDRLPSHDLYIFPGVAPPLDAKAPEDPQGTIPLPYSFEFSKVKPTQYAGGTVKIADTRTFPIAKTISVAEVTVEPGAMRELHWHPTEDEWTFFIEGQARVTIFAGQSNAQTYDYQGGDIAYIPTAWGHYVENSGNTVLRFLEIFNSPLFEDVSLAQWIANTPPAIVKATLQLSDEVINTLNKDKAFVVG

SEQ ID NO: 25 - protein, 435 aa, Artificial Sequence
  synthetic OXOX variant sequence
RPTENGPQIVIANNAGTYLPVLRGSGTKSSSAADATQTVPFASDDPNPRLWDMDTKNLIKVTPERGQLGAKILGPDNLPIDLQNADTLAPPTTDSGSVPNPKWPFALSHNTLYSGGYVRIQNDEVMPIAKAMAGVNMRLEAGAIRELHWHNTPEWAYILKGTTQITAVDQNGRNYLANVGPGDLWYFPEGMPHSLQGTDANNEGSEFLLIFPDGTFDSSNQFMITDWLAHTPKDVIAKNFGVDISEFDRLPSHDLYIYPGVAPPLDAKAPEDPQGTVPLPYSFEFSKVKPTQYAGGTVKIADTRTFPIAKTISVAEVTVEPGAMRELHWHPTEDEWTYFIEGQARVTIFAGQSNAQTYDYQGGDIAYIPTAWGHYVENSGNTQLRFLEIFNSPLYEDVSLAQWIANTPPAIVKATLQLSDEVINTLNKQKAFVVG

SEQ ID NO: 26 - protein, 435 aa, Artificial Sequence
  synthetic OXOX variant sequence
RPTENGPQIVIANNAGTYLPVLRGSGTKSSSAADATQTVPFASDDPNPRLWDIDTKNLIKVTPERGQLGAKILGPDNLPIDLQNADTLAPPTTDSGSIPNPKWPFALSHNTLYSGGYVRIQNDEVMPIAKAMAGVNMRLEAGAIRELHWHNTPEWAYILKGTTQVTAVDQNGRNYLANVGPGDLWYFPEGMPHSLQGTDANNEGSEFLLIFPDGTFDSSNQFMITDWLAHTPKDVIAKNFGVDISEFDRLPSHDLYIYPGVAPPLDAKAPEDPQGTVPLPFSFEFSKVKPTQYAGGTVKIADTRTFPIAKTISVAEVTVEPGAMRELHWHPTEDEWTYFIEGQARVTIFAGQSNAQTYDYQGGDIAYIPTAWGHYVENSGNTQLRFLEIFNSPLYEDVSLAQWIANTPPAIVKATLQLSDEVINTLNKQKAFVVG

SEQ ID NO: 27 - protein, 435 aa, Artificial Sequence
  synthetic OXOX variant sequence
RPTENGPQIVIANNAGTYLPVLRGSGTKSSSAADATQTVPFASDDPNPRLWDMDTKNLEKVTPERGQLGAKILGPDNLPIDLQNADTLAPPTTDSGSVPNPKWPFALSHNTLYSGGYVRIQNDEVMPIAKAMAGVNMRLEAGAIRELHWHNTPEWAYILKGTTQITAVDQNGRNYLANVGPGDLWYFPEGMPHSLQGTDANNEGSEFLLIFPDGTFDSSNQFMITDWLAHTPKDVIAKNFGVDISEFDRLPSHDLYIYPGVAPPLDAKAPEDPQGTVPLPYSFEFSKVKPTQYAGGTVKIADTRTFPIAKTISVAEVTVEPGAMRELHWHPTEDEWTYFIEGQARVTIFAGQSNAQTFDYQGGDIAYIPTAWGHYVENSGNTQLRFLEIFNSPLYEDVSLAQWIANTPPAIVKATLQLSDEVINTLNKQKAFVVG

SEQ ID NO: 28 - protein, 435 aa, Artificial Sequence
  synthetic OXOX variant sequence
RPTENGPQIVIANNAGTYLPVPRGSGTKSSSAADATQTVPFASDDPNPRLWDIDTKNLEKVQPERGQLGAKILGPDNLPIDLQNADTLAPPTTDSGSVPNPKWPFALSHNTLYSGGWVRIQNDEVMPIAKAMAGVNMRLEAGAIRELHWHNTPEWAYILKGTTQITAVDQNGRNYMANVGPGDLWYFPEGMPHSIQGTDANNEGSEFLLIFPDGTFDSSNQFMITDWLAHTPKDVIAKNFGVDISEFDRLPSHDLYIYPGVAPPLDAKAPEDPQGTVPLPYSFEFSKVKPTQYAGGTVKIADTRTFPIAKTISVAEVTVEPGAMRELHWHPTEDEWTYFIEGQARVTIFAGQSNAQTYDYQGGDIAYIPTAWGHYVENSGNTQLRYLEIFNSPLYEDVSLAQWIANTPPAIVKATLQLSDEVINTLNKQKAFVVG

SEQ ID NO: 29 - protein, 435 aa, Artificial Sequence
  synthetic OXOX variant sequence
RPTENGPQIVIANNAGTYLPVPRGSGTKSSSAADATQTVPFASDDPNPRLWDMDTKNLEKVTPERGQLGAKILGPDNLPIDLQNADTLAPPTTDSGSVPNPKWPFALSHNTLYSGGYVRIQNDEVMPIAKAMAGVNMRLEAGAIRELHWHNTAEWAYVLKGTTQITAVDQNGRNYMANVGPGDLWYFPEGMPHSLQGTDANNEGSEFLLIFPDGTFNSSNQFMITDWLAHTPKDVIAKNFGVDISEFDRLPSHDLYIYPGVAPPLDAKAPEDPQGTVPLPYSFEFSKVKPTQYAGGTVKIADTRTFPIAKTISVAEVTVEPGAMRELHWHPTEDEWTYFIEGQARVTIFAGQSNAQTFDYQAGDIAYIPTAWGHYVENSGNTQLRFLEIFNSPLYEDVSLAQWIANTPPAIVKATLQLSDEVINTLNKQKAFVVG

SEQ ID NO: 30 - protein, 437 aa, Artificial Sequence
  synthetic OXOX variant sequence
RPTENGPQIVIANNAGTYLPVLRGSGTKSSSAADATQTVPFASDDPNPRLWNIDTKNLEKVTPQRGQLGAEILGPDNLPIDLQNADTLAPPTTDSGSIPNPKWPFALSHNTLYTGGWVRIQNDEVMPIAKAMAGVNMRLEAGAIRELHWHNTPEWAYILKGTTQITAVDENGKNYLANVGPGDLWYFPEGVPHSLQGTDANNEGSEFLLIFPDGTFDSSNQFMITDWLAHTPKDVIAKNFGVDISEFDRLPSHDLYIFPGVAPPLDAKAPEDPQGTVPLPFSFEFSKVKPTQYAGGTVKIADTRTFPIAKTISVAEVTVEPGAMRELHWHPTEDEWTFFIEGQARVTIFAGQSNAQTFDYQGGDIAYIPTAWGHYVENSGNTQLRFLEIFNSPLFEDISLAQWIANTPPAIVKATLQLSDEVINTLNKQKAFVVASD

SEQ ID NO: 31 - protein, 435 aa, Artificial Sequence
  synthetic OXOX variant sequence
RPTENGPQIVIANNAGTYLPVLRGSGTKSSSAADATQTVPFASDDPNPRLWNIDTQDLSVVAPERGPLGAKILGPDNLPIDLQNADTLAPPTTDSGSVPNPKWPFALSHNTLYSGGWVRIQNNEVLPIAKAMAGVNMRLEAGAIRELHWHNTPEWAYILKGTTQITAVDQNGRNYLANVSPGDLWYFPEGIPHSLQGTDANNEGSEFLLIFPDGTFDASNQFMITDWLAHTPKDVIAKNFGVDISEFDRLPSHDLYIFPGVAPPLDAKAPEDPQGTVPLPYSFEFSKVKPTQYAGGTVKIADTRTFPIAKTISVAEVTVEPGAMRELHWHPTEDEWTFFIEGQARVTIFAGQSNAQTYDYQGGDIAYIPTAYGHYVENSGNTQLRFLEIFNSPLFEDVSLAQWIANTPPAIVKATLQLSDEVIDSLNKQKASVVG

SEQ ID NO: 32 - protein, 435 aa, Artificial Sequence
  synthetic OXOX variant sequence
RPTENGPQIVIANNAGTHLPVPRGSGTKSSSAADATQTVPFASDDPNPRLWDMDTKNLEKVTPERGQLGAKILGPDNLPIDLQNADTLAPPTTDSGSVPNPKWPFALSHNTLYSGGYVRIQNDEVMPIATAMAGVNMRLEAGAIRELHWHNTPEWAYILKGTTQITAVDQNGRNYMANVGPGDLWYFPPGMPHSLQGTDANNEGSEFLLIFPDGTFDSSNQFMITDWLAHTPKDVIAKNFGVDISEFDRLPSHDLYIYPGVAPPLDAQAPEDPQGTIPLPYSFEFSKVKPTQYAGGTVKIADTRTFPIAKTISVAEVTVEPGAMRELHWHPTEDEWTYFIEGQARVTIFAGQSNAQTFDYQGGDIAYIPTAWGHYVENSGNTQLRFLEIFNSDVYEDVSLAQWIANTPPAIVKATLQLSDEVINTLNKQKAFVVG

SEQ ID NO: 33 - protein, 435 aa, Artificial Sequence
  synthetic OXOX variant sequence
RPTENGPQIVIANNAGTYLPVLRGSGTKSSSAADATQTVPFASDDPNPRLWDMDTKNLEKVQPERGQLGAKILGPDNLPIDLQNADTLAPPTTDSGSIPNPKWPFSLSHNTLYSGGYVRIQNDEVMPIAKAMAGVNMRLEAGAIRELHWHNTPEWAYILKGTTQITAVDQNGRNYLANVGPGDLWYFPEGMPHSIQGTDANNEGSEFLLIFPDGTFDSSNQFMITDWLAHTPKDVIAKNFGVDISEFDRLPSHDLYIYPGVAPPLDAKAPEDPQGTVPLPYSFEFSKVKPTQYAGGTVKIADTRTFPIAKTISVAEVTVEPGAMRELHWHPTEDEWTYFIEGQARVTIFAGQSNAQTFDYQGGDIAYIPTAWGHYVENSGNTQLRFLEIFNSPLYEDVSLAQWIANTPPAIVKATLQLSDEVINTLNKQKAFVVG

SEQ ID NO: 34 - protein, 435 aa, Artificial Sequence
  synthetic OXOX variant sequence
RPTENGPQIVIANNAGTYLPVPRGSGTKSSSAADATQTVPFASDDPNPRLWDIDTKNLIKVTPERGQLGAKILGPDNLPIDLQNADTLAPPTTDSGSVPNPKWPFALSHNTLYSGGYVRIQNDEVMPIAKAMAGVNMRLEAGAIREMHWHNTPEWAYVLKGTTQITAVDENGRNYMANVGPGDLWYFPEGMPHSLQGTDANNEGSEFLLIFPDGTFDSSNQFMITDWLAHTPKDVIAKNFGVDISEFDRLPSHDLYIYPGVAPPLDAKAPEDPQGTVPLPYSFEFSKVKPTQYAGGTVKIADTRTFPIAKTISVAEVTVEPGAMRELHWHPTEDEWTYFIEGQARVTIFAGQSNAQTFDYQGGDIAYIPTAWGHYVENSGNTQLRYLEIFNSPLFEDVSLAQWIANTPPAIVKATLQLSDEVINTFNKQKALVVG

SEQ ID NO: 35 - protein, 436 aa, Artificial Sequence
  synthetic OXOX variant sequence
MRPTENGPQIVIANNAGTYLPVLRGSGTKSSSAADATQTVPFASDDPNPRLWDIDTKNLIKVTPERGQLGAKILGPDNLPIDLQNADTLAPPTTDSGSIPNPKWPFALSHNTLYSGGWVRIQNDEVMPIAKAMAGVNMRLEAGAIRELHWHNTPEWAYILKGTTQITAVDQNGRNYLANVGPGDLWYFPEGMPHSLQGTDANNEGSEFLLIFPDGTFDSSNQFMITDWLAHTPKDVIAKNFGVDISEFDRLPSHDLYIFPGVAPPLDAKAPEDPQGTIPLPYSFEFSKVKPTQYAGGTVKIADTRTFPIAKTISVAEVTVEPGAMRELHWHPTEDEWTFFIEGQARVTIFAGQSNAQTYDYQGGDIAYIPTAWGHYVENSGNTQLRFLEIFNSPLFEDVSLAQWIANTPPAIVKATLQLSDEVINTLNKQKAFVVG

SEQ ID NO: 36 - protein, 436 aa, Artificial Sequence
  synthetic OXOX variant sequence
MRPTENGPQIVIANNAGTYLPVLRGSGTKSSSAADATQTVPFASDDPNPRLWDIDTKNLRKVTPERGQLGAKILGPDNLPIDLQNADTLAPPTTDSGSIPNPKWPFALSHNTLYSGGWVRIQNDEVMPIAKAMAGVNMRLEAGAIRELHWHNTPEWAYILKGTTQITAVDQNGRNYLANVGPGDLWYFPEGMPHSLQGTDANNEGSEFLLIFPDGTFDSSNQFMITDWLAHTPKDVIAKNFGVDISEFDRLPSHDLYIFPGVAPPLDAKAPEDPQGTIPLPYSFEFSKVKPTQYAGGTVKIADTRTFPIAKTISVAEVTVEPGAMRELHWHPTEDEWTFFIEGQARVTIFAGQSNAQTYDYQGGDIAYIPTAWGHYVENSGNTVLRFLEIFNSPLFEDVSLAQWIANTPPAIVKATLQLSDEVINTLNKDKAFVVG

SEQ ID NO: 37 - protein, 436 aa, Artificial Sequence
  synthetic OXOX variant sequence
MRPTENGPQIVIANNAGTYLPVLRGSGTKSSSAADATQTVPFASDDPNPRLWDIDTKNLTKVTPERGQLGAKILGPDNLPIDLQNADTLAPPTTDSGSIPNPKWPFALSHNTLYSGGWVRIQNDEVMPIAKAMAGVNMRLEAGAIRELHWHNTPEWAYILKGTTQITAVDQNGRNYLANVGPGDLWYFPEGMPHSLQGTDANNEGSEFLLIFPDGTFDSSNQFMITDWLAHTPKDVIAKNFGVDISEFDRLPSHDLYIFPGVAPPLDAKAPEDPQGTIPLPYSFEFSKVKPTQYAGGTVKIADTRTFPIAKTISVAEVTVEPGAMRELHWHPTEDEWTFFIEGQARVTIFAGQSNAQTYDYQGGDIAYIPTAWGHYVENSGNTTLRFLEIFNSPLFEDVSLAQWIANTPPAIVKATLQLSDEVINTLNKSKAFVVG

SEQ ID NO: 38 - DNA, 10 bp, Hordeum vulgare
tcatcttctt

SEQ ID NO: 39 - DNA, 1308 bp, Artificial Sequence
  synthetic OXOX variant sequence
aggcctaccgaaaatggtccccaagtcgtgatagcaaataacgctggtacctacttgcctgtgcttcgtggctccggtacaaaatcctccagcgccgctgacgccactcaaactgtgcctttcgctagtgatgacccaaatccccgcttgtgggatattgatacaaaaaatttggagaaagtcacacccgagcgcggtcagttgggtgctaagattttgggcccagataacttgcctattgacttgcaaaacgctgataccttggcaccacctaccactgattccgggtctattccaaatcccaagtggcctttcgcccttagtcataacactttgtactcaggaggctgggtccgtatacaaaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatcaaaatggccgtaattatttggcaaacgtgggaccaggggatctctggtattttcccgaagggatgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatatcctggggttgcccctccccttgacgctaaagcacccgaggaccctcagggtacagtccctctcccttacagttttgagttcagtaaggctaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcagggaggcgatatcgcttatatcccaaccgcctggggacactacgtagaaaattcagggaatacccagttgcgcttcctggagatattcaattcccctctctacgaggatgtctcccttgcacaatggatcgctaataccccaccagccatcgtcaaggctacacttcaattgtccgacgaggttatcaacacattgaataagcagaaggctttcgttgtaggttga

SEQ ID NO: 40 - DNA, 1308 bp, Artificial Sequence
  synthetic OXOX variant sequence
aggcctaccgaaaatggtccccaaatcgtgatagcaaataacgctggtacctacttgcctgtgcttcgtggctccggtacaaaatcctccagcgccgctgacgccactcaaactgtgcctttcgctagtgatgacccaaatccccgcttgtggggtattgatacaaaaaatttggagaaagtcacacccgagcgcggtcagttgggtgctaagattttgggcccagataacttgcctattgacttgcaaaacgctgataccttggcaccacctaccactgattccgggtctgttccaaatcccaagtggcctttcgcccttagtcataacactttgtactcaggaggctgggtccgtatacagaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatcaaaatggccgtaattatttggcaaacgtgggaccaggggatctctggtattttcccgaagggatgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatatcctggggttgcccctccccttgacgctaaagcacccgaggaccctcagggtacagtccctctccctttcagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcagggaggcgatatcgcttatatcccaaccgcctggggacactacgtagaaaattcagggaatacccagttgcgcttcctggagatattcaattcccctctctacgaggatgtctcccttgcacaatggatcgctaataccccaccagccatcgtcaaggctacacttcaattgtccgacgaggttatcaacacattgaataagcagaaggctttcgttgtaggttga

SEQ ID NO: 41 - DNA, 1308 bp, Artificial Sequence
  synthetic OXOX variant sequence
aggcctaccgaaaatggtccccaaaccgtgatagcaaataacgctggtacctacttgtctgtgcttcgtggctccggtacaaaatcctccagcgccgctgacgccactcaaactgtgcctttcgctagtgatgacccaaatccccgcttgtgggatattgatacaaaaaatttggagaaagtcacacccgagcgcggtcagttgggtgctaagattttgggcccagataacttgcctattgacttgcaaaacgctgataccttggcaccacctaccactgattccgggtctgttccaaatcccaagtggcctttcgcccttagtcataacactttgtactcaggaggctgggtccgtatacaaaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatcaaaatggccgtaattatttggcaaacgtgggaccaggggatctctggtattttcccgaagggatgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatatcctggggttgcccctccccttgacgctaaagcacccgaggaccctcagggtacagtccctctccctttcagttttgagttcagtaaggttaagcctacccaatatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcagggaggcgatatcgcttatatcccaaccgcctggggacactacgtagaaaattcagggaatacccagttgcgcttcctggagatattcaattcccctctctacgaggatgtctcccttgcacaatggatcgctaataccccaccagccatcgtcaaggctacacttcaattgtccgacgaggttatcaacacattgaataagcagaaggctttcgttgtaggttga

SEQ ID NO: 42 - DNA, 1308 bp, Artificial Sequence
  synthetic OXOX variant sequence
aggcctaccgaaaatggtccccaaatcgtgatagcaaataacgctggtacctacttgcctgtgcttcgtggctccggtacaaaatcctccagcgccgctgacgccactcaaactgtgcctttcgctagtgatgacccaaatccccgcttgtgggatattgatacaaaaaatttggagaaagtcacacccgagcgcggtcagttgggtgctaagattttgggcccagataacttgcctattgacttgcaaaacgctgataccttggcaccacctaccactgattccgggtctgttccaaatcccaagtggcctttcgcccttagtcataacactttgtactcaggaggctgggtccgtatacaaaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatcaaaatggccgtaattatttggcaaacgtgggaccaggggatctctggtattttcccgaagggatgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatatcctggggttgcccctccccttgacgctaaagcgcccgaggaccctcagggtacagtccctctccctttcagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcagggaggcgatatcgcttatatcccaaccgcctggggacactacgtagaaaattcagggaatacccagttgcgcttcctggagatattcaattcccctctctacgaggatgtctcccttgcacaatggatcgctaataccccaccagccatcgtcaaggctacacttcaattgtccgacgaggttatcaacacattgaataagcagaaggctttcgttgtaggttga

SEQ ID NO: 43 - DNA, 1308 bp, Artificial Sequence
  synthetic OXOX variant sequence
aggcctaccgaaaatggtccccaaatcgtgatagcaaataacgctggtacctacttgcctgtgcttcgtggctccggtacaaaatcctccagcgccgctgacgccactcaaactgtgcctttcgctagtgatgacccaaatccccgcttgtgggatattgatacaaaaaatttggagaaagtcacacccgagcgcggtcagttgggtgctgagattttgggcccagataacatccctattgacttgcaaaacgctgataccttggcaccacctaccactgattccgggtctattccaaatcccaagtggcctttcgcccttagtcataacactttgtactcaggaggctacgtccgtatacaaaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatcaaaatggccgtaattatttggcaaacgtgggaccaggggatctctggtattttcccgaagggatcccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatttcctggggttgcccctccccttgacgctaaagcacccgaggaccctcagggtacagtccctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaacttacgactatcagggaggcgatatcgcttatatcccaaccgcctggggacactacgtagaaaattcagggaatacccagttgcgcttcctggagatattcaattcccctctctacgaggatgtctcccttgcacaatggatcgctaataccccaccagccatcgtcaaggctacacttcaattgtccgacgaggttatcaacacattgaataagcagaaggcttccgttgtaggttga

SEQ ID NO: 44 - DNA, 1308 bp, Artificial Sequence
  synthetic OXOX variant sequence
aggcctaccgaaaatggtccccaaatcgtgatagcaaataacgctggtacccacttgcctgtgcctcgtggctccggtacaaaatcctccagcgccgctgacgccactcaaactgtgcctttcgctagtgatgacccaaatccccgcttgtgggatattgatacacaggacttgtctgttgtcgcccccgagcgcggtcctttgggtgctaagattttgggcccagataacttgcctattgacttgcaaaacgctgataccttggcaccacctaccactgattccgggtctattccaaaccccaagtggcctttcgcccttagtcataacactttgtactcaggaggctacgtccgtatacaaaatgacgaggttatgcccattgctacggctatggcaggtgttaatatgcgcctggaagcaggtgcaatccgcgagctccattggcataatacaccagaatgggcctatatcttgaagggaactacccaaataacagccgtagatcaaaacggccgtaattatatggcaaacgtgggaccaggggatctctggtattttccccctgggatgccacattcactgcaaggtaccgacgcaaataacgagggaagcgaattcttgctgatatttccagacggaacttttgacgcgagcaaccagtttatgataactgattggttggctcacacccctaaagacgttattgccaagaatttcggtgtggacacttccgagttcgatcgtctgccgtctcatgatctgtacatatttcctggggttgcccctccccttgacgctcaggcacccgaggaccctcagggtacagtccctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcagggaggcgatatcgcttatatcccaaccgcctggggacactacgtagaaaattcagggaatacccagttgcgcttcctggagatattcaattccgatgtgtacgaggatgtctcccttgcacaatggatcgctaataccccaccagccatcgtcaaggctacacttcaattgtccgacgaggttatcaacacactgaataagcagaaggctttcgttgtaggttga

SEQ ID NO: 45 - DNA, 1308 bp, Artificial Sequence
  synthetic OXOX variant sequence
aggcctaccgaaaatggtccccaaatcgtgatagcaaataacgctggtacccacttgcctgtgcctcgtggctccggtacaaaatcctccagcgccgctgacgccactcaaactgtgcctttcgctagtgatgacccaaatccccgcttgtgggatattgatacaaaaaatttggagaaagtcacacccgagcgcggtcagttgggtgctaagattttgggcccagataacttgcctattgacttgcaaaacgctgataccttggcaccacctaccactgattccgggtctgttccaaatcccaagtggcctttcgcccttagtcataacactttgtactcaggaggctacgtccgtatacaaaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatcaaaacggccgtaattatatggcaaacgtgtctccaggggatctctggtattttccccctgggatgccacattcactgcaaggtaccgacgcaaataacgagggaagcgaattcttgctgatatttccagacggaacttttgactctagcaaccagtttatgataactgattggttggctcacacccccaaagacgttattgccaagaatttcggtgtggacatttccgagttcgatcgtctgccatctcatgatctgtacatatttcctggggttgcccctccccttgacgctaaagcacccgaggaccctcagggtacagtccctctcccttacagttttgagttcagtaaggttaagcctacccaatatgccggtggtactgttaaaatagctgatactcgtaccttccccatcgctaagaccatttctgttgctgaggttaccgtagaacctggagctatgcgcgagcttcactggcatcctactgaggatgagtggaccttctttatcgagggacaggcacgtgtcactatttttgcaggccaaagtaatgcccaaactttcgactatcagggaggcgatatcgcttatatcccaaccgcctacggacactacgtagaaaattcagggaatacccagttgcgcttcctggagatattcaattccgatgtgtacgaggatgtctcccttgcacaatggatcgctaataccccaccagccatcgtcaaggctacacttcaattgtccgacgaggttatcgatagcttgaataagcagaaggcttccgttgtaggttga

SEQ ID NO: 46 - DNA, 1314 bp, Artificial Sequence
  synthetic OXOX variant sequence
aggcctaccgaaaatggtccccaaatcgtgatagcaaataacgctggtacctacttgcctgtgcctcgtggctccggtacaaaatcctccagcgccgctgacgccactcaaactgtgcctttcgctagtgatgacccaaatccccgcttgtggaatattgatacacaggacttgtctgttgtcgcccccgagcgcggtcctttgggtgctaagattttgggcccagataacttgcctattgacttgcaaaacgctgataccttggcaccacctaccactgattccgggtctgttccaaatcccaagtggcctttcgcccttagtcataacactttgtactcaggaggctacgtccgtatacaaaatgacgaggttatgcccattgctaaggctatggcaggtgttaatatgcgcctggaagcaggtgcaatccgcgagatgcattggcataatacaccagaatgggcctatatcttgaagggaactacccaaataacagccgtagatcaaaatggccgtaattatttggcaaacgtgggaccaggggatctctggtattttccccctgggatgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatttcctggggttgcccctccccttgacgctaaagcacccgaggaccctcagggtacagtccctctcccttacagttttgagttcagtaaggttaagcctacccaatatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcagggaggcgatatcgcttatatcccaaccgcctggggacactacgtagaaaattcagggaatacccagttgcgcttcctggagatattcaattcccctctctacgaggatgtctcccttgcacaatggatcgctaataccccaccagccatcgtcaaggctacacttcaattgtccgacgaggttatcaacacactgaataagcaaaaggctttcgttgtagcgtctgattga

SEQ ID NO: 47 - DNA, 1314 bp, Artificial Sequence
  synthetic OXOX variant sequence
aggcctaccgaaaatggtccccaaatcgtgatagcaaataacgctggtacccacttgcctgtgcctcgtggctccggtacaaaatcctccagcgccgctgacgccactcaaactgtgcctttcgctagtgatgacccaaatccccgcttgtgggatatggatacaaaaaatttggagaaagtcacacccgagcgcggccagttgggtgctaagattttgggcccagataacttgcctattgacttgcaaaacgctgataccttggcaccacctaccactgattccgggtctgttccaaatcccaagtggcctttcgcccttagtcataacactttgtactcaggaggctacgtccgtatacaaaatgacgaggttctgcccattgctacggctatggcaggtgttaatatgcgcctggaagcaggtgcaatccgcgagctccattggcataatacaccagaatgggcctatatcttgaagggaactacccaaataacagccgtagatcagaatggccgtaattatatggcaaacgtgggaccaggggatctctggtattttccccctgggatgccacattcactgcaaggtaccgacgcaaataacgagggaagcgaattcctgctgatatttccagacggaacttttgactctagcaaccagtttatgataactgattggttggctcacacccctaaagacgttattgccaagaatttcggtgtggacatttccgagttcgatcgtctgccgtctcatgatctgtacatatttcctggggttgcccctccccttgacgctaaagcacccgaggaccctcagggtacagtccctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggaccttctttatcgagggacaggcacgtgtcactatttttgcaggccaaagtaatgcccaaactttcgactatcaggccggcgatatcgcttatatcccaaccgcctggggacactacgtagaaaattcagggaatacccagttgcgcttcctggagatattcaattcccctctctacgaggatgtctcccttgcacaatggatcgctaataccccaccagccatcgtcaaggctacacttcaattgtccgacgaggttatcaacacactgaataagcagaaggctttcgttgtagcgtctgattga

SEQ ID NO: 48 - DNA, 1308 bp, Artificial Sequence
  synthetic OXOX variant sequence
aggcctaccgaaaatggtccccaaatcgtgatagcaaataacgctggtacctacttgcctgtgcttcgtggctccggtacaaaatcctccagcgccgctgacgccactcaaactgtgcctttcgctagtgatgacccaaatccccgcttgtgggatatggatacaaaaaatttggagaaagtcacacccgagcgcggtcctttgggtgctaagattttgggcccagataacttgcctattgacttgcaaaacgctgataccttggcaccacctaccactgattccgggtctgttccaaatcccaagtggcctttcgcccttagtcataacactttgtactcaggaggctacgtccgtatacaaaatgacgaggttatgcccattgctaaggctatggcaggtgttaatatgcgcctggaagcaggtgcaatccgcgagatgcattggcataatacaccagaatgggcctatgttttgaagggaactacccaaataacagccgtagatcaaaacggccgtaattatttggcaaacgtgggaccaggggatctctggtattttcccgaaggggtgccacattcactgcaaggtaccgacgcaaataacgagggaagcgaattcttgctgatatttccagacggaacttttgactctagcaaccagtttatgataactgattggttggctcacacccccaaagacgttattgccaagaatttcggtgtggacatttccgagttcgatcgtctgccatctcatgatctgtacatatttcctggggttgcccctccccttgacgctaaagcacccgaggaccctcagggtacagtccctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcaggccggcgatatcgcttatatcccaaccgcctggggacactacgtagaaaattcagggaatacccaattgcgcttcctggagatattcaattcccctctcttcgaggatgtctcccttgcacaatggatcgctaataccccaccagccatcgtcaaggctacacttcaattgtccgacgaggttatcaacacattgaataagcagaaggcttccgttgtaggttga

SEQ ID NO: 49 - DNA, 1308 bp, Artificial Sequence
  synthetic OXOX variant sequence
aggcctaccgaaaatggtccccaaatcgtgatagcaaataacgctggtacccacttgcctgtgcctcgtggctccggtacaaaatcctccagcgccgctgacgccactcaaactgtgcctttcgctagtgatgacccaaatccccgcttgtgggatatggatacaaaaaatttggagaaagtcacgcccgagcgcggccagttgggtgctaagattttgggcccagataacttgcctattgacttgcaaaacgctgataccttggcaccacctaccactgattccgggtctgttccaaatcccaagtggcctttcgcccttagtcataacactttgtactcaggaggctgggtccgtatacaaaatgacgaggttatgcccattgctaaggctatggcaggtgttaatatgcgcctggaagcaggtgcaatccgcgagatgcattggcataatacaccagaatgggcctatgttttgaagggaactacccaaataacagccgtagatgagaatggccgtaattatatggcaaacgtgggaccaggggatctctggtattttccccctgggatgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatttcctggggttgcccctccccttgacgctaaagcacccgaggaccctcagggtacagtccctctccctttcagttttgagttcagtaaggttaagcctacccaatatgccggtggtactgttaaaatagctgatactcgtaccttccccatcgctaagaccatttctgttgctgaggttaccgtagaacctggagctatgcgcgagcttcactggcatcctactgaggatgagtggaccttctttatcgagggacaggcacgtgtcactatttttgcaggccaaagtaatgcccaaacttacgactatcagggaggcgatatcgcttatatcccaaccgcctggggacactacgtagaaaattcagggaatacccagttgcgcttcctggagatattcaattccgatgtgtacgaggatgtctcccttgcacaatggatcgctaataccccaccagccatcgtcaaggctacacttcaattgtccgacgaggttatcaacacactgaataagcagaaggcttccgttgtaggttga

SEQ ID NO: 50 - DNA, 1314 bp, Artificial Sequence
  synthetic OXOX variant sequence
aggcctaccgaaaatggtccccaaatcgtgatagcaaataacgctggtacctacttgcctgtgcttcgtggctccggtacaaaatcctccagcgccgctgacgccactcaaactgtgcctttcgctagtgatgacccaaatccccgcttgtgggatatggatacaaaaaatttggagaaagtcacacccgagcgcggccagttgggtgctaagattttgggcccagataacttgcctattgacttgcaaaacgctgataccttggcaccacctaccactgattccgggtctattccaaaccccaagtggcctttcgcccttagtcataacactttgtactcaggaggctacgtccgtatacaaaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatcaaaacggccgtaattatatggcaaacgtgtctccaggggatctctggtattttcccgaagggatgccacattcactgcaaggtaccgacgcaaataacgagggaagcgaattcttgctgatatttccagacggaacttttgactccagcaaccagtttatgataactgattggttggctcacacccctaaagacgttattgccaagaatttcggtgtggacatttccgagttcgatcgtctgccatctcatgatctgtacatatttcctggggttgcccctccccttgacgctaaagcacccgaggacccccagggtacagtccctctccctttcagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcaggccggcgatatcgcttatatcccaaccgcctggggacactacgtagaaaattcagggaatacccagttgcgcttcctggagatattcaattcccctctctacgaggatgtctcccttgcacaatggatcgctaataccccaccagccatcgtcaaggctacacttcaattgtccgacgaggttatcgatagcttgaataagcaaaaggctttcgttgtagcgtctgattga

SEQ ID NO: 51 - DNA, 1308 bp, Artificial Sequence
  synthetic OXOX variant sequence
aggcctaccgaaaatggtccccaaatcgtgatagcaaataacgctggtacctacttgcctgtgcttcgtggctccggtacaaaatcctccagcgccgctgacgccactcaaactgtgcctttcgctagtgatgacccaaatccccgcttgtgggatattgatacacaggacttgtctgttgtcgcccccgagcgcggtcctttgggtgctaagattttgggcccagataacttgcctattgacttgcaaaacgctgataccttggcaccacctaccactgattccgggtctgttccaaatcccaagtggcctttcgcccttagtcataacactttgtactcaggaggctacgtccgtatacaaaataacgaggttctgcccattgctaaggctatggcaggtgttaatatgcgcctggaagcaggtgcaatccgcgagctccattggcataatacaccagaatgggcctatgttttgaagggaactacccaaataacagccgtagatgagaatggccgtaattatatggcaaacgtgggaccaggggatctctggtattttcccgaagggatcccacattcactgcaaggtaccgacgcaaataacgagggaagcgaattcttgctgatatttccagacggaacttttgactccagcaaccagtttatgataactgattggttggctcacacccctaaggacgttattgccaagaatttcggtgtggacatttccgagttcgatcgtctgccatctcatgatctgtacatatttcctggggttgcccctccccttgacgctcaggcacccgaggaccctcagggtacagtccctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcagggaggcgatatcgcttatatcccaaccgcctggggacactacgtagaaaattcagggaatacccagttgcgcttcctggagatattcaattcccctctcttcgaggatgtctcccttgcacaatggatcgctaataccccaccagccatcgtcaaggctacacttcaattgtccgacgaggttatcgatagcttgaataagcagaaggcttccgttgtaggttga

SEQ ID NO: 52 - DNA, 1314 bp, Artificial Sequence
  synthetic OXOX variant sequence
aggcctaccgaaaatggtccccaaatcgtgatagcaaataacgctggtacctacttgcctgtgcctcgtggctccggtacaaaatcctccagcgccgccgacgccactcaaactgtgcctttcgctagtgatgacccaaatccccgcttgtggaatattgatacaaaaaatttgattaaagtcacgcccgagcgcggccagttgggtgctaagattttgggcccagataacttgcctattgacttgcaaaacgctgataccttggcaccacctaccactgattccgggtctattccaaaccccaagtggccttttgcccttagtcataacactttgtacactggaggctgggtccgtatacaaaatgacgaggttctgcccattgctaaggctatggcaggtgttaatatgcgcctggaagcaggtgcaatccgcgagatgcattggcataatacaccagaatgggcctatatcttgaagggaactacccaaataacagccgtagatgaaaatggccgtaattatatggcaaacgtgggaccaggggatctctggtattttccccctgggatgccacattcactgcaaggtaccgacgcaaataacgagggaagcgaattcttgctgatatttccagacggaacttttgactctagcaaccagtttatgataactgattggttggctcacacccctaaggacgttattgccaagaatttcggtgtggacatttccgagttcgatcgtctgccatctcatgatctgtacatatatcctggggttgcccctccccttgacgctcaggcacccgaggaccctcagggtacagtccctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcagggaggcgatatcgcttatatcccaaccgcctggggacactacgtagaaaattcagggaatacccagttgcgcttcctggagatattcaattcccctctcttcgaggatgtctcccttgcacaatggatcgctaataccccaccagccatcgtcaaggctacacttcaattgtccgacgaggttatcaacacatttaataagcagaaggctttcgttgtagcgtctgattga

SEQ ID NO: 53 - DNA, 1308 bp, Artificial Sequence
  synthetic OXOX variant sequence
aggcctaccgaaaatggtccccaaatcgtgatagcaaataacgctggtacctacttgcctgtgcctcgtggctccggtacaaaatcctccagcgccgctgacgccactcaaactgtgcctttcgctagtgatgacccaaatccccgcttgtgggatatggatacaaaaaatttggagaaggtcacaccccagcgcggtcagttgggtgctgagattttgggcccagataacttgcctattgacttgcaaaacgctgataccttggcaccacctaccactgattccgggtctgttccaaatcccaagtggcctttcgcccttagtcataacactttgtactcaggaggctgggtccgtatacaaaatgacgaggttatgcccattgctacggctatggcaggtgttaatatgcgcctggaagcaggtgcaatccgcgagctccattggcataatacaccagaatgggcctatatcttgaagggaactacccaaataacagccgtagatgaaaatggcaaaaattatttggcaaacgtgggaccaggggatctctggtattttcccgaaggggtgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatttcctggggttgcccctccccttgacgctcaggcacccgaggaccctcagggtacagtccctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcagggaggcgatatcgcttatatcccaaccgcctggggacactacgtagaaaattcagggaatacccagttgcgcttcctggagatattcaattccgatgtgtacgaggatgtctcccttgcacaatggatcgctaataccccaccagccatcgtcaaggctacacttcaattgtccgacgaggttatcaacacactgaataagcagaaggctttcgttgtaggttga

SEQ ID NO: 54 - DNA, 1308 bp, Artificial Sequence
  synthetic OXOX variant sequence
aggcctaccgaaaatggtccccaaatcgtgatagcaaataacgctggtacctacttgcctgtgcttcgtggctccggtacaaaatcctccagcgccgctgacgccactcaaactgtgcctttcgctagtgatgacccaaatccccgcttgtggaatattgatacacaggacttgtctgttgtcgcccccgagcgcggtcctttgggtgctaagattttgggcccagataacttgcctattgacttgcaaaacgctgataccttggcaccacctaccactgattccgggtctgttccaaatcccaagtggcctttcgcccttagtcataacactttgtactcaggaggctacgtccgtatacaaaatgacgaggttatgcccattgctaaggctatggcaggtgttaatatgcgcctggaagcaggtgcaatccgcgagatgcattggcataatacaccagaatgggcctatatcttgaagggaactacccaaataacagccgtagatcaaaacggccgtaattatatggcaaacgtgggaccaggggatctctggtattttcccgaagggatgccacattcactgcaaggtaccgacgcaaataacgagggaagcgaattcttgctgatatttccagacggaacttttgactctagcaaccagtttatgataactgattggttggctcacacccctaaagacgttattgccaagaacttcggtgtggacatttccgagttcgatcgtctgccatctcatgatctgtacatatttcctggggttgcccctccccttgacgctaaagcacccgaggaccctcagggtacagtccctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggaccttctttatcgagggacaggcacgtgtcactatttttgcaggccaaagtaatgcccaaactttcgactatcaggccggcgatatcgcttatatcccaaccgcctggggacactacgtagaaaattcagggaatacccagttgcgcttcctggagatattcaattcccctctcttcgaggatgtctcccttgcacaatggatcgctaataccccaccagccatcgtcaaggctacacttcaattgtccgacgaggttatcaacacactgaataagcagaaggctttcgttgtaggttga

SEQ ID NO: 55 - DNA, 1308 bp, Artificial Sequence
  synthetic OXOX variant sequence
aggcctaccgaaaatggtccccaaatcgtgatagcaaataacgctggtacccacttgcctgtgcctcgtggctccggtacaaaatcctccagcgccgctgacgccactcaaactgtgcctttcgctagtgatgacccaaatccccgcttgtgggatatggatacaaaaaatttggagaaagtcacacccgagcgcggccagttgggtgctgagattttgggcccagataacttgcctattgacctgcaaaacgctgataccttggcaccacctaccactgattccgggtctattccaaatcccaagtggccttttgcccttagtcataacactttgtactcaggaggctacgtccgtatacaaaatgacgaggttatgcccattgctaaggctatggcaggtgttaatatgcgcctggaagcaggtgcaatccgcgagatgcattggcataatacacaagaatgggcctatatcttgaagggaactacccaaataacagccgtagatcaaaatggccgtaattatttggcaaacgtgggaccaggggatctctggtattttcccgaagggatgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccgtctcatgatctgtacatatttcctggggttgcccctccccttgacgctaaagcacccgaggaccctcagggtacagtccctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcaggccggcgatatcgcttatatcccaaccgcctggggacactacgtagaaaattcagggaatacccagttgcgcttcctggagatattcaattcccctctctacgaggatgtctcccttgcacaatggatcgctaataccccaccagccatcgtcaaggctacacttcaattgtccgacgaggttatcaacacactgaataagcagaaggctttcgttgtaggttga

SEQ ID NO: 56 - DNA, 1308 bp, Artificial Sequence
  synthetic OXOX variant sequence
aggcctaccgaaaatggtccccaaatcgtgatagcaaataacgctggtacccacttgcctgtgcctcgtggctccggtacaaaatcctccagcgccgctgacgccactcaaactgtgcctttcgctagtgatgacccaaatccccgcttgtgggatatggatacaaaaaatttggagaaagtcacgccccagcgcggtcagttgggtgctgagattttgggcccagataacttgcctattgacttgcaaaacgctgataccttggcaccacctaccactgattccgggtctattccaaatcccaagtggccttttgcccttagtcataacactttgtactcaggaggctacgtccgtatacaaaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatcaaaatggccgtaattatttggcaaacgtgtctccaggggatctctggtattttcccgaagggatgccacattcactgcaaggtaccgacgcaaataacgagggatctgaattcttgctgatatttccagacggaacttttgactctagcaaccagtttatgataactgattggttggctcacacccctaaagacgttattgccaagaatttcggtgtggacatttccgagttcgatcgtctgccgtctcatgatctgtacatatttcctggggttgcccctccccttgacgctaaagcacccgaggaccctcagggtacagtccctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcaggccggcgatatcgcttatatcccaaccgcctggggacactacgtagaaaattcagggaatacccagttgcgcttcctggagatattcaattcccctctctacgaggatgtctcccttgcacaatggatcgctaataccccaccagccatcgtcaaggctacacttcaattgtccgacgaggttatcaacacactgaataagcagaaggctttcgttgtaggttga

SEQ ID NO: 57 - DNA, 1314 bp, Artificial Sequence
  synthetic OXOX variant sequence
aggcctaccgaaaatggtccccaaatcgtgatagcaaataacgctggtacctacttgcctgtgcctcgtggctccggtacaaaatcctccagcgccgctgacgccactcaaactgcgcctttcgctagtgatgacccaaatccccgcttgtgggatatggatacaaaaaatttggaaaaagtccaacccgagcgcggtcagttgggtgctaagattttgggcccagataacttgcctattgacttgcaaaacgctgataccttggcaccacctaccactgattccgggtctattccaaaccccaagtggcctttcgcccttagtcataacactttgtactcaggaggctacgtccgtatacaaaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatcaaaacggccgtaattatatggcaaacgtgggaccaggggatctctggtattttccccctgggatgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccgtctcatgatctgtacatatttcctggggttgcccctccccttgacgctcaggcacccgaggaccctcagggtacagtccctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaacttacgactatcagggaggcgatatcgcttatatcccaaccgcctggggacactacgtagaaaattcagggaatacccagttgcgcttcctggagatattcaattcccctctcttcgaggatgtctcccttgcacaatggatcgctaataccccaccagccatcgtcaaggctacacttcaattgtccgacgaggttatcaacacatttaataagcagaaggctttcgttgtagcgtctgattga

SEQ ID NO: 58 - DNA, 1308 bp, Artificial Sequence
  synthetic OXOX variant sequence
aggcctaccgaaaatggtccccaaatcgtgatagcaaataacgctggtacctacttgcctgtgcttcgtggctccggtacaaaatcctccagcgccgctgacgccactcaagctgtgcctttcgctagtgatgacccaaatccccgcttgtgggatatggatacaaaaaatttggagaaagtcacacccgagcgcggccagttgggtgctgagattttgggcccagataacttgcctattgacttgcaaaacgctgataccttggcaccacctaccactgattccgggtctgttccaaatcccaagtggccttttgcccttagtcataacactttgtactcaggaggctgggtccgtatacaaaataacgaggttctgcccattgctaaggctatggcaggtgttaatatgcgcctggaagcaggtgcaatccgcgagatgcattggcataatacaccagaatgggcctatatcttgaagggaactacccaaataacagccgtagatcaaaacggcaaaaattatttggcaaacgtgggaccaggggatctctggtattttccccctgggatgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatttcctggggttgcccctccccttgacgctgaagcacccgaggaccctcagggtacagtccctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggaccttctttatcgagggacaggcacgtgtcactatttttgcaggccaaagtaatgcccaaactttcgactatcagggaggcgatatcgcttatatcccaaccgcctggggacactacgtagaaaattcagggaatacccagttgcgcttcctggagatattcaattcccctctcttcgaggatgtctcccttgcacaatggatcgctaataccccaccagccatcgtcaaggctacacttcaattgtccgacgaggttatcgatagcttgaataagcagaaggctttcgttgtaggttga

SEQ ID NO: 59 - DNA, 1308 bp, Artificial Sequence
  synthetic OXOX variant sequence
aggcctaccgaaaatggtccccaaatcgtgatagcaaataacgctggtacctacttgcctgtgcctcgtggctccggtacaaaatcctccagcgccgctgacgccactcaaactgtgcctttcgctagtgatgacccaaatccccgcttgtgggatatggatacaaaaaatttggagaaagtcacacccgagcgcggtcagttgggtgctaagattttgggcccagataacttgcctattgacttgcaaaacgctgataccttggcaccacctaccactgattccgggtctattccaaatcccaagtggcctttcgcccttagtcataacactttgtactcaggaggctacgtccgtatacaaaatgacgaggttatgcccattgctaaggctatggcaggtgttaatatgcgcctggaagcaggtgcaatccgcgagatgcattggcataatacaccagaatgggcctatatcttgaagggaactacccaaataacagccgtagatcaaaacggccgtaattatatggcaaacgtgggaccaggggatctctggtattttcccgaagggatgccacattcactgcaaggtaccgacgcaaataacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatatcctggggttgcccctccccttgacgctaaagcacccgaggaccctcagggtacagtccctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaacttacgactatcagggaggcgatatcgcttatatcccaaccgcctggggacactacgtagaaaattcagggaatacccagttgcgcttcctggagatattcaattcccctctcttcgaggatgtctcccttgcacaatggatcgctaataccccaccagccatcgtcaaggctacacttcaattgtccgacgaggttatcaacacactgaataagcagaaggctttcgttgtaggttga

SEQ ID NO: 60 - DNA, 1308 bp, Artificial Sequence
  synthetic OXOX variant sequence
aggcctaccgaaaatggtccccaaatcgtgatagcaaataacgctggtacccacttgcctgtgcctcgtggctccggtacaaaatcctccagcgccgctgacgccactcaaactgtgcctttcgctagtgatgacccaaatccccgcttgtgggatatggatacaaaaaatttggagaaagtcacacccgagcgcggccagttgggtgctaagattttgggcccagataacttgcctattgacttgcaaaacgctgataccttggcaccacctaccactgattccgggtctattccaaatcccaagtggcctttcgcccttagtcataacactttgtactcaggaggctacgtccgtatacaaaatgacgaggttatgcccattgctacggctatggcaggtgttaatatgcgcctggaagcaggtgcaatccgcgagctccattggcataatacaccagaatgggcctatgttttgaagggaactacccaaataacagccgtagatcagaatggccgtaattatatggcaaacgtgggaccaggggatctttggtattttcccgaaggggtgccacattcactgcaaggtaccgacgcaaataacgagggatctgaattcttgctgatatttccagacggaacttttgactctagcaaccagtttatgataactgattggttggctcacacccccaaagacgttattgccaagaatttcggtgtggacatttccgagttcgatcgtctgccatctcatgatctgtacatatatcctggggttgcccctccccttgacgctaaagcacccgaggaccctcagggtacagtccctctcccttacagttttgagttctgtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggaccttctttatcgagggacaggcacgtgtcactatttttgcaggccaaagtaatgcccaaactttcgactatcagggaggcgatatcgcttatatcccaaccgcctggggacactacgtagaaaattcagggaatacccagttgcgcttcctggagatattcaattcccctctcttcgaggatgtctcccttgcacaatggatcgctaataccccaccagccatcgtcaaggctacacttcaattgtccgacgaggttatcaacacactgaataagcagaaggctttcgttgtaggttga

SEQ ID NO: 61 - DNA, 1308 bp, Artificial Sequence
  synthetic OXOX variant sequence
aggcctaccgaaaatggtccccaaatcgtgatagcaaataacgctggtacccacttgcctgtgcctcgtggcaccggtacaaaatcctccagcgccgctgacgccactcaaactgtgcctttcgctagtgatgacccaaatccccgcttgtgggatatggatacaaaaaatttggagaaagtcacacccgagcgcggccagttgggtgctaagattttgggcccagataacttgcctattgacttgcaaaacgctgataccttggcaccacctaccactgattccgggtctattccaaatcccaagtggcctttcgcccttagtcataacactttgtactcaggaggctgggtccgtatacaaaataacgaggttctgcccattgctacggctatggcaggtgttaatatgcgcctggaagcaggtgcaatccgcgagctccattggcataatacaccagaatgggcctatgttttgaagggaactacccaaataacagccgtagatcaaaacggccgtaattatatggcaaacgtgggaccaggggatctctggtattttccccctgggatgccacattcactgcaaggtaccgacgcaaataacgagggatctgaattcttgctgatatttccagacggaacttttgacgcgagcaaccagtttatgataactgattggttggctcacacccctaaagacgttattgccaagaatttcggtgtggacatttccgagttcgatcgtctgccatctcatgatctgtacatatttcctggggttgcccctccccttgacgctcaggcacccgaggaccctcagggtacagtccctctcccttacagttttgagttcagtaaggttaagcctacccaatatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcagggaggcgatatcgcttatatcccaaccgcctggggacactacgtagaaaattcagggaatacccagttgcgctatctggagatattcaattcccctctcttcgaggatatttcccttgcacaatggatcgctaataccccaccagccatcgtcaaggctacacttcaattgtccgacgaggttatcaacacactgaataagcagaaggcttccgttgtaggttga

SEQ ID NO: 62 - DNA, 1314 bp, Artificial Sequence
  synthetic OXOX variant sequence
aggcctaccgaaaatggtccccaaatcgtgatagcaaataacgctggtacctacttgcctgtgcctcgtggctccggtacaaaatcctccagcgccgctgacgccactcaaactgtgcctttcgctagtgatgacccaaatccccgcttgtggaatattgatacacaggacttgtctgttgtcgcccccgagcgcggtcagttgggtgctaagattttgggcccagataacttgcctattgacttgcaaaacgctgataccttggcaccacctaccactgattccgggtctattccaaatcccaagtggccttttgcccttagtcataacactttgtacactggaggctgggtccgtatacaaaatgacgaggttatgcccattgctaaggctatggcaggtgttaatatgcgcctggaagcaggtgcaatccgcgagctccattggcataatacagcagaatgggcctatatcttgaagggaactacccaaataacagccgtagatcaaaacggccgtaattatatggcaaacgtgggaccaggggatctctggtattttccccctgggatgccacattcactgcaaggtaccgacgcaaataacgagggaagcgaattcttgctgatatttccagacggaacttttgacgcgagcaaccagtttatgataactgattggttggctcacacccccaaagacgttattgccaagaatttcggtgtggacatttccgagttcgatcgtctgccatctcatgatctgtacatatatcctggggttgcccctccccttgacgctcaggcacccgaggaccctcagggtacaatacctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggaccttctttatcgagggacaggcacgtgtcactatttttgcaggccaaagtaatgcccaaactttcgactatcagggaggcgatatcgcttatatcccaaccgcctggggacactacgtagaaaattcagggaatacccagttgcgcttcctggagatattcaattcccctctcttcgaggatgtctcccttgcacaatggatcgctaataccccaccagccatcgtcaaggctacacttcaattgtccgacgaggttatcaacacattgaataagcagaaggctttcgttgtagcgtctgattga

SEQ ID NO: 63 - DNA, 1308 bp, Artificial Sequence
  synthetic OXOX variant sequence
aggcctaccgaaaatggtccccaaatcgtgatagcaaataacgctggtacctacttgcctgtgcctcgtggctccggtacaaaatcctccagcgccgctgacgccactcaaactgtgcctttcgctagtgatgacccaaatccccgcttgtgggatatggatacaaaaaatttggagaaagtcacaccccagcgcggtcagttgggtgctgagattttgggcccagataacttgcctattgacttgcaaaacgctgataccttggcaccacctaccactgattccgggtctattccaaatcccaagtggccttttgcccttagtcataacactttgtacactggaggctgggtccgtatacaaaatgacgaggttatgcccattgctaaggctatggcaggtgttaatatgcgcctggaagcaggtgcaatccgcgagatgcattggcataatacaccagaatgggcctacatcttgaagggaactacccaaataacagccgtagatcaaaatggccgtaattatttggcaaacgtgtctccaggggatctctggtattttcccgaagggatgccacattcactgcaaggtaccgacgcaaataacgagggaagcgaattcttgctgatatttccagacggaacttttgacgcgagcaaccagtttatgataactgattggttggctcacacccctaaagacgttattgccaagaatttcggtgtggacatttccgagttcgatcgtctgccatctcatgatctgtacatatttcctggggttgcccctccccttgacgctcgggcacccgaggaccctcagggtacagtccctctcccttacagttttgagttcagtaaggttaagcctacccaatatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcagggaggcgatatcgcttatatcccaaccgcctggggacactacgtagaaaattcagggaatacccagttgcgcttcctggagatattcaattcccctctctacgaggatgtctcccttgcacaatggatcgctaataccccaccagccatcgtcaaggctacacttcaattgtccgacgaggttatcaacacattgaataagcaaaaggctttcgttgtaggttga

SEQ ID NO: 64 - DNA, 1314 bp, Artificial Sequence
  synthetic OXOX variant sequence
aggcctaccgaaaatggtccccaaatcgtgatagcaaataacgctggtacctacttgcctgtgcctcgtggctccggtacaaaatcctccagcgccgctgacgccactcaaactgtgcctttcgctagtgatgacccaaatccccgcttgtggaatattgatacacaggacttgtctgttgtcgcccccgagcgcggtcagttgggtgctaagattttgggcccagataacttgcctattgacttgcaaaacgctgataccttggcaccacctaccactgattccgggtctattccaaaccccaagtggcctttctcccttagtcataacactttgtactcaggaggctacgtccgtatacaaaatgacgaggttatgcccattgctaaggctatggcaggtgttaatatgcgcctggaagcaggtgcaatccgtgagctccattggcataatacagcagaatgggcctatgttttgaagggaactacccaaataacagccgtagatcagaatggccgtaattatatggcaaacgtgggaccaggggatctctggtattttcccgaagggatgccacattcactgcaaggtaccgacgcaaataacgagggaagcgaattcttgctgatatttccagacggaacttttgactctagcaaccagtttatgataactgattggttggctcacacccctaaggacgttattgccaagaatttcggtgtggacatttccgagttcgatcgtctgccatctcatgatctgtacatatttcctggggttgcccctccccttgacgctaaagcacccgaggaccctcagggtacagtccctctccctttcagttttgagttcagtaaggttaagcctacccaatatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaactttcgactatcaggccggcgatatcgcttatatcccaaccgcctggggacactacgtagaaaattcagggaatacccagttgcgcttcctggagatattcaattccgatgtgtacgaggatgtctcccttgcacaatggatcgctaataccccaccagccatcgtcaaggctacacttcaattgtccgacgaggttatcaacacattgaataagcaaaaggctttcgttgtagcgtctgattga

SEQ ID NO: 65 - DNA, 1308 bp, Artificial Sequence
  synthetic OXOX variant sequence
aggcctaccgaaaatggtccccaaatcgtgatagcaaataacgctggtacctacttgcctgtgcttcgtggctccggtacaaaatcctccagcgccgctgaaccgactgaaactgtgcctttcgctagtgatgacccaaatccccgcttgtgggatattgatacacaggacttgtctgttgtcgcccccgagcgcggtcagttgggtgctaagattttgggcccagataacttgcctattgacttgcaaaacgctgataccttggcaccacctaccactgattccgggtctgttccaaatcccaagtggcctttcgcccttagtcataacactttgtactcaggaggctgggtccgtatacaaaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatcaaaatggccgtaattatttggcaaacgtgggaccaggggatctctggtattttcccgaagggatgccacattcactgcaaggtaccaacgcagcagacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatttcctggggttgcccctccccttgacgctactgcacccgaggaccctcagggtacagtccctctccctttcagttttgagttcagtaaggttgtgcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactctgtttgcaggccaaagtaatgcccaaacttacgactatcagggaggcgatatcgcttatatcccaaccgcctggggacactacgtagaaaattcagggaatacccagttgcgcttcctggagatattcaattcccctctcttcgaggatgtctcccttgcacaatggatcgctaataccccaccagccatcgtcaaggctacacttcaattgtccgacgaggttatcaacacattgaataagcagaaggcttccgttgtaggttga

SEQ ID NO: 66 - protein, 435 aa, Artificial Sequence
  synthetic OXOX variant sequence
RPTENGPQVVIANNAGTYLPVLRGSGTKSSSAADATQTVPFASDDPNPRLWDIDTKNLEKVTPERGQLGAKILGPDNLPIDLQNADTLAPPTTDSGSIPNPKWPFALSHNTLYSGGWVRIQNDEVMPIAKAMAGVNMRLEAGAIRELHWHNTPEWAYILKGTTQITAVDQNGRNYLANVGPGDLWYFPEGMPHSLQGTDANNEGSEFLLIFPDGTFDSSNQFMITDWLAHTPKDVIAKNFGVDISEFDRLPSHDLYIYPGVAPPLDAKAPEDPQGTVPLPYSFEFSKAKPTQYAGGTVKIADTRTFPIAKTISVAEVTVEPGAMRELHWHPTEDEWTYFIEGQARVTIFAGQSNAQTFDYQGGDIAYIPTAWGHYVENSGNTQLRFLEIFNSPLYEDVSLAQWIANTPPAIVKATLQLSDEVINTLNKQKAFVVG

SEQ ID NO: 67 - protein, 435 aa, Artficial Sequence
RPTENGPQIVIANNAGTYLPVLRGSGTKSSSAADATQTVPFASDDPNPRLWGIDTKNLEKVTPERGQLGAKILGPDNLPIDLQNADTLAPPTTDSGSVPNPKWPFALSHNTLYSGGWVRIQNDEVMPIAKAMAGVNMRLEAGAIRELHWHNTPEWAYILKGTTQITAVDQNGRNYLANVGPGDLWYFPEGMPHSLQGTDANNEGSEFLLIFPDGTFDSSNQFMITDWLAHTPKDVIAKNFGVDISEFDRLPSHDLYIYPGVAPPLDAKAPEDPQGTVPLPFSFEFSKVKPTQYAGGTVKIADTRTFPIAKTISVAEVTVEPGAMRELHWHPTEDEWTYFIEGQARVTIFAGQSNAQTFDYQGGDIAYIPTAWGHYVENSGNTQLRFLEIFNSPLYEDVSLAQWIANTPPAIVKATLQLSDEVINTLNKQKAFVVG

SEQ ID NO: 68 - protein, 435 aa, Artificial Sequence
  synthetic OXOX variant sequence
RPTENGPQTVIANNAGTYLSVLRGSGTKSSSAADATQTVPFASDDPNPRLWDIDTKNLEKVTPERGQLGAKILGPDNLPIDLQNADTLAPPTTDSGSVPNPKWPFALSHNTLYSGGWVRIQNDEVMPIAKAMAGVNMRLEAGAIRELHWHNTPEWAYILKGTTQITAVDQNGRNYLANVGPGDLWYFPEGMPHSLQGTDANNEGSEFLLIFPDGTFDSSNQFMITDWLAHTPKDVIAKNFGVDISEFDRLPSHDLYIYPGVAPPLDAKAPEDPQGTVPLPFSFEFSKVKPTQYAGGTVKIADTRTFPIAKTISVAEVTVEPGAMRELHWHPTEDEWTYFIEGQARVTIFAGQSNAQTFDYQGGDIAYIPTAWGHYVENSGNTQLRFLEIFNSPLYEDVSLAQWIANTPPAIVKATLQLSDEVINTLNKQKAFVVG

SEQ ID NO: 69 - protein, 435 aa, Artificial Sequence
  synthetic OXOX variant sequence
RPTENGPQIVIANNAGTYLPVLRGSGTKSSSAADATQTVPFASDDPNPRLWDIDTKNLEKVTPERGQLGAKILGPDNLPIDLQNADTLAPPTTDSGSVPNPKWPFALSHNTLYSGGWVRIQNDEVMPIAKAMAGVNMRLEAGAIRELHWHNTPEWAYILKGTTQITAVDQNGRNYLANVGPGDLWYFPEGMPHSLQGTDANNEGSEFLLIFPDGTFDSSNQFMITDWLAHTPKDVIAKNFGVDISEFDRLPSHDLYIYPGVAPPLDAKAPEDPQGTVPLPFSFEFSKVKPTQYAGGTVKIADTRTFPIAKTISVAEVTVEPGAMRELHWHPTEDEWTYFIEGQARVTIFAGQSNAQTFDYQGGDIAYIPTAWGHYVENSGNTQLRFLEIFNSPLYEDVSLAQWIANTPPAIVKATLQLSDEVINTLNKQKAFVVG

SEQ ID NO: 70 - protein, 435 aa, Artificial Sequence
  synthetic OXOX variant sequence
RPTENGPQIVIANNAGTYLPVLRGSGTKSSSAADATQTVPFASDDPNPRLWDIDTKNLEKVTPERGQLGAEILGPDNIPIDLQNADTLAPPTTDSGSIPNPKWPFALSHNTLYSGGYVRIQNDEVMPIAKAMAGVNMRLEAGAIRELHWHNTPEWAYILKGTTQITAVDQNGRNYLANVGPGDLWYFPEGIPHSLQGTDANNEGSEFLLIFPDGTFDSSNQFMITDWLAHTPKDVIAKNFGVDISEFDRLPSHDLYIFPGVAPPLDAKAPEDPQGTVPLPYSFEFSKVKPTQYAGGTVKIADTRTFPIAKTISVAEVTVEPGAMRELHWHPTEDEWTYFIEGQARVTIFAGQSNAQTYDYQGGDIAYIPTAWGHYVENSGNTQLRFLEIFNSPLYEDVSLAQWIANTPPAIVKATLQLSDEVINTLNKQKASVVG

SEQ ID NO: 71 - protein, 435 aa, Artificial Sequence
  synthetic OXOX variant sequence
RPTENGPQIVIANNAGTHLPVPRGSGTKSSSAADATQTVPFASDDPNPRLWDIDTQDLSVVAPERGPLGAKILGPDNLPIDLQNADTLAPPTTDSGSIPNPKWPFALSHNTLYSGGYVRIQNDEVMPIATAMAGVNMRLEAGAIRELHWHNTPEWAYILKGTTQITAVDQNGRNYMANVGPGDLWYFPPGMPHSLQGTDANNEGSEFLLIFPDGTFDASNQFMITDWLAHTPKDVIAKNFGVDTSEFDRLPSHDLYIFPGVAPPLDAQAPEDPQGTVPLPYSFEFSKVKPTQYAGGTVKIADTRTFPIAKTISVAEVTVEPGAMRELHWHPTEDEWTYFIEGQARVTIFAGQSNAQTFDYQGGDIAYIPTAWGHYVENSGNTQLRFLEIFNSDVYEDVSLAQWIANTPPAIVKATLQLSDEVINTLNKQKAFVVG

SEQ ID NO: 72 - protein, 435 aa, Artificial Sequence
  synthetic OXOX variant sequence
RPTENGPQIVIANNAGTHLPVPRGSGTKSSSAADATQTVPFASDDPNPRLWDIDTKNLEKVTPERGQLGAKILGPDNLPIDLQNADTLAPPTTDSGSVPNPKWPFALSHNTLYSGGYVRIQNDEVMPIAKAMAGVNMRLEAGAIRELHWHNTPEWAYILKGTTQITAVDQNGRNYMANVSPGDLWYFPPGMPHSLQGTDANNEGSEFLLIFPDGTFDSSNQFMITDWLAHTPKDVIAKNFGVDISEFDRLPSHDLYIFPGVAPPLDAKAPEDPQGTVPLPYSFEFSKVKPTQYAGGTVKIADTRTFPIAKTISVAEVTVEPGAMRELHWHPTEDEWTFFIEGQARVTIFAGQSNAQTFDYQGGDIAYIPTAYGHYVENSGNTQLRFLEIFNSDVYEDVSLAQWIANTPPAIVKATLQLSDEVIDSLNKQKASVVG

SEQ ID NO: 73 - protein, 437 aa, Artificial Sequence
  synthetic OXOX variant sequence
RPTENGPQIVIANNAGTYLPVPRGSGTKSSSAADATQTVPFASDDPNPRLWNIDTQDLSVVAPERGPLGAKILGPDNLPIDLQNADTLAPPTTDSGSVPNPKWPFALSHNTLYSGGYVRIQNDEVMPIAKAMAGVNMRLEAGAIREMHWHNTPEWAYILKGTTQITAVDQNGRNYLANVGPGDLWYFPPGMPHSLQGTDANNEGSEFLLIFPDGTFDSSNQFMITDWLAHTPKDVIAKNFGVDISEFDRLPSHDLYIFPGVAPPLDAKAPEDPQGTVPLPYSFEFSKVKPTQYAGGTVKIADTRTFPIAKTISVAEVTVEPGAMRELHWHPTEDEWTYFIEGQARVTIFAGQSNAQTFDYQGGDIAYIPTAWGHYVENSGNTQLRFLEIFNSPLYEDVSLAQWIANTPPAIVKATLQLSDEVINTLNKQKAFVVASD

SEQ ID NO: 74 - protein, 437 aa, Artificial Sequence
  synthetic OXOX variant sequence
RPTENGPQIVIANNAGTHLPVPRGSGTKSSSAADATQTVPFASDDPNPRLWDMDTKNLEKVTPERGQLGAKILGPDNLPIDLQNADTLAPPTTDSGSVPNPKWPFALSHNTLYSGGYVRIQNDEVLPIATAMAGVNMRLEAGAIRELHWHNTPEWAYILKGTTQITAVDQNGRNYMANVGPGDLWYFPPGMPHSLQGTDANNEGSEFLLIFPDGTFDSSNQFMITDWLAHTPKDVIAKNFGVDISEFDRLPSHDLYIFPGVAPPLDAKAPEDPQGTVPLPYSFEFSKVKPTQYAGGTVKIADTRTFPIAKTISVAEVTVEPGAMRELHWHPTEDEWTFFIEGQARVTIFAGQSNAQTFDYQAGDIAYIPTAWGHYVENSGNTQLRFLEIFNSPLYEDVSLAQWIANTPPAIVKATLQLSDEVINTLNKQKAFVVASD

SEQ ID NO: 75 - protein, 435 aa, Artificial Sequence
  synthetic OXOX variant sequence
RPTENGPQIVIANNAGTYLPVLRGSGTKSSSAADATQTVPFASDDPNPRLWDMDTKNLEKVTPERGPLGAKILGPDNLPIDLQNADTLAPPTTDSGSVPNPKWPFALSHNTLYSGGYVRIQNDEVMPIAKAMAGVNMRLEAGAIREMHWHNTPEWAYVLKGTTQITAVDQNGRNYLANVGPGDLWYFPEGVPHSLQGTDANNEGSEFLLIFPDGTFDSSNQFMITDWLAHTPKDVIAKNFGVDISEFDRLPSHDLYIFPGVAPPLDAKAPEDPQGTVPLPYSFEFSKVKPTQYAGGTVKIADTRTFPIAKTISVAEVTVEPGAMRELHWHPTEDEWTYFIEGQARVTIFAGQSNAQTFDYQAGDIAYIPTAWGHYVENSGNTQLRFLEIFNSPLFEDVSLAQWIANTPPAIVKATLQLSDEVINTLNKQKASVVG

SEQ ID NO: 76 - protein, 435 aa, Artificial Sequence
  synthetic OXOX variant sequence
RPTENGPQIVIANNAGTHLPVPRGSGTKSSSAADATQTVPFASDDPNPRLWDMDTKNLEKVTPERGQLGAKILGPDNLPIDLQNADTLAPPTTDSGSVPNPKWPFALSHNTLYSGGWVRIQNDEVMPIAKAMAGVNMRLEAGAIREMHWHNTPEWAYVLKGTTQITAVDENGRNYMANVGPGDLWYFPPGMPHSLQGTDANNEGSEFLLIFPDGTFDSSNQFMITDWLAHTPKDVIAKNFGVDISEFDRLPSHDLYIFPGVAPPLDAKAPEDPQGTVPLPFSFEFSKVKPTQYAGGTVKIADTRTFPIAKTISVAEVTVEPGAMRELHWHPTEDEWTFFIEGQARVTIFAGQSNAQTYDYQGGDIAYIPTAWGHYVENSGNTQLRFLEIFNSDVYEDVSLAQWIANTPPAIVKATLQLSDEVINTLNKQKASVVG

SEQ ID NO: 77 - protein, 437 aa, Artificial Sequence
  synthetic OXOX variant sequence
RPTENGPQIVIANNAGTYLPVLRGSGTKSSSAADATQTVPFASDDPNPRLWDMDTKNLEKVTPERGQLGAKILGPDNLPIDLQNADTLAPPTTDSGSIPNPKWPFALSHNTLYSGGYVRIQNDEVMPIAKAMAGVNMRLEAGAIRELHWHNTPEWAYILKGTTQITAVDQNGRNYMANVSPGDLWYFPEGMPHSLQGTDANNEGSEFLLIFPDGTFDSSNQFMITDWLAHTPKDVIAKNFGVDISEFDRLPSHDLYIFPGVAPPLDAKAPEDPQGTVPLPFSFEFSKVKPTQYAGGTVKIADTRTFPIAKTISVAEVTVEPGAMRELHWHPTEDEWTYFIEGQARVTIFAGQSNAQTFDYQAGDIAYIPTAWGHYVENSGNTQLRFLEIFNSPLYEDVSLAQWIANTPPAIVKATLQLSDEVIDSLNKQKAFVVASD

SEQ ID NO: 78 - protein, 435 aa, Artificial Sequence
  synthetic OXOX variant sequence
RPTENGPQIVIANNAGTYLPVLRGSGTKSSSAADATQTVPFASDDPNPRLWDIDTQDLSVVAPERGPLGAKILGPDNLPIDLQNADTLAPPTTDSGSVPNPKWPFALSHNTLYSGGYVRIQNNEVLPIAKAMAGVNMRLEAGAIRELHWHNTPEWAYVLKGTTQITAVDENGRNYMANVGPGDLWYFPEGIPHSLQGTDANNEGSEFLLIFPDGTFDSSNQFMITDWLAHTPKDVIAKNFGVDISEFDRLPSHDLYIFPGVAPPLDAQAPEDPQGTVPLPYSFEFSKVKPTQYAGGTVKIADTRTFPIAKTISVAEVTVEPGAMRELHWHPTEDEWTYFIEGQARVTIFAGQSNAQTFDYQGGDIAYIPTAWGHYVENSGNTQLRFLEIFNSPLFEDVSLAQWIANTPPAIVKATLQLSDEVIDSLNKQKASVVG

SEQ ID NO: 79 - protein, 437 aa, Artificial Sequence
  synthetic OXOX variant sequence
RPTENGPQIVIANNAGTYLPVPRGSGTKSSSAADATQTVPFASDDPNPRLWNIDTKNLIKVTPERGQLGAKILGPDNLPIDLQNADTLAPPTTDSGSIPNPKWPFALSHNTLYTGGWVRIQNDEVLPIAKAMAGVNMRLEAGAIREMHWHNTPEWAYILKGTTQITAVDENGRNYMANVGPGDLWYFPPGMPHSLQGTDANNEGSEFLLIFPDGTFDSSNQFMITDWLAHTPKDVIAKNFGVDISEFDRLPSHDLYIYPGVAPPLDAQAPEDPQGTVPLPYSFEFSKVKPTQYAGGTVKIADTRTFPIAKTISVAEVTVEPGAMRELHWHPTEDEWTYFIEGQARVTIFAGQSNAQTFDYQGGDIAYIPTAWGHYVENSGNTQLRFLEIFNSPLFEDVSLAQWIANTPPAIVKATLQLSDEVINTFNKQKAFVVASD

SEQ ID NO: 80 - protein, 435 aa, Artificial Sequence
  synthetic OXOX variant sequence
RPTENGPQIVIANNAGTYLPVPRGSGTKSSSAADATQTVPFASDDPNPRLWDMDTKNLEKVTPQRGQLGAEILGPDNLPIDLQNADTLAPPTTDSGSVPNPKWPFALSHNTLYSGGWVRIQNDEVMPIATAMAGVNMRLEAGAIRELHWHNTPEWAYILKGTTQITAVDENGKNYLANVGPGDLWYFPEGVPHSLQGTDANNEGSEFLLIFPDGTFDSSNQFMITDWLAHTPKDVIAKNFGVDISEFDRLPSHDLYIFPGVAPPLDAQAPEDPQGTVPLPYSFEFSKVKPTQYAGGTVKIADTRTFPIAKTISVAEVTVEPGAMRELHWHPTEDEWTYFIEGQARVTIFAGQSNAQTFDYQGGDIAYIPTAWGHYVENSGNTQLRFLEIFNSDVYEDVSLAQWIANTPPAIVKATLQLSDEVINTLNKQKAFVVG

SEQ ID NO: 81 - protein, 435 aa, Artificial Sequence
  synthetic OXOX variant sequence
RPTENGPQIVIANNAGTYLPVLRGSGTKSSSAADATQTVPFASDDPNPRLWNIDTQDLSVVAPERGPLGAKILGPDNLPIDLQNADTLAPPTTDSGSVPNPKWPFALSHNTLYSGGYVRIQNDEVMPIAKAMAGVNMRLEAGAIREMHWHNTPEWAYILKGTTQITAVDQNGRNYMANVGPGDLWYFPEGMPHSLQGTDANNEGSEFLLIFPDGTFDSSNQFMITDWLAHTPKDVIAKNFGVDISEFDRLPSHDLYIFPGVAPPLDAKAPEDPQGTVPLPYSFEFSKVKPTQYAGGTVKIADTRTFPIAKTISVAEVTVEPGAMRELHWHPTEDEWTFFIEGQARVTIFAGQSNAQTFDYQAGDIAYIPTAWGHYVENSGNTQLRFLEIFNSPLFEDVSLAQWIANTPPAIVKATLQLSDEVINTLNKQKAFVVG

SEQ ID NO: 82 - protein, 435 aa, Artificial Sequence
  synthetic OXOX variant sequence
RPTENGPQIVIANNAGTHLPVPRGSGTKSSSAADATQTVPFASDDPNPRLWDMDTKNLEKVTPERGQLGAEILGPDNLPIDLQNADTLAPPTTDSGSIPNPKWPFALSHNTLYSGGYVRIQNDEVMPIAKAMAGVNMRLEAGAIREMHWHNTQEWAYILKGTTQITAVDQNGRNYLANVGPGDLWYFPEGMPHSLQGTDANNEGSEFLLIFPDGTFDSSNQFMITDWLAHTPKDVIAKNFGVDISEFDRLPSHDLYIFPGVAPPLDAKAPEDPQGTVPLPYSFEFSKVKPTQYAGGTVKIADTRTFPIAKTISVAEVTVEPGAMRELHWHPTEDEWTYFIEGQARVTIFAGQSNAQTFDYQAGDIAYIPTAWGHYVENSGNTQLRFLEIFNSPLYEDVSLAQWIANTPPAIVKATLQLSDEVINTLNKQKAFVVG

SEQ ID NO: 83 - protein, 435 aa, Artificial Sequence
  synthetic OXOX variant sequence
RPTENGPQIVIANNAGTHLPVPRGSGTKSSSAADATQTVPFASDDPNPRLWDMDTKNLEKVTPQRGQLGAEILGPDNLPIDLQNADTLAPPTTDSGSIPNPKWPFALSHNTLYSGGYVRIQNDEVMPIAKAMAGVNMRLEAGAIRELHWHNTPEWAYILKGTTQITAVDQNGRNYLANVSPGDLWYFPEGMPHSLQGTDANNEGSEFLLIFPDGTFDSSNQFMITDWLAHTPKDVIAKNFGVDISEFDRLPSHDLYIFPGVAPPLDAKAPEDPQGTVPLPYSFEFSKVKPTQYAGGTVKIADTRTFPIAKTISVAEVTVEPGAMRELHWHPTEDEWTYFIEGQARVTIFAGQSNAQTFDYQAGDIAYIPTAWGHYVENSGNTQLRFLEIFNSPLYEDVSLAQWIANTPPAIVKATLQLSDEVINTLNKQKAFVVG

SEQ ID NO: 84 - protein, 437 aa, Artificial Sequence
  synthetic OXOX variant sequence
RPTENGPQIVIANNAGTYLPVPRGSGTKSSSAADATQTAPFASDDPNPRLWDMDTKNLEKVQPERGQLGAKILGPDNLPIDLQNADTLAPPTTDSGSIPNPKWPFALSHNTLYSGGYVRIQNDEVMPIAKAMAGVNMRLEAGAIRELHWHNTPEWAYILKGTTQITAVDQNGRNYMANVGPGDLWYFPPGMPHSLQGTDANNEGSEFLLIFPDGTFDSSNQFMITDWLAHTPKDVIAKNFGVDISEFDRLPSHDLYIFPGVAPPLDAQAPEDPQGTVPLPYSFEFSKVKPTQYAGGTVKIADTRTFPIAKTISVAEVTVEPGAMRELHWHPTEDEWTYFIEGQARVTIFAGQSNAQTYDYQGGDIAYIPTAWGHYVENSGNTQLRFLEIFNSPLFEDVSLAQWIANTPPAIVKATLQLSDEVINTFNKQKAFVVASD

SEQ ID NO: 85 - protein, 435 aa, Artificial Sequence
  synthetic OXOX variant sequence
RPTENGPQIVIANNAGTYLPVLRGSGTKSSSAADATQAVPFASDDPNPRLWDMDTKNLEKVTPERGQLGAEILGPDNLPIDLQNADTLAPPTTDSGSVPNPKWPFALSHNTLYSGGWVRIQNNEVLPIAKAMAGVNMRLEAGAIREMHWHNTPEWAYILKGTTQITAVDQNGKNYLANVGPGDLWYFPPGMPHSLQGTDANNEGSEFLLIFPDGTFDSSNQFMITDWLAHTPKDVIAKNFGVDISEFDRLPSHDLYIFPGVAPPLDAEAPEDPQGTVPLPYSFEFSKVKPTQYAGGTVKIADTRTFPIAKTISVAEVTVEPGAMRELHWHPTEDEWTFFIEGQARVTIFAGQSNAQTFDYQGGDIAYIPTAWGHYVENSGNTQLRFLEIFNSPLFEDVSLAQWIANTPPAIVKATLQLSDEVIDSLNKQKAFVVG

SEQ ID NO: 86 - protein, 435 aa, Artificial Sequence
  synthetic OXOX variant sequence
RPTENGPQIVIANNAGTYLPVPRGSGTKSSSAADATQTVPFASDDPNPRLWDMDTKNLEKVTPERGQLGAKILGPDNLPIDLQNADTLAPPTTDSGSIPNPKWPFALSHNTLYSGGYVRIQNDEVMPIAKAMAGVNMRLEAGAIREMHWHNTPEWAYILKGTTQITAVDQNGRNYMANVGPGDLWYFPEGMPHSLQGTDANNEGSEFLLIFPDGTFDSSNQFMITDWLAHTPKDVIAKNFGVDISEFDRLPSHDLYIYPGVAPPLDAKAPEDPQGTVPLPYSFEFSKVKPTQYAGGTVKIADTRTFPIAKTISVAEVTVEPGAMRELHWHPTEDEWTYFIEGQARVTIFAGQSNAQTYDYQGGDIAYIPTAWGHYVENSGNTQLRFLEIFNSPLFEDVSLAQWIANTPPAIVKATLQLSDEVINTLNKQKAFVVG

SEQ ID NO: 87 - protein, 435 aa, Artificial Sequence
  synthetic OXOX variant sequence
RPTENGPQIVIANNAGTHLPVPRGSGTKSSSAADATQTVPFASDDPNPRLWDMDTKNLEKVTPERGQLGAKILGPDNLPIDLQNADTLAPPTTDSGSIPNPKWPFALSHNTLYSGGYVRIQNDEVMPIATAMAGVNMRLEAGAIRELHWHNTPEWAYVLKGTTQITAVDQNGRNYMANVGPGDLWYFPEGVPHSLQGTDANNEGSEFLLIFPDGTFDSSNQFMITDWLAHTPKDVIAKNFGVDISEFDRLPSHDLYIYPGVAPPLDAKAPEDPQGTVPLPYSFEFCKVKPTQYAGGTVKIADTRTFPIAKTISVAEVTVEPGAMRELHWHPTEDEWTFFIEGQARVTIFAGQSNAQTFDYQGGDIAYIPTAWGHYVENSGNTQLRFLEIFNSPLFEDVSLAQWIANTPPAIVKATLQLSDEVINTLNKQKAFVVG

SEQ ID NO: 88 - protein, 435 aa, Artificial Sequence
  synthetic OXOX variant sequence
RPTENGPQIVIANNAGTHLPVPRGTGTKSSSAADATQTVPFASDDPNPRLWDMDTKNLEKVTPERGQLGAKILGPDNLPIDLQNADTLAPPTTDSGSIPNPKWPFALSHNTLYSGGWVRIQNNEVLPIATAMAGVNMRLEAGAIRELHWHNTPEWAYVLKGTTQITAVDQNGRNYMANVGPGDLWYFPPGMPHSLQGTDANNEGSEFLLIFPDGTFDASNQFMITDWLAHTPKDVIAKNFGVDISEFDRLPSHDLYIFPGVAPPLDAQAPEDPQGTVPLPYSFEFSKVKPTQYAGGTVKIADTRTFPIAKTISVAEVTVEPGAMRELHWHPTEDEWTYFIEGQARVTIFAGQSNAQTFDYQGGDIAYIPTAWGHYVENSGNTQLRYLEIFNSPLFEDISLAQWIANTPPAIVKATLQLSDEVINTLNKQKASVVG

SEQ ID NO: 89 - protein, 437 aa, Artificial Sequence
  synthetic OXOX variant sequence
RPTENGPQIVIANNAGTYLPVPRGSGTKSSSAADATQTVPFASDDPNPRLWNIDTQDLSVVAPERGQLGAKILGPDNLPIDLQNADTLAPPTTDSGSIPNPKWPFALSHNTLYTGGWVRIQNDEVMPIAKAMAGVNMRLEAGAIRELHWHNTAEWAYILKGTTQITAVDQNGRNYMANVGPGDLWYFPPGMPHSLQGTDANNEGSEFLLIFPDGTFDASNQFMITDWLAHTPKDVIAKNFGVDISEFDRLPSHDLYIYPGVAPPLDAQAPEDPQGTIPLPYSFEFSKVKPTQYAGGTVKIADTRTFPIAKTISVAEVTVEPGAMRELHWHPTEDEWTFFIEGQARVTIFAGQSNAQTFDYQGGDIAYIPTAWGHYVENSGNTQLRFLEIFNSPLFEDVSLAQWIANTPPAIVKATLQLSDEVINTLNKQKAFVVASD

SEQ ID NO: 90 - protein, 435 aa, Artificial Sequence
  synthetic OXOX variant sequence
RPTENGPQIVIANNAGTYLPVPRGSGTKSSSAADATQTVPFASDDPNPRLWDMDTKNLEKVTPQRGQLGAEILGPDNLPIDLQNADTLAPPTTDSGSIPNPKWPFALSHNTLYTGGWVRIQNDEVMPIAKAMAGVNMRLEAGAIREMHWHNTPEWAYILKGTTQITAVDQNGRNYLANVSPGDLWYFPEGMPHSLQGTDANNEGSEFLLIFPDGTFDASNQFMITDWLAHTPKDVIAKNFGVDISEFDRLPSHDLYIFPGVAPPLDARAPEDPQGTVPLPYSFEFSKVKPTQYAGGTVKIADTRTFPIAKTISVAEVTVEPGAMRELHWHPTEDEWTYFIEGQARVTIFAGQSNAQTFDYQGGDIAYIPTAWGHYVENSGNTQLRFLEIFNSPLYEDVSLAQWIANTPPAIVKATLQLSDEVINTLNKQKAFVVG

SEQ ID NO: 91 - protein, 437 aa, Artificial Sequence
  synthetic OXOX variant sequence
RPTENGPQIVIANNAGTYLPVPRGSGTKSSSAADATQTVPFASDDPNPRLWNIDTQDLSVVAPERGQLGAKILGPDNLPIDLQNADTLAPPTTDSGSIPNPKWPFSLSHNTLYSGGYVRIQNDEVMPIAKAMAGVNMRLEAGAIRELHWHNTAEWAYVLKGTTQITAVDQNGRNYMANVGPGDLWYFPEGMPHSLQGTDANNEGSEFLLIFPDGTFDSSNQFMITDWLAHTPKDVIAKNFGVDISEFDRLPSHDLYIFPGVAPPLDAKAPEDPQGTVPLPFSFEFSKVKPTQYAGGTVKIADTRTFPIAKTISVAEVTVEPGAMRELHWHPTEDEWTYFIEGQARVTIFAGQSNAQTFDYQAGDIAYIPTAWGHYVENSGNTQLRFLEIFNSDVYEDVSLAQWIANTPPAIVKATLQLSDEVINTLNKQKAFVVASD

SEQ ID NO: 92 - protein, 435 aa, Artificial Sequence
  synthetic OXOX variant sequence
RPTENGPQIVIANNAGTYLPVLRGSGTKSSSAAEPTETVPFASDDPNPRLWDIDTQDLSVVAPERGQLGAKILGPDNLPIDLQNADTLAPPTTDSGSVPNPKWPFALSHNTLYSGGWVRIQNDEVMPIAKAMAGVNMRLEAGAIRELHWHNTPEWAYILKGTTQITAVDQNGRNYLANVGPGDLWYFPEGMPHSLQGTNAADEGSEFLLIFPDGTFDSSNQFMITDWLAHTPKDVIAKNFGVDISEFDRLPSHDLYIFPGVAPPLDATAPEDPQGTVPLPFSFEFSKVVPTQYAGGTVKIADTRTFPIAKTISVAEVTVEPGAMRELHWHPTEDEWTYFIEGQARVTLFAGQSNAQTYDYQGGDIAYIPTAWGHYVENSGNTQLRFLEIFNSPLFEDVSLAQWIANTPPAIVKATLQLSDEVINTLNKQKASVVG

SEQ ID NO: 93 - DNA, 1308 bp, Artificial Sequence
  synthetic OXOX variant sequence
aggcctaccgaaaatggtccccaaatcgtgatagcaaataacgctggtacctacttgcctgtgcttcgtggctccggtacaaaatcctccagcgccgctgaaccgactgaaactgtgcctttcgctagtgatgacccaaatccccgcttgtgggatattgatacacaggacttgtctgttgtcgcccccgagcgcggtcagttgggtgctaagattttgggcccagataacttgcctattgacttgcaaaacgctgataccttggcaccacctaccactgattccgggtctgttccaaatcccaagtggcctttcgcccttagtcataacactttgtactcaggaggctgggtccgtatacaaaatgacgaggttatgcccattgctaaggctatggcaggtgttaatatgcgcctggaagcaggtgcaatccgcgagctccattggcataatacaccagaatgggcctatatcttgaagggaactgatcaaataacagccgtagatcaaaatggccgtaattatttggcaaacgtgggaccaggggatctctggtattttcccgaagggatgccacattcactgcaaggtaccaacgcagcagacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatttcctggggttgcccctccccttgacgctactgcacccgaggaccctcagggtacagtccctctccctttcagttttgagttcagtaaggttgtgcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactctgtttgcaggccaaagtaatgcccaaacttacgactatcagggaggcgatatcgcttatatcccaaccgcctggggacactacgtagaaaattcagggaatacccagttgcgcttcctggagatattcaattcccctctcttcgaggatgtctcccttgcacaatggatcgctaataccccaccagccatcgtcaaggctacacttcaattgtccgacgaggttatcaacacattgaataagcagaaggcttccgttgtaggttga

SEQ ID NO: 94 - DNA, 1314 bp, Artificial Sequence
  synthetic OXOX variant sequence
aggcctaccgaaaatggtccccaaatcgtgatagcaaataacgctggtacctacttgcctgtgcctcgtggctccggtacaaaatcctccagcgccgctgacgccactcaaactgtgcctttcgctagtgatgacccaaatccccgcttgtggaatattgatacacaggacttgtctgttgtcgcccccgagcgcggtcagttgggtgctaagattttgggcccagataacttgcctattgacttgcaaaacgctgataccttggcaccacctaccactgattccgggtctattccaaatcccaagtggcctttcgcccttagtcataacactttgtactcaggaggctacgtccgtatacaaaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatcaaaacggccgtaattatatggcaaacgtgggaccaggggatctctggtattttccccctgggatgccacattcactgcaaggtaccgacgcaaataacgagggatctgaattcttgctgatatttccagacggaacttttgactctagcaaccagtttatgataactgattggttggctcacacccccaaagacgttattgccaagaatttcggtgtggacatttccgagttcgatcgtctgccatctcatgatctgtacatatttcctggggttgcccctccccttgacgctaaagcgcccgaggaccctcagggtacagtccctctccctttcagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactctgtttgcaggccaaagtaatgcccaaactttcgactatcagggaggcgatatcgcttatatcccaaccgcctggggacactacgtagaaaattcagggaatacccagttgcgcttcctggagatattcaattcccctctcttcgaggatgtctcccttgcacaatggatcgctaataccccaccagccatcgtcaaggctacacttcaattgtccgacgaggttatcaacacattgaataagcagaaggctttcgttgtagcgtctgattga

SEQ ID NO: 95 - DNA, 1314 bp, Artificial Sequence
  synthetic OXOX variant sequence
aggcctaccgaaaatggtccccaaatcgtgatagcaaataacgctggtacctacttgcctgtgcctcgtggctccggtacaaaatcctccagcgccgctgacgccactcaaactgtgcctttcgctagtgatgacccaaatccccgcttgtgggatattgatacacaggacttgtctgttgtcgcccccgagcgcggtcagttgggtgctaagattttgggcccagataacttgcctattgacttgcaaaacgctgataccttggcaccacctaccactgattccgggtctgttccaaatcccaagtggcctttcgcccttagtcataacactttgtactcaggaggctgggtccgtatacaaaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatcaaaatggccgtaattatatggcaaacgtgggaccaggggatctctggtattttccccctgggatgccacattcactgcaaggtaccgacgcaaataacgagggaagcgaattcttgctgatatttccagacggaacttttgacgcgagcaaccagtttatgataactgattggttggctcacacccctaaagacgttattgccaagaatttcggtgtggacatttccgagttcgatcgtctgccgtctcatgatctgtacatatttcctggggttgcccctccccttgacgctactgcacccgaggaccctcagggtacagtccctctccctttcagttttgagttcagtaaggttaagcctacccaatatgccggtggtaccgttaaaatagctgatactcgtaccttccccatcgctaagaccatttctgttgctgaggttaccgtagaacctggagctatgcgcgagcttcactggcatcctactgaggatgagtggacctactttatcgagggacaggcacgtgtcactctgtttgcaggccaaagtaatgcccaaactttcgactatcagggaggcgatatcgcttatatcccaaccgcctggggacactacgtagaaaattcagggaatacccagttgcgcttcctggagatattcaattcccctctcttcgaggatgtctcccttgcacaatggatcgctaataccccaccagccatcgtcaaggctacacttcaattgtccgacgaggttatcaacacactgaataagcagaaggcttccgttgtagcgtctgattga

SEQ ID NO: 96 - DNA, 1314 bp, Artificial Sequence
  synthetic OXOX variant sequence
aggcctaccgaaaatggtccccaaatcgtgatagcaaataacgctggtacctacttgcctgtgcctcgtggctccggtacaaaatcctccagcgccgctgacgccactcaaactgcgcctttcgctagtgatgacccaaatccccgcttgtggaatattgatacacaggacttgtctgttgtcgcccccgagcgcggtcagttgggtgctaagattttgggcccagataacttgcctattgacttgcaaaacgctgataccttggcaccacctaccactgattccgggtctgttccaaatcccaagtggccttttgcccttagtcataacactttgtactcaggaggctgggtccgtatacaaaatgacgaggttatgcccattgctaaggctatggcaggtgttaatatgcgcctggaagcaggtgcaatccgcgagctccattggcataatacaccagaatgggcctatatcttgaagggaactacccaaataacagccgtagatcaaaacggccgtaattatatggcaaacgtgggaccaggggatctctggtattttcccgaagggatgccacattcactgcaaggtaccgacgcaaataacgagggatctgaattcttgctgatatttccagacggaacttttgacgcgagcaaccagtttatgataactgattggttggctcacacccctaaagacgttattgccaagaatttcggtgtggacatttccgagttcgatcgtctgccatctcatgatctgtacatatttcctggggttgcccctccccttgacgctcaggcacccgaggaccctcagggtacagtccctctcccttacagttttgagttcagtaaggttaagcctacccagtatgccggtggtactgttaaaatagctgatactcgtaccttccccatcgccaagaccatttctgttgctgaggttaccgtagaacctggagctatgcgcgagcttcactggcatcctactgaggatgagtggacctactttatcgagggacaggcacgtgtcactatttttgcaggccaaagtaatgcccaaacttacgactatcagggaggcgatatcgcttatatcccaaccgcctggggacactacgtagaaaattcagggaatacccagttgcgcttcctggagatattcaattcccctctctacgaggatgtctcccttgcacaatggatcgctaataccccaccagccatcgtcaaggctacacttcaattgtccgacgaggttatcaacacactgaataagcagaaggctttcgttgtagcgtctgattga

SEQ ID NO: 97 - DNA, 1308 bp, Artificial Sequence
  synthetic OXOX variant sequence
aggcctaccgaaaatggtccccaaatcgtgatagcaaataacgctggtacctacttgcctgtgcttcgtggctccggtacaaaatcctccagcgccgctgacgccactcaaactgtgcctttcgctagtgatgacccaaatccccgcttgtgggatattgatacaaaaaatttggagaaagccacacccgagcgcggtcagttgggtgctgagattttgggcccagataacttgcctctggacatccaaaacgctgataccttggcaccacctaccactgattccgggtctattccaaatcccaagtggcctttcgcccttagtcataacactttgtactcaggaggctgggtccgtatacaaaatgacgaggttatgcccattgctaaggctatggcaggtgttaatatgcgcctggaagcaggtaccatccgcgagctccattggcataatacaccagaatgggcctatatcttgaagggaactacccaaataacagccgtagatcaaaatggccgtaattatttggcaaacgtgggaccaggggatctctggtattttcccgaagggatcccacattcactgcaaggtaccgacgcaaataacgagggaagcgaattcttgctgatatttccagacggaacttttgacgcgagcaaccagtttatgataactgattggttggctcacacccctaaagacgttattgccaagaatttcggtgtggacatttccgagttcgatcgtctgccatctcatgatctgtacatatttcctggggttgcccctccccttgacgctactgcacccgaggaccctcagggtacaatacctctcccttacagttttgagttcagtaaggttaagcctacccagtatgccggtggtactgttaaaatagctgatactcgtaccttccccatcgctaagaccatttctgttgctgaggttaccgtagaacctggagctatgcgcgagcttcactggcatcctactgaggatgagtggaccttctttatcgagggacaggcacgtgtcactctgtttgcaggccaaagtaatgcccaaacttacgactatcagggaggcgatatcgcttatatcccaaccgcctggggacactacgtagaaaattcagggaatacccagttgcgcttcctggagatattcaattcccctctcttcgaggatgtctcccttgcacaatggatcgctaataccccaccagccatcgtcaaggctacacttcaattgtccgacgaggttatcaacacattgaataagcagaaggcttccgttgtaggttga

SEQ ID NO: 98 - DNA, 1308 bp, Artificial Sequence
  synthetic OXOX variant sequence
aggcctaccgaaaatggtccccaaatcgtgatagcaaataacgctggtacctacttgcctgtgcttcgtggctccggtacaaaatcctccagcgccgctgacgccactcaaactgtgcctttcgctagtgatgacccaaatccccgcttgtgggatatggatacaaaaaatttggagaaagtcacacccgagcgcggtcagttgggtgctaagattttgggcccagataacttgcctattgacttgcaaaacgctgataccttggcaccacctaccactgattccgggtctattccaaatgcaaagtggcctttcgcccttagtcataacactttgtactcaggaggctgggtccgtatacaaaatgacgaggttatgcccattgctaaggctatggcaggtgttaatatgcgcctggaagcaggtaccatccgcgagctccattggcataatacaccagaatgggcctatatcttgaagggaactacccaaataacagccgtagatcaaaatggccgtaattatttggcaaacgtgggaccaggggatctctggtattttcccgaagggatcccacattcactgcaaggtaccaacgcagcagacgagggaagcgaattcttgctgatatttccagacggaacttttgactctagcaaccagtttatgataactgattggttggctcacacccctaaagacgttattgccaagaatttcggtgtggacatttccgagttcgatcgtctgccatctcatgatctgtacatatttcctggggttgcccctccccttgacgctaaagcacccgaggaccctcagggtacaatacctctcccttacagttttgagttcagtaaggttaagcctacccagtatgccggtggtactgttaaaatagctgatactcgtactttccccatcgctaagaccatttctgttgctgaggttaccgtagaacctggagctatgcgcgagcttcactggcatcctactgaggatgagtggacctactttatcgagggacaggcacgtgtcactctgtttgcaggcgaaagtaatgcccaaacttacgactatcagggaggcgatatcgcttatatcccaaccgcctggggacactacgtagaaaattcagggaatacccagttgcgcttcctggagatattcaattcccctctcttcgaggatgtctcccttgcacaatggatcgctaacaccccaccagccatcgtcaaggctacacttcaattgtccgacgaggttatcaacacattgaataagcagaaggcttccgttgtaggttga

SEQ ID NO: 99 - DNA, 1308 bp, Artificial Sequence
  synthetic OXOX variant sequence
aggcctaccgaaaatggtccccaaatcgtgatagcaaataacgctggtacctacttgcctgtgcttcgtggctccggtacagaatcctccagcgccgctgaaccgactgaaactgtgcctttcgctagtgatgacccaaatccccgcttgtgggatattgatacaaaaaatttggagaaagtcacacccgagcgcggtcagttgggtgctaagattttgggcccagataacttgcctattgacttgcaaaacgctgataccttggcaccacctaccactgattccgggtctattccaaatcccaagtggcctttcgcccttagtcataacactttgtactcaggaggctgggtccgtatacaaaatgacgaggttatgcccattgctaaggctatggcaggtgttaatatgcgcctggaagcaggtaccatccgcgagctccattggcataatacaccagaatgggcctatatcttgaagggaactacccaaataacagccgtagatcaaaatggccgtaattatttggcaaacgtgggaccaggggatctctggtattttcccgaggggatcccacattcactgcaaggtaccgacgcaaataacgagggaagcgaattcttgctgatatttccagacggaacttttgacgcgagcaaccagtttatgataactgattggttggctcacacccctaaagacgttattgccaagaatttcggtgtggacatttccgagttcgatcgtctgccatctcatgatctgtacatatttcctggggttgcccctccccttgacgctactgcacccgaggaccctcagggtacagtccctctcccttacagttttgagttcagtaaggttgtgcctacccagtatgccggtggtactgttaaaatagctgatactcgtaccttccccatcgctaagaccatttctgttgctgaggttaccgtagaacctggagctatgcgcgagcttcactggcatcctactgaggatgagtggaccttctttatcgagggacaggcacgtgtcactatttttgcaggcgaaagtaatgcccaaacttacgactatcagggaggcgatatcgcttatatcccaaccgcctggggacactacgtagaaaattcagggaatacccagttgcgcttcctggagatattcaattcccctctctacgaggatgtctcccttgcacaatggatcgctaataccccacgtgccatcgtcaaggctacacttcaattgtccgacgaggttatcaacacattgaataagcagaaggcttccgttgtaggttga

SEQ ID NO: 100 - protein, 435 aa, Artificial Sequence
  synthetic OXOX variant sequence
RPTENGPQIVIANNAGTYLPVLRGSGTKSSSAAEPTETVPFASDDPNPRLWDIDTQDLSVVAPERGQLGAKILGPDNLPIDLQNADTLAPPTTDSGSVPNPKWPFALSHNTLYSGGWVRIQNDEVMPIAKAMAGVNMRLEAGAIRELHWHNTPEWAYILKGTDQITAVDQNGRNYLANVGPGDLWYFPEGMPHSLQGTNAADEGSEFLLIFPDGTFDSSNQFMITDWLAHTPKDVIAKNFGVDISEFDRLPSHDLYIFPGVAPPLDATAPEDPQGTVPLPFSFEFSKVVPTQYAGGTVKIADTRTFPIAKTISVAEVTVEPGAMRELHWHPTEDEWTYFIEGQARVTLFAGQSNAQTYDYQGGDIAYIPTAWGHYVENSGNTQLRFLEIFNSPLFEDVSLAQWIANTPPAIVKATLQLSDEVINTLNKQKASVVG

SEQ ID NO: 101 - protein, 437 aa, Artificial Sequence
  synthetic OXOX variant sequence
RPTENGPQIVIANNAGTYLPVPRGSGTKSSSAADATQTVPFASDDPNPRLWNIDTQDLSVVAPERGQLGAKILGPDNLPIDLQNADTLAPPTTDSGSIPNPKWPFALSHNTLYSGGYVRIQNDEVMPIAKAMAGVNMRLEAGAIRELHWHNTPEWAYILKGTTQITAVDQNGRNYMANVGPGDLWYFPPGMPHSLQGTDANNEGSEFLLIFPDGTFDSSNQFMITDWLAHTPKDVIAKNFGVDISEFDRLPSHDLYIFPGVAPPLDAKAPEDPQGTVPLPFSFEFSKVKPTQYAGGTVKIADTRTFPIAKTISVAEVTVEPGAMRELHWHPTEDEWTYFIEGQARVTLFAGQSNAQTFDYQGGDIAYIPTAWGHYVENSGNTQLRFLEIFNSPLFEDVSLAQWIANTPPAIVKATLQLSDEVINTLNKQKAFVVASD

SEQ ID NO: 102 - protein, 437 aa, Artificial Sequence
  synthetic OXOX variant sequence
RPTENGPQIVIANNAGTYLPVPRGSGTKSSSAADATQTVPFASDDPNPRLWDIDTQDLSVVAPERGQLGAKILGPDNLPIDLQNADTLAPPTTDSGSVPNPKWPFALSHNTLYSGGWVRIQNDEVMPIAKAMAGVNMRLEAGAIRELHWHNTPEWAYILKGTTQITAVDQNGRNYMANVGPGDLWYFPPGMPHSLQGTDANNEGSEFLLIFPDGTFDASNQFMITDWLAHTPKDVIAKNFGVDISEFDRLPSHDLYIFPGVAPPLDATAPEDPQGTVPLPFSFEFSKVKPTQYAGGTVKIADTRTFPIAKTISVAEVTVEPGAMRELHWHPTEDEWTYFIEGQARVTLFAGQSNAQTFDYQGGDIAYIPTAWGHYVENSGNTQLRFLEIFNSPLFEDVSLAQWIANTPPAIVKATLQLSDEVINTLNKQKASVVASD

SEQ ID NO: 103 - protein, 437 aa, Artificial Sequence
  synthetic OXOX variant sequence
RPTENGPQIVIANNAGTYLPVPRGSGTKSSSAADATQTAPFASDDPNPRLWNIDTQDLSVVAPERGQLGAKILGPDNLPIDLQNADTLAPPTTDSGSVPNPKWPFALSHNTLYSGGWVRIQNDEVMPIAKAMAGVNMRLEAGAIRELHWHNTPEWAYILKGTTQITAVDQNGRNYMANVGPGDLWYFPEGMPHSLQGTDANNEGSEFLLIFPDGTFDASNQFMITDWLAHTPKDVIAKNFGVDISEFDRLPSHDLYIFPGVAPPLDAQAPEDPQGTVPLPYSFEFSKVKPTQYAGGTVKIADTRTFPIAKTISVAEVTVEPGAMRELHWHPTEDEWTYFIEGQARVTIFAGQSNAQTYDYQGGDIAYIPTAWGHYVENSGNTQLRFLEIFNSPLYEDVSLAQWIANTPPAIVKATLQLSDEVINTLNKQKAFVVASD

SEQ ID NO: 104 - protein, 435 aa, Artificial Sequence
  synthetic OXOX variant sequence
RPTENGPQIVIANNAGTYLPVLRGSGTKSSSAADATQTVPFASDDPNPRLWDIDTKNLEKATPERGQLGAEILGPDNLPLDIQNADTLAPPTTDSGSIPNPKWPFALSHNTLYSGGWVRIQNDEVMPIAKAMAGVNMRLEAGTIRELHWHNTPEWAYILKGTTQITAVDQNGRNYLANVGPGDLWYFPEGIPHSLQGTDANNEGSEFLLIFPDGTFDASNQFMITDWLAHTPKDVIAKNFGVDISEFDRLPSHDLYIFPGVAPPLDATAPEDPQGTIPLPYSFEFSKVKPTQYAGGTVKIADTRTFPIAKTISVAEVTVEPGAMRELHWHPTEDEWTFFIEGQARVTLFAGQSNAQTYDYQGGDIAYIPTAWGHYVENSGNTQLRFLEIFNSPLFEDVSLAQWIANTPPAIVKATLQLSDEVINTLNKQKASVVG

SEQ ID NO: 105 - protein, 435 aa, Artificial Sequence
  synthetic OXOX variant sequence
RPTENGPQIVIANNAGTYLPVLRGSGTKSSSAADATQTVPFASDDPNPRLWDMDTKNLEKVTPERGQLGAKILGPDNLPIDLQNADTLAPPTTDSGSIPNAKWPFALSHNTLYSGGWVRIQNDEVMPIAKAMAGVNMRLEAGTIRELHWHNTPEWAYILKGTTQITAVDQNGRNYLANVGPGDLWYFPEGIPHSLQGTNAADEGSEFLLIFPDGTFDSSNQFMITDWLAHTPKDVIAKNFGVDISEFDRLPSHDLYIFPGVAPPLDAKAPEDPQGTIPLPYSFEFSKVKPTQYAGGTVKIADTRTFPIAKTISVAEVTVEPGAMRELHWHPTEDEWTYFIEGQARVTLFAGESNAQTYDYQGGDIAYIPTAWGHYVENSGNTQLRFLEIFNSPLFEDVSLAQWIANTPPAIVKATLQLSDEVINTLNKQKASVVG

SEQ ID NO: 106 - protein, 435 aa, Artificial Sequence
  synthetic OXOX variant sequence
RPTENGPQIVIANNAGTYLPVLRGSGTESSSAAEPTETVPFASDDPNPRLWDIDTKNLEKVTPERGQLGAKILGPDNLPIDLQNADTLAPPTTDSGSIPNPKWPFALSHNTLYSGGWVRIQNDEVMPIAKAMAGVNMRLEAGTIRELHWHNTPEWAYILKGTTQITAVDQNGRNYLANVGPGDLWYFPEGIPHSLQGTDANNEGSEFLLIFPDGTFDASNQFMITDWLAHTPKDVIAKNFGVDISEFDRLPSHDLYIFPGVAPPLDATAPEDPQGTVPLPYSFEFSKVVPTQYAGGTVKIADTRTFPIAKTISVAEVTVEPGAMRELHWHPTEDEWTFFIEGQARVTIFAGESNAQTYDYQGGDIAYIPTAWGHYVENSGNTQLRFLEIFNSPLYEDVSLAQWIANTPRAIVKATLQLSDEVINTLNKQKASVVG

(19) United States
(12) Patent Application Publication
(10) Pub. No.: US 2011/0191903 A1
(43) Pub. Date: Aug. 4, 2011

(54) ENGINEERING PLANT RESISTANCE TO DISEASES CAUSED BY PATHOGENS

(75) Inventors: James Joseph English, San Leandro, CA (US); Xu Hu, Johnston, IA (US); Azalea Sukfun Ong, Castro Valley, CA (US); Daniel J. Thorpe, Johnston, IA (US); Gusui Wu, Grimes, IA (US)

(73) Assignee: Pioneer Hi-Bred International, Inc., Johnston, IA (US)

(21) Appl. No.: 12/982,059

(22) Filed: Dec. 30, 2010

Related U.S. Application Data
(60) Provisional application No. 61/291,721, filed on Dec. 31, 2009.

(51) Int. Cl. A01H 1/00; C12Q 1/26; C12N 15/82; C12N 9/02; C12N 5/10; C07H 21/04; A01H 5/00; A01H 5/10

(52) U.S. Cl. 800/279; 435/25; 435/468; 435/189; 435/419; 435/320.1; 536/23.2; 800/278; 800/298; 800/301

(57) ABSTRACT

Methods for identifying one or more amino acid substitutions in an oxalate oxidase (OXOX) variant polypeptide that confer maintained or increased OXOX activity are described herein. Methods and compositions for increasing a plant's resistance to a pathogen using the modified OXOX variant polypeptides are provided. Transformed plants, plant cell, tissues, seed, and expression vectors are also provided.

### CROSS-REFERENCE TO RELATED APPLICATIONS

This utility application claims the benefit U.S. Provisional Patent Application No. 61/291,721, filed Dec. 31, 2009, which is hereby incorporated herein in its entirety by reference.

### FIELD OF THE INVENTION

This invention relates to the genetic improvement of plants by the use of recombinant DNA techniques. Particularly, but not exclusively, the invention relates to the improvement of the tolerance of plants to pathogen attack.

### BACKGROUND OF THE INVENTION

Diseases of plants have caused an ongoing and constant problem in plant cultivation. The fungal pathogen, Sclerotinia sclerotiorum, in particular is said to cause disease in more than 400 plant species. Sclerotinia sclerotiorum appears to be among the most nonspecific, omnivorous, and successful of plant pathogens. (Purdy, L. H., Phytopathology 69: 875-880 (1979).

Sclerotinia infections in oil crops, for example, are considered the major disease problems of the crop yet little genetic resistance is currently available to breeding programs to combat the various forms of this fungal infection.

Oxalate (oxalic acid) is a diffusible toxin associated with various plant diseases, particularly those caused by fungi. While some leafy green vegetables, including spinach and rhubarb, produce oxalate as a nutritional stress factor, certain pathogens synthesize and export large amounts of oxalate to assist in the establishment and spread of the organism throughout infected hosts. Oxalate is used by pathogens to gain access into and subsequently throughout an infected plant. See for example, Mehta and Datta, J. Biol. Chem., 266: 23548-23553, and published PCT Application WO 92/14824 published in Sep. 3, 1992; Cessna, et al., Oxalic Acid, a Pathogenicity Factor for Sclerotinia sclerotiorum, Suppresses the Oxidative Burst of the Host Plant. (2000) Plant Cell, 12:2191-2200. Field crops such as sunflower, bean, canola, alfalfa, soybean, flax, safflower, peanut, clover, maize, sorghum, wheat, rice, as well as numerous vegetable crops, flowers, and trees are susceptible to oxalate-secreting pathogens. For example, fungal species including, but not limited to, Sclerotinia, Sclerotium, Aspergillus, Streptomyces, Penicillium, Pythium, Pacillus, Mycena, Leucostoma, Rhizoctonia and Schizophyllum use oxalic acid to provide an opportunistic route of entry into plants, causing serious damage to crops such as soybean, canola, sunflower and others susceptible to Sclerotinia infection. (Auclair, et al., Genetic interactions between Glycine max and Sclerotinia sclerotiorum using a straw inoculation method. (2004) Plant Dis. 88:891-895).

Enzymes that utilize oxalate as a substrate have been identified. These include oxalate oxidase (wheat oxalate oxidase is sometimes called germin) and oxalate decarboxylase. Oxalate oxidase catalyzes the conversion of oxalate to carbon dioxide and hydrogen peroxide. A gene encoding barley oxalate oxidase has been cloned from a barley root cDNA library and sequenced (See: PCT publication No. WO 92/14824, published in Sep. 3, 1992). A gene encoding wheat oxalate oxidase activity has been isolated and sequenced, and the gene has been introduced into a canola variety (PCT publication No. WO 92/15685 published in Sep. 17, 1992, Drawtewka-Kos, et al., J. Biol. Chem., 264 (9): 4896-4900 (1991)). Oxalate decarboxylase converts oxalate to carbon dioxide and formic acid. A gene encoding oxalate decarboxylase has been isolated from Collybia velutipes (now termed Flammulina velutipes) and the cDNA clone has been sequenced (WO 94/12622, published in Jun. 9, 1994). In addition, another oxalate decarboxylase gene has been isolated from Aspergillus phoenices (U.S. Pat. No. 6,297,425). A gene encoding oxalate oxidase from the dikaryotic white rot fungus Ceriporiopsis subvermispora were recently characterized and reported by Escutia et al. (Escutia et al., Cloning and sequencing of two Ceriporiopsis subvermispora bicupin oxalate oxidase allelic isoforms: implications for the reaction specificity of oxalate oxidases and decarboxylases. (2005)).

In many plants, attempted infection by avirulent pathogens triggers the activation of multiple defenses that may be accompanied by a hypersensitive response (HR) or collapse of host tissue around the site of pathogen penetration. A consequence of these responses is a restriction of pathogen spread within the host and frequently development of systemic acquired resistance (SAR) to subsequent infection by pathogens that may be taxonomically distant to the initial pathogen. For e.g., SAR induced by virus inoculation may be effective against subsequent attack by bacterial or fungal pathogens or vice versa. One of the earliest responses of the plant to infection is an oxidative burst which can be detected as an increased accumulation of superoxide (O₂) and/or hydrogen peroxide (H₂O₂). O₂ is very reactive and can form other reactive oxygen species, including hydroxyl radical (OH) and the more stable H₂O₂. H₂O₂ accumulation may trigger enhanced resistance responses in a number or ways: direct antimicrobial activity, act as a substrate for peroxidases associated with lignin polymerization and hence cell wall strengthening, via still to be determined mechanisms act as a signal for activation of expression of defense related genes, including those that result in stimulation of salicylic acid (SA) accumulation. SA is thought to act as an endogenous signal molecule that triggers expression of genes coding for several classes of pathogenesis-related proteins (PR proteins). Some of the PR proteins have antimicrobial enzymatic activities, such as glucanases and chitinases. The function of other PR proteins in defense still needs to be elucidated. Moreover, SA may potentiate the oxidative burst and thus act in a feedback loop enhancing its own synthesis. SA may also be involved in hypersensitive cell death by acting as an inhibitor of catalase, an enzyme that removes H₂O₂. H₂O₂ may trigger production of additional defense compounds such as phytoalexins, antimicrobial low molecular weight compounds. For a review on the role of the oxidative burst and SA please see Lamb, C. and Dixon, R. A., Ann. Rev. Physiol. Plant Mol. Biol., 48: 251-275 (1997). A high level of salicylic acid is associated with disease lesion mimic symptoms. Thus, the oxidative burst is the initial signal of a pathogen's attack, but one that is not permitted to be maintained by the plant. Even plants that are able to mount a defense are usually not immune to the disease. The pathogen is often able to inflict significant damage, although the plant may not die from the disease. Plants stressed because of pathogen damage are less likely to yield well and are often more susceptible to other types of pests. For these and other reasons, there is a need for the present invention.

### BRIEF SUMMARY OF THE INVENTION

Generally, it is an object of the invention to provide a method of identifying oxalate oxidase (OXOX) polypeptides that have maintained or increased OXOX activity. It is an object of the present invention to provide variant polynucleotides and polypeptides of OXOX's. It is an object of the present invention to provide transgenic plants comprising known fungal OXOX polynucleotides and polypeptides, OXOX variant polynucleotides and polypeptides of the present invention, or OXOX variant polynucleotides and polypeptides identified by methods of the invention. Yet another object of the present invention is to provide methods of increasing a plant's resistance to a pathogen. Therefore, in one aspect, the present invention relates to a method of identifying OXOX's that have maintained or increased OXOX activity.

The present invention also provides for an expression cassette comprising at least one known fungal OXOX polynucleotide encoding an OXOX polypeptide, or an OXOX variant polynucleotide encoding an OXOX variant polypeptide of the present invention, or OXOX variant polynucleotide encoding an OXOX polypeptide identified by a method of the present invention.

In another aspect, the present invention relates to an isolated OXOX variant polynucleotide that encodes any of the polypeptides of SEQ ID NOS: 18, 19, 21, 22, 23, 24, 25, 26, 27, 28, 29, 30, 31, 32, 33, 34, 35, 36, 37, 66, 67, 68, 69, 70, 71, 72, 73, 74, 75, 76, 77, 78, 79, 80, 81, 82, 83, 84, 85, 86, 87, 88, 89, 90, 91, 92, 100, 101, 102, 103, 104, 105, or 106; a polynucleotide having any of the sequences of SEQ ID NOS: 1, 2, 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 59, 60, 61, 62, 63, 64, 65, 93, 94, 95, 96, 97, 98, or 99; a polynucleotide having at least 30 nucleotides in length which hybridizes under stringent conditions to any of the former polynucleotides. In another aspect, the present invention includes a polynucleotide having at least 80% sequence identity to any of the sequences of SEQ ID NOS: 1, 2, 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 59, 60, 61, 62, 63, 64, 65, 93, 94, 95, 96, 97, 98, or 99. Provided herein in another aspect of the invention are isolated polynucleotides degenerate as a result of the genetic code for any of the OXOX's of the present invention. In another aspect, an isolated polynucleotide is complementary to a polynucleotide of any one of the OXOX's of the present invention. In another aspect, the present invention relates to an isolated polynucleotide that encodes an OXOX variant polypeptide that increases a plant's resistance to a pathogen.

In yet another aspect, the present invention relates to a transgenic plant including a recombinant expression cassette of a promoter functional in a plant operably linked to any of the isolated polynucleotides of the present invention. The present invention also provides for transgenic seed from the transgenic plant. In another aspect, the present invention is directed to a host cell transfected with the recombinant expression cassette of a promoter functional in a plant operably linked to a known fungal OXOX polynucleotide encoding an OXOX polypeptide or an OXOX variant polynucleotide encoding an OXOX variant polypeptide of the present invention.

In a further aspect, the present invention relates to an isolated OXOX variant polypeptide having OXOX activity. The OXOX variant polypeptide may have an amino acid sequence that has been substituted with at least one amino acid substitution at a position that corresponds to position 10, 19, 23, 26, 29, 35, 36, 38, 39, 40, 53, 54, 57, 58, 60, 61, 62, 63, 65, 68, 72, 79, 81, 83, 99, 102, 107, 115, 118, 124, 127, 131, 144, 148, 154, 159, 164, 166, 171, 174, 177, 181, 190, 192, 196, 200, 202, 203, 218, 219, 245, 259, 269, 278, 282, 287, 289, 290, 339, 349, 353, 359, 363, 373, 384, 387, 394, 395, 396, 399, 410, 425, 426, 427, 430, 433 or 436 of SEQ ID NO: 37, or additional amino acid residue at position 437 or 438 of SEQ ID NO:37; an amino acid sequence having at least 60%, 70%, 80%, 85%, 90%, 95%, 96%, 97%, 98%, 99% or 100% sequence identity to any of the amino acid sequences set forth in SEQ ID NOS: 18, 19, 21, 22, 23, 24, 25, 26, 27, 28, 29, 30, 31, 32, 33, 34, 35, 36, 37, 66, 67, 68, 69, 70, 71, 72, 73, 74, 75, 76, 77, 78, 79, 80, 81, 82, 83, 84, 85, 86, 87, 88, 89, 90, 91, 92, 100, 101, 102, 103, 104, 105, or 106; or a polypeptide encoded by any of the polynucleotides set forth in SEQ ID NOS: 1, 2, 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 59, 60, 61, 62, 63, 64, 65, 93, 94, 95, 96, 97, 98, or 99. In yet another aspect, the present invention relates to a transgenic plant of a recombinant expression cassette comprising a promoter functional in a plant operably linked to an isolated polynucleotide encoding a polypeptide that has an amino acid sequence that has at least 60%, 70%, 80%, 85%, 86%, 87%, 88%, 89%, 90%, 91%, 92%, 93%, 94%, 95%, 96%, 97%, 98%, 99%, or 100% sequence identity to any of the amino acid sequences set forth in SEQ ID NOS: 18, 19, 21, 22, 23, 24, 25, 26, 27, 28, 29, 30, 31, 32, 33, 34, 35, 36, 37, 66, 67, 68, 69, 70, 71, 72, 73, 74, 75, 76, 77, 78, 79, 80, 81, 82, 83, 84, 85, 86, 87, 88, 89, 90, 91, 92, 100, 101, 102, 103, 104, 105, or 106 and has OXOX activity or a known isolated polynucleotide encoding a fungal OXOX polypeptide having OXOX activity. The present invention also provides for transgenic seed from the transgenic plant. In another aspect, the present invention is directed to a host cell transfected with the recombinant expression cassette comprising a promoter functional in a plant operably linked to any of the isolated polynucleotides encoding polypeptides of the present invention or known fungal OXOX polynucleotides encoding polypeptides.

In a further aspect, the present invention relates to a method of modulating the level of OXOX protein or OXOX variant protein in a plant cell. In one aspect, the method includes transforming a plant cell with an OXOX variant polynucleotide of the present invention or known fungal OXOX polynucleotides operably linked to a promoter. The polynucleotide may be in sense or antisense orientation. The method further includes expressing the polynucleotide for an amount of time sufficient to modulate the level of OXOX protein or OXOX variant protein in the plant cell.

In another aspect, the present invention provides a method of modulating the level of OXOX protein or OXOX variant protein in a plant. The method includes stably transforming a plant cell with an OXOX variant polynucleotide of the present invention or known fungal OXOX polynucleotide, in sense or antisense orientation, operably linked to a promoter functional in a plant cell. The method includes regenerating the transformed plant cell into a transformed plant that expresses the OXOX variant polynucleotide or known fungal OXOX polynucleotide in an amount sufficient to modulate the level of OXOX variant protein or OXOX protein in the plant.

In another aspect, the present invention relates to a method of increasing a plant's resistance to a pathogen. In one aspect, the method includes introducing into plant cells a construct comprising a polynucleotide encoding an OXOX polypeptide of the present invention or known fungal polynucleotide encoding an OXOX polypeptide. The polynucleotide may be operably linked to a promoter functional in plant cells to yield transformed plant cells. The transformed plant cells are regenerated into a transgenic plant. The OXOX or variant OXOX is expressed in the cells of the transgenic plant at levels sufficient to increase OXOX activity. In one aspect, the OXOX is expressed in the cells of the transgenic plant at levels sufficient to increase a plant's resistance to a pathogen.

Other objects, features, advantages and aspects of the present invention will become apparent to those of skill from the following description. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure.

### BRIEF DESCRIPTION OF THE DRAWINGS

FIG. 1A. Schematic diagram for soybean transformation vector. OXOX-C (MOD1) SEQ ID NO:21 and OXOX-G (MOD1) SEQ ID NO:22 were cloned into plasmid vector expressing GM-ALS as selection marker. Mature proteins were fused with barley alpha amylase signal sequence (BAA SS) (MOD2) (SEQ ID NO:20) as N-terminal secreted signal. DMMV Promoter is the double enhancer domain of the Mirabilis Mosaic Caulimovirus. DMMV 5 UTR is 5′UTR of the DMMV Promoter. Maximum expression from the dMMV promoter requires 63 nucleotides downstream of the transcription start site (Plant Molecular Biology 40:771-782, 1999). BAA SS is the barley alpha-amylase signaling sequence (Knox, C., et al., Plant Molecular Biology 9:3-17, 1987) from Hordeum vulgare. NOS Terminator is the nopaline synthase (NOS) terminator that was originally extracted from Agrobacterium tumefaciens. GM-SAMS Promoter is the S-adenosyl-L-methionine synthetase (SAMS) promoter from Glycine max. GM-SAMS 5 UTR is the 5′ untranslated region of the soybean SAMS gene. GM-SAMS INTRON1 is Intron1 of GM-SAMS PRO (part of original GM-SAMS PRO feature). GM-SAMS 5 UTR (2) is the 5′ untranslated region of the soybean SAMS gene. GM-ALS (HRA) is the HRA allele of the acetolactate synthase coding sequence from Glycine max that is tolerant to ALS-inhibitor herbicides. GM-ALS (HRA) TERM is the terminator for the HRA acetolactate synthase coding sequence from Glycine max.

FIG. 1B. Schematic diagram of E. coli expression vector for OXOX shuffling. OXOX-C (MOD1) and OXOX-G (MOD1) were cloned into E. coli expression vector pET32 (Invitrogen) for enzyme kinetic, gene shuffling and mutagenesis studies. Mature proteins (SEQ ID NO:21 and SEQ ID NO:22) were fused 6×His-tag at C-terminus and a start codon (ATG) was added in front of the mature proteins.

FIG. 2. Fold improvement in OXOX activity at pH5.8 achieved by gene shuffling. Fold improvement in OXOX activity at pH5.8 is shown in terms of Kcat/Km compared with the polypeptide corresponding to sequence ALT1 (WT-Q7).

FIG. 3A-B. Sclerotinia T0 leaf disk bioassays and results. A single healthy leaf was collected in petri dish and two inoculation methods were used for disease evaluation as described in Example 7. Average disease scores of OXOX positive (open box) were compared with OXOX negative (black box) at 76 and 96 hours after inoculation using both plug inoculation method (FIG. 3A) and petiole inoculation method (FIG. 3B).

FIG. 4: Enhanced disease resistance of transgenic soybean plants constitutively expressing OXOX-C-ALT1. Disease responses to Sclerotinia infection were recorded 4 days after inoculation on a rating scale 1 to 9 as described in Example 8. Eight lines of transgenic plants expressing OXOX-C-ALT1, wild type (Jack), transformed Jack (4626.7.3 and 4626.7.4) and commercial tolerant line S1990 are shown.

FIG. 5: Alignment of OXOX polynucleotide sequences. Sequence alignment of OXOX-C-MOD1 and its variants, positions of nucleotide substitutions are indicated by asterisks.

FIG. 6: Sequence alignment of OXOX-C-MOD1 with 2-29 and consensus sequence obtained from alignment. The percent amino acid identity between OXOX-C-MOD1 and 2-29 is approximately 98%.

FIG. 7: Sequence alignment of OXOX-C-MOD1 with 4-128 and consensus sequence obtained from alignment. The percent amino acid identity between OXOX-C-MOD1 and 4-128 is approximately 94%.

FIG. 8: OXOX proteins digested in a standardized in vitro pepsin digestion assay. The reactions contained OXOX proteins in SGF with pepsin (10 U pepsin/ug protein in 0.084 N HCL, 35 mM NaCl) at 37 C for 0, 0.5, 1, 2, 5, 10, 30 and 60 minutes in lane 1 to 8 of 10-20% polyacrylamide tricine gels as described in Example 9.

### BRIEF DESCRIPTION OF THE SEQUENCES

The application provides details of OXOX sequences and OXOX variants and others as shown in Tables 1-3 below.

TABLE 1

| SEQ ID | Polynucleotide (pnt) | | |
|---|---|---|---|
| NO: | or polypeptide (ppt) | Length | Identification |
| 1 | pnt | 1368 | OXOX-C |
| | | | cDNA full length assembled from AJ746414 |
| | | | and AJ563659 |
| 2 | pnt | 1386 | OXOX-G |
| | | | AJ746412 cDNA |
| 3 | pnt | 72 | Barley alpha amylase signal sequence BAA |
| | | | SS (MOD2) |
| 4 | pnt | 1308 | OXOX-C (MOD1) |
| | | | Synthetic gene with soybean preferred codon |
| 5 | pnt | 1326 | OXOX-G (MOD1) |
| | | | Synthetic gene with soybean preferred codon |
| 6 | pnt | 1308 | OXOX-C-MOD1-ALT1 |
| | | | glycosylation minus variant (WT-Q7) from |
| | | | SEQ ID NO: 4 |
| 7 | pnt | 1308 | OXOX-C- MOD1-ALT2 |
| | | | glycosylation minus variant (glyc-) from SEQ |
| | | | ID NO: 4 |
| 8 | pnt | 1308 | OXOX-C- MOD1-ALT3 |
| | | | 1st round shuffled variant (1-8) |
| | | | from OXOX-C-ALT1 (SEQ ID NO: 6) |
| 9 | pnt | 1308 | OXOX-C- MOD1-ALT4 |
| | | | 1st round shuffled variant (1-23) from |
| | | | OXOX-C-ALT1 (SEQ ID NO: 6) |
| 10 | pnt | 1308 | OXOX-C- MOD1-ALT5 |
| | | | 2nd round shuffled variant (2-6) |
| 11 | pnt | 1308 | OXOX-C- MOD1-ALT6 |
| | | | 3rd round shuffled variant (3-21) |
| 12 | pnt | 1308 | OXOX-C- MOD1-ALT7 |
| | | | 3rd round shuffled variant (3-25) |
| 13 | pnt | 1314 | FG10 |
| | | | 3rd round shuffled variant (FG10) |
| 14 | pnt | 1308 | FG23 |
| | | | 3rd round shuffled variant (FG23) |
| 15 | pnt | 1308 | 3-10 |
| | | | 3rd round shuffled variant (3-10) |
| 16 | pnt | 1308 | 3-20 |
| | | | 3rd round shuffled variant (3-20) |
| 17 | pnt | 1308 | 3-26 |
| | | | 3rd round shuffled variant (3-26) |
| 18 | ppt | 455 | OXOX-C |
| | | | Translated cDNA full length assembled from |
| | | | AJ746414 and AJ563659 |
| 19 | ppt | 461 | OXOX-G |
| | | | Translated AJ746412 OXOX-G cDNA |
| 20 | ppt | 24 | Barley alpha amylase signal sequence |
| 21 | ppt | 435 | OXOX-C- MOD1 |
| | | | Synthetic gene with soybean preferred codon |
| 22 | ppt | 441 | OXOX-G- MOD1 |
| | | | Synthetic gene with soybean preferred codon |
| 23 | ppt | 435 | OXOX-C- MOD1-ALT1 |
| | | | glycosylation minus variant |
| 24 | ppt | 435 | OXOX-C- MOD1-ALT2 |
| | | | glycosylation minus variant |
| 25 | ppt | 435 | OXOX-C- MOD1-ALT3 |
| | | | 1st round shuffled variant from OXOX-C- |
| | | | MOD1ALT1 (SEQ ID NO: 6) |
| 26 | ppt | 435 | OXOX-C- MOD1-ALT4 |
| | | | 1st round shuffled variant from OXOX-C- |
| | | | MOD1ALT1 (SEQ ID NO: 6) |
| 27 | ppt | 435 | OXOX-C- MOD1-ALT5 |
| | | | 2nd round shuffled variant |
| 28 | ppt | 435 | OXOX-C- MOD1-ALT6 |
| | | | 3rd round shuffled variant |
| 29 | ppt | 435 | OXOX-C- MOD1-ALT7 |
| | | | 3rd round shuffled variant |
| 30 | ppt | 437 | 3-FG10 |
| | | | 3rd round shuffled variant |
| 31 | ppt | 435 | 3-FG23 |
| | | | 3rd round shuffled variant |
| 32 | ppt | 435 | 3-10 |
| | | | 3rd round shuffled variant |
| 33 | ppt | 435 | 3-20 |
| | | | rd round shuffled variant |
| 34 | ppt | 435 | 3-26 |
| | | | 3rd round shuffled variant |
| 35 | ppt | 436 | methionine added to beginning of amino acid |
| | | | sequence of OXOX-C-MOD1-ATL1 (SEQ ID NO: 23) |
| 36 | ppt | 436 | methionine added to beginning of amino acid |
| | | | sequence of OXOX-C-MOD1-ATL2 (SEQ ID NO: 24) |
| 37 | ppt | 436 | methionine added to beginning of amino acid |
| | | | sequence of OXOX-C-MOD1 (SEQ ID NO: 21) |
| 38 | pnt | 10 | Sequence in promoter of barley beta-1,3- |
| | | | glucanase |
| 39 | pnt | 1308 | 2-6-2 |
| | | | 4^{th} round shuffled variant |
| 40 | pnt | 1308 | 2-7 |
| | | | 4^{th} round shuffled variant |
| 41 | pnt | 1308 | 2-27 |
| | | | 4^{th} round shuffled variant |
| 42 | pnt | 1308 | 2-29 |
| | | | 4^{th} round shuffled variant |
| 43 | pnt | 1308 | 3F3 |
| | | | 4^{th} round shuffled variant |
| 44 | pnt | 1308 | 4-15 |
| | | | 4^{th} round shuffled variant |
| 45 | pnt | 1308 | 4-19 |
| | | | 4^{th} round shuffled variant |
| 46 | pnt | 1314 | 4-23 |
| | | | 4^{th} round shuffled variant |
| 47 | pnt | 1314 | 4-37 |
| | | | 4^{th} round shuffled variant |
| 48 | pnt | 1308 | 4-43 |
| | | | 4^{th} round shuffled variant |
| 49 | pnt | 1308 | 4-53 |
| | | | 4^{th} round shuffled variant |
| 50 | pnt | 1314 | 4-65 |
| | | | 4^{th} round shuffled variant |
| 51 | pnt | 1308 | 4-67 |
| | | | 4^{th} round shuffled variant |
| 52 | pnt | 1314 | 4-76 |
| | | | 4^{th} round shuffled variant |
| 53 | pnt | 1308 | 4-78 |
| | | | 4^{th} round shuffled variant |
| 54 | pnt | 1308 | 4-79 |
| | | | 4^{th} round shuffled variant |
| 55 | pnt | 1308 | 4-83 |
| | | | 4^{th} round shuffled variant |
| 56 | pnt | 1308 | 4-84 |
| | | | 4^{th} round shuffled variant |
| 57 | pnt | 1314 | 4-85 |
| | | | 4^{th} round shuffled variant |
| 58 | pnt | 1308 | 4-87 |
| | | | 4^{th} round shuffled variant |
| 59 | pnt | 1308 | 4-91 |
| | | | 4^{th} round shuffled variant |
| 60 | pnt | 1308 | 4-96 |
| | | | 4^{th} round shuffled variant |
| 61 | pnt | 1308 | 4-113 |
| | | | 4^{th} round shuffled variant |
| 62 | pnt | 1314 | 4-118 |
| | | | 4^{th} round shuffled variant |
| 63 | pnt | 1308 | 4-124 |
| | | | 4^{th} round shuffled variant |
| 64 | pnt | 1314 | 4-128 |
| | | | 4^{th} round shuffled variant |
| 65 | pnt | 1308 | FG15 |
| | | | 4^{th} round shuffled variant |
| 66 | ppt | 435 | 2-6-2 |
| | | | 4^{th} round shuffled variant |
| 67 | ppt | 435 | 2-7 (BAA-OXOX-C-MOD1- ALT14) |
| | | | 4^{th} round shuffled variant |
| 68 | ppt | 435 | 2-27 (BAA-OXOX-C-MOD1- ALT15) |
| | | | 4^{th} round shuffled variant |
| 69 | ppt | 435 | 2-29 |
| | | | 4^{th} round shuffled variant |
| 70 | ppt | 435 | 3F3 (BAA-OXOX-C-MOD1- ALT9) |
| | | | 4^{th} round shuffled variant |
| 71 | ppt | 435 | 4-15 |
| | | | 4^{th} round shuffled variant |
| 72 | ppt | 435 | 4-19 |
| | | | 4^{th} round shuffled variant |
| 73 | ppt | 437 | 4-23 |
| | | | 4^{th} round shuffled variant |
| 74 | ppt | 437 | 4-37 |
| | | | 4^{th} round shuffled variant |
| 75 | ppt | 435 | 4-43 |
| | | | 4^{th} round shuffled variant |
| 76 | ppt | 435 | 4-53 |
| | | | 4^{th} round shuffled variant |
| 77 | ppt | 437 | 4-65 |
| | | | 4^{th} round shuffled variant |
| 78 | ppt | 435 | 4-67 |
| | | | 4^{th} round shuffled variant |
| 79 | ppt | 437 | 4-76 |
| | | | 4^{th} round shuffled variant |
| 80 | ppt | 435 | 4-78 |
| | | | 4^{th} round shuffled variant |
| 81 | ppt | 435 | 4-79 |
| | | | 4^{th} round shuffled variant |
| 82 | ppt | 435 | 4-83 |
| | | | 4^{th} round shuffled variant |
| 83 | ppt | 435 | 4-84 (BAA-OXOX-C-MOD1- ALT13) |
| | | | 4^{th} round shuffled variant |
| 84 | ppt | 437 | 4-85 (BAA-OXOX-C-MOD1- ALT10) |
| | | | 4^{th} round shuffled variant |
| 85 | ppt | 435 | 4-87 |
| | | | 4^{th} round shuffled variant |
| 86 | ppt | 435 | 4-91 |
| | | | 4^{th} round shuffled variant |
| 87 | ppt | 435 | 4-96 |
| | | | 4^{th} round shuffled variant |
| 88 | ppt | 435 | 4-113 (BAA-OXOX-C-MOD1- ALT11) |
| | | | 4^{th} round shuffled variant |
| 89 | ppt | 437 | 4-118 (BAA-OXOX-C-MOD1- ALT12) |
| | | | 4^{th} round shuffled variant |
| 90 | ppt | 435 | 4-124 |
| | | | 4^{th} round shuffled variant |
| 91 | ppt | 437 | 4-128 |
| | | | 4^{th} round shuffled variant |
| 92 | ppt | 435 | FG15 (BAA-OXOX-C-MOD1- ALT8) |
| | | | 4^{th} round shuffled variant |
| 93 | pnt | 1308 | 183-FG15 |
| | | | 5th round shuffled variant |
| 94 | pnt | 1314 | 4G |
| | | | 4th round shuffled variant |
| 95 | pnt | 1314 | 7G |
| | | | 5th round shuffled variant |
| 96 | pnt | 1314 | 8G |
| | | | 5th round shuffled variant |
| 97 | pnt | 1308 | FG-B5 |
| | | | 3^{rd} round shuffled variant |
| 98 | pnt | 1308 | FG-E5 |
| | | | 3rd round shuffled variant |
| 99 | pnt | 1308 | FG-G6 |
| | | | 3rd round shuffled variant |
| 100 | ppt | 435 | 183-FG15 |
| | | | 5th round shuffled variant |
| 101 | ppt | 437 | 4G |
| | | | 4th round shuffled variant |
| 102 | ppt | 437 | 7G |
| | | | 5th round shuffled variant |
| 103 | ppt | 437 | 8G |
| | | | 5th round shuffled variant |
| 104 | ppt | 435 | FG-B5 |
| | | | 3rd round shuffled variant |
| 105 | ppt | 435 | FG-E5 |
| | | | 3rd round shuffled variant |
| 106 | ppt | 435 | FG-G6 |
| | | | 3rd round shuffled variant |

Table 2 shows amino acid substitutions in OXOX that are believed to be functional and may influence OXOX activity, e.g., specificity or digestibility. In some cases, for example, three glycosylation sites were eliminated by substitution. The start codon ATG (methionine) was added to OXOX mature protein for E. coli expression.

TABLE 2

| | | | | Identification and position |
|---|---|---|---|---|
| | | | | of amino acid substitution |
| SEQ ID | | | | in OXOX relative to |
| NO: | Name | Polypeptide (ppt) | Length | SEQ ID NO. 37 |
| 35 | methionine added to | (M)RPTENGPQIVIANNAGTYLPVL | 436 | T60I; T384Q; S430Q |
| | beginning of amino | RGSGTKSSSAADATQTVPFASDDPN | | |
| | acid sequence of | PRLWDIDTKNLIKVTPERGQLGAKI | | |
| | OXOX-C-MOD1- | LGPDNLPIDLQNADTLAPPTTDSGS | | |
| | ATL1 | IPNPKWPFALSHNTLYSGGWVRIQN | | |
| | (SEQ ID NO: 23) | DEVMPIAKAMAGVNMRLEAGAIREL | | |
| | | HWHNTPEWAYILKGTTQITAVDQNG | | |
| | | RNYLANVGPGDLWYFPEGMPHSLQG | | |
| | | TDANNEGSEFLLIFPDGTFDSSNQF | | |
| | | MITDWLAHTPKDVIAKNFGVDISEF | | |
| | | DRLPSHDLYIFPGVAPPLDAKAPED | | |
| | | PQGTIPLPYSFEFSKVKPTQYAGGT | | |
| | | VKIADIRTFPIAKTISVAEVTVEPG | | |
| | | AMRELHWHPTEDEWTFFIEGQARVT | | |
| | | IFAGQSNAQTYDYQGGDIAYIPTAW | | |
| | | GHYVENSGNTQLRFLEIFNSPLFED | | |
| | | VSLAQWIANTPPAIVKATLQLSDEV | | |
| | | INTLNKQKAFVVG | | |
| | | (SEQ ID NO: 35) | | |
| 36 | methionine added to | (M)RPTENGPQIVIANNAGTYLPVL | 436 | T60R; T384V; S430D |
| | beginning of amino | RGSGTKSSSAADATQTVPFASDDPN | | |
| | acid sequence of | PRLWDIDTKNLRKVTPERGQLGAKI | | |
| | OXOX-C-MOD1- | LGPDNLPIDLQNADTLAPPTTDSGS | | |
| | ATL2 | IPNPKWPFALSHNTLYSGGWVRIQN | | |
| | (SEQ ID NO: 24) | DEVMPIAKAMAGVNMRLEAGAIREL | | |
| | | HWHNTPEWAYILKGTTQITAVDQNG | | |
| | | RNYLANVGPGDLWYFPEGMPHSLQG | | |
| | | TDANNEGSEFLLIFPDGTEDSSNQF | | |
| | | MITDWLAHTPKDVIAKNFGVDISEF | | |
| | | DRLPSHDLYIFPGVAPPLDAKAPED | | |
| | | PQGTIPLPYSFEFSKVKPTQYAGGT | | |
| | | VKIADTRTFPIAKTISVAEVTVEPG | | |
| | | AMRELHWHPTEDEWTFFIEGQARVT | | |
| | | IFAGQSNAQTYDYQGGDIAYIPTAW | | |
| | | GHYVENSGNTVLRFLEIFNSPLFED | | |
| | | VSLAQWIANTPPAIVKATLQLSDEV | | |
| | | INTLNKDKAFVVG | | |
| | | (SEQ ID NO: 36) | | |

*The amino acid sequence of SED ID NO: 37 contains methionine (M) in front of mature protein of OXOX-C- MOD1 (SEQ ID NO 21), as the E. coli vector started with M (methionine).

Table 3 shows amino acid substitutions in OXOX that are believed to be functional and may influence OXOX activity. For example, the OXOX variants may have increased OXOX activity, specificity or digestibility. Numbering is relative to mature OXOX-C sequence that was expressed in E. coli. Amino acid substitutions relative to OXOX-C-ALT1 (SEQ ID NO:35).

TABLE 3

| | | | Identification and position of | |
|---|---|---|---|---|
| SEQ ID | | | amino acid substitution in OXOX | # of |
| NO. | Name | Length | of relative to SEQ ID NO: 35 | substitutions |
| 24 | OXOX- | 435 | I60R, Q384V, Q430D | 3 |
| | C-ALT2 | | | |
| 25 | OXOX- | 435 | I54M, I99V, W118Y, F259Y, | 7 |
| | C-ALT3 | | I278V, F339Y, F396Y | |
| 26 | OXOX- | 435 | W118Y, I166V, F259Y, I278V, | 7 |
| | C-ALT4 | | Y282F, F339Y, F396Y | |
| 27 | OXOX- | 435 | I54M, I60E, I99V, W118Y, F259Y, | 9 |
| | C-ALT5 | | I278V, F339Y, Y359F, F396Y | |
| 28 | OXOX- | 435 | L23P, I60E, T63Q, I99V, L177M, | 11 |
| | C-ALT6 | | L196I, F259Y, I278V, F339Y, | |
| | (3-21) | | F387Y, F396Y | |
| 29 | OXOX- | 435 | L23P, I54M, I60E, I99V, W118Y, | 15 |
| | C-ALT7 | | P154A, I159V, L177M, D218N, | |
| | (3-25) | | F259Y, I278V, F339Y, Y359F, | |
| | | | G363A, F396Y | |
| 30 | 3-FG10 | 437 | D53N, I60E, E65Q, K72E, S115T, | 15 |
| | (mature) | | Q171E, R174K, M192V, I278V, | |
| | | | Y282F, Y359F, V399I, G436A plus | |
| | | | S437, D438 | |
| 31 | 3-FG23 | 435 | D53N, K57Q, N58D, I60S, K61V, | 18 |
| | | | T63A, Q68P, I99V, D124N, | |
| | | | M127L, G181S, M192I, S219A, | |
| | | | I278V, W373Y, N425D, T426S, | |
| | | | F433S | |
| 32 | 3-10 | 435 | Y19H, L23P, I54M, I60E, I99V, | 16 |
| | | | W118Y, K131T, L177M, E190P, | |
| | | | F259Y, K269Q, F339Y, Y359F, | |
| | | | P394D, L395V, F396Y | |
| 33 | 3-20 | 435 | I54M, I60E, T63Q, A107S, | 11 |
| | | | W118Y, L196I, F259Y, I278V, | |
| | | | F339Y, Y359F, F396Y | |
| 34 | 3-26 | 435 | L23P, I99V, W118Y, L148M, | 13 |
| | | | I159V, Q171E, L177M, F259V, | |
| | | | I278V, F339Y, Y359F, F387Y, | |
| | | | L427F, F433L | |
| 66 | 2-6-2 | 435 | I10V, I60E, F259Y, I278V, | 8 |
| | | | V289A, F339Y, Y359F, F396Y | |
| 67 | 2-7 | 435 | D53G, I60E, I99V, F259Y, I278V, | 9 |
| | | | Y282F, F339Y, Y359F, F396Y | |
| 68 | 2-27 | 435 | I10T, I60E, I99V, F259Y, I278V, | 9 |
| | | | Y282F, F339Y, Y359F, F396Y | |
| 69 | 2-29 | 435 | I60E, I99V, F259Y, I278V, Y282F, | 8 |
| | | | F339Y, Y359F, F396Y | |
| 70 | 3F3 | 435 | I60E, K72E, L79I, W118Y, M192I, | 9 |
| | | | I278V, F339Y, F396Y, F433S | |
| 71 | 4-15 | 435 | Y19H, L23P, K57Q, N58D, I60S, | 21 |
| | | | K61V, T63A, Q68P, W118Y, | |
| | | | K131T, L177M, E190P, S219A, | |
| | | | I245T, K269Q, I278V, F339Y, | |
| | | | Y359F, P394D, L395V, F396Y | |
| 72 | 4-19 | 435 | Y19H, L23P, I60E, I99V, W118Y, | 17 |
| | | | L177M, G181S, E190P, I278V, | |
| | | | Y359F, W373Y, P394D, L395V, | |
| | | | F396Y, N425D, T426S, F433S | |
| 73 | 4-23 | 437 | L23P, D53N, K57Q, N58D, I60S, | 19 |
| | | | K61V, T63A, Q68P, I99V, | |
| | | | W118Y, L148M, E190P, I278V, | |
| | | | F339Y, Y359F, F396Y, G436A | |
| | | | plus S437, D438 | |
| 74 | 4-37 | 437 | Y19H, L23P, I54M, I60E, I99V, | 17 |
| | | | W118Y, M127L, K131T, L177M, | |
| | | | E190P, I278V, Y359F, G363A, | |
| | | | F396Y, G436A plus S437, D438 | |
| 75 | 4-43 | 435 | I54M, I60E, Q68P, I99V, W118Y, | 13 |
| | | | L148M, I159V, M192V, I278V, | |
| | | | F339Y, Y359F, G363A, F433S | |
| 76 | 4-53 | 435 | Y19H, L23P, I54M, I60E, I99V, | 16 |
| | | | L148M, I159V, Q171E, L177M, | |
| | | | E190P, I278V, Y282F, P394D, | |
| | | | L395V, F396Y, F433S | |
| 77 | 4-65 | 437 | I54M, I60E, W118Y, L177M, | 16 |
| | | | G181S, I278V, Y282F, F339Y, | |
| | | | Y359F, G363A, F396Y, N425D, | |
| | | | T426S, G436A plus S437, D438 | |
| 78 | 4-67 | 435 | K57Q, N58D, I60S, K61V, T63A, | 21 |
| | | | Q68P, I99V, W118Y, D124N, | |
| | | | M127L, I159V, Q171E, L177M, | |
| | | | M192V, K269Q, I278V, F339Y, | |
| | | | Y359F, N425D, T426S, F433S | |
| 79 | 4-76 | 437 | L23P, D53N, S115T, M127L, | 17 |
| | | | L148M, Q171E, L177M, E190P, | |
| | | | F259Y, K269Q, I278V, F339Y, | |
| | | | Y359F, L427F, G436A plus S437, | |
| | | | D438 | |
| 80 | 4-78 | 435 | L23P, I54M, I60E, E65Q, K72E, | 17 |
| | | | I99V, K131T, Q171E, R174K, | |
| | | | M192V, K269Q, I278V, F339Y, | |
| | | | Y359F, P394D, L395V, F396Y, | |
| 81 | 4-79 | 435 | D53N, K57Q, N58D, I60S, K61V, | 14 |
| | | | T63A, Q68P, I99V, W118Y, | |
| | | | L148M, L177M, I278V, Y359F, | |
| | | | G363A, | |
| 82 | 4-83 | 435 | Y19H, L23P, I54M, I60E, K72E, | 13 |
| | | | W118Y, L148M, P154Q, I278V, | |
| | | | F339Y, Y359F, G363A, F396Y | |
| 83 | 4-84 | 435 | Y19H, L23P, I54M, I60E, E65Q, | 13 |
| | | | K72E, W118Y, G181S, I278V, | |
| | | | F339Y, Y359F, G363A, F396Y | |
| 84 | 4-85 | 437 | L23P, V40A, I54M, I60E, T63Q, | 15 |
| | | | W118Y, L177M, E190P, K269Q, | |
| | | | I278V, F339Y, L427F, G436A | |
| | | | plus S437, D438 | |
| 85 | 4-87 | 435 | T39A, I54M, I60E, K72E, I99V, | 15 |
| | | | D124N, M127L, L148M, R174K, | |
| | | | E190P, K269Q, I278V, Y359F, | |
| | | | N425D, T426S | |
| 86 | 4-91 | 435 | L23P, I54M, I60E, W118Y, | 9 |
| | | | L148M, L177M, F259V, I278V, | |
| | | | F339Y | |
| 87 | 4-96 | 435 | Y19H, L23P, I54M, I60E, W118Y, | 13 |
| | | | K131T, I159V, L177M, M192V, | |
| | | | F259Y, I278V, S287C, Y359F | |
| 88 | 4-113 | 435 | Y19H, L23P, S26T, I54M, I60E, | 19 |
| | | | D124N, M127L, K131T, I159V, | |
| | | | L177M, E190P, S219A, K269Q, | |
| | | | I278V, F339Y, Y359F, F387Y, | |
| | | | V399I, F433S | |
| 89 | 4-118 | 437 | L23P, D53N, K57Q, N58D, I60S, | 18 |
| | | | K61V, T63A, S115T, P154A, | |
| | | | L177M, E190P, S219A, F259Y, | |
| | | | K269Q, Y359F, G436A plus S437, | |
| | | | D438 | |
| 90 | 4-124 | 435 | L23P, I54M, I60E, E65Q, K72E, | 14 |
| | | | S115T, L148M, G181S, S219A, | |
| | | | K269Q, I278V, F339Y, Y359F, | |
| | | | F396Y | |
| 91 | 4-128 | 437 | L23P, D53N, K57Q, N58D, I60S, | 23 |
| | | | K61V, T63A, A107S, W118Y, | |
| | | | P154A, I159V, L177M, I278V, | |
| | | | Y282F, F339Y, Y359F, G363A, | |
| | | | P394D, L395V, F396Y, G436A | |
| | | | plus S437, D438 | |
| 92 | FG15 | 435 | D35E, A36P, Q38E, K57Q, N58D, | 19 |
| | | | I60S, K61V, T63A, I99V, D200N, | |
| | | | N202A, N203D, K269Q, I278V, | |
| | | | Y282F, K290V, F339Y, I349L, | |
| | | | F433S | |
| 100 | 183-FG15 | 435 | D35E, A36P, Q38E, K57Q, N58D, | 20 |
| | | | I60S, K61V, T63A, I99V, T164D, | |
| | | | D200N, N202A, N203D, K269Q, | |
| | | | I278V, Y282F, K290V, F339Y, | |
| | | | I349L, F433S | |
| 101 | 4G | 437 | L23P, D53N, K57Q, N58D, I60S, | 18 |
| | | | K61V, T63A, W118Y, L177M, | |
| | | | E190P, I278V, Y282F, F339Y, | |
| | | | I349L, Y359F, G436A plus S437, | |
| | | | D438 | |
| 102 | 7G | 437 | L23P, K57Q, N58D, I60S, K61V, | 20 |
| | | | T63A, I99V, L177M, E190P, | |
| | | | S219A, K269T, I278V, Y282F, | |
| | | | F339Y, I349L, Y359F, F433S, | |
| | | | G436A plus S437, D438 | |
| 103 | 8G | 437 | L23P, V40A, D53N, K57Q, N58D, | 18 |
| | | | I60S, K61V, T63A, I99V, L177M, | |
| | | | S219A, K269Q, I278V, F339Y, | |
| | | | F396Y, G436A plus S437, D438 | |
| 104 | FG-B5 | 435 | I60E, V62A, K72E, I81L, L83I, | 10 |
| | | | A144T, M192I, S219A, K269T, | |
| | | | F433S | |
| 105 | FG-E5 | 435 | I54M, I60E, P102A, A144T, | 12 |
| | | | M192I, D200N, N202A, N203D, | |
| | | | F339Y, I349L, Q353E, F433S | |
| 106 | FG-G6 | 435 | K29E, D35E, A36P, Q38E, I60E, | 15 |
| | | | A144T, M192I, S219A, K269T, | |
| | | | I278V, K290V, Q353E, F396Y, | |
| | | | P410R, F433S | |

### DETAILED DESCRIPTION OF THE INVENTION

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the invention.

In the description that follows, a number of terms are used extensively. The following definitions are provided to facilitate understanding of the invention.

A structural gene is a region of DNA having a sequence that is transcribed into messenger RNA (mRNA) that is then translated into a sequence of amino acids characteristic of a specific polypeptide. Structural genes also include gene encoding RNA products directly such as genes encoding transfer RNA (tRNA).

As used herein promoter includes reference to a region of DNA upstream from the start of transcription and involved in recognition and binding of RNA polymerase and other proteins to initiate transcription. A plant promoter is a promoter capable of initiating transcription in plant cells. Exemplary plant promoters include, but are not limited to, those that are obtained from plants, plant viruses, and bacteria which comprise genes expressed in plant cells such Agrobacterium or Rhizobium. Examples are promoters that preferentially initiate transcription in certain tissues, such as leaves, roots, seeds, fibers, xylem vessels, tracheids, or sclerenchyma. Such promoters are referred to, as tissue preferred. A cell type specific promoter primarily drives expression in certain cell types in one or more organs, for example, vascular cells in roots or leaves. An inducible promoter is a promoter that is under environmental control. Examples of environmental conditions that may effect transcription by inducible promoters include anaerobic conditions or the presence of light. Another type of promoter is a developmentally regulated promoter, for example a promoter that drives expression during pollen development. Tissue preferred, cell type specific, developmentally regulated, and inducible promoters constitute the class of non-constitutive promoters. A constitutive promoter is a promoter that is active under most environmental conditions.

An element is a region of DNA having a sequence that is involved in the regulation of gene expression. Examples of elements include terminators, introns, polyadenylation sequences, nucleic acid sequences encoding signal peptides which permit localization within a plant cell or secretion of the protein from the cell, or as in the present invention a nucleic acid sequence that regulates transcription in response to an inducer or the signal produced in response to an inducer.

An enhancer is a DNA regulatory region that can increase the efficiency of transcription, and may or may not be independent of the distance or orientation of the enhancer relative to the start site of transcription.

Complementary DNA (cDNA) is a single-stranded DNA molecule that is formed from an mRNA template by the enzyme reverse transcriptase. Typically, a primer complementary to portions of mRNA is employed for the initiation of reverse transcription. hose skilled in the art also use the term “cDNA” to refer to a double-stranded DNA molecule consisting of such a single-stranded DNA molecule and its complementary DNA strand.

An endogenous gene refers in the present description to a gene that is in its native form and has not been modified in composition or genomic locus.

As used herein, the term “plant” includes reference to whole plants, plant organs (e.g., leaves, stems, roots, etc.), seeds and plant cells and progeny of same. As used herein, the term plant also includes plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides. The class of plants which can be used in the methods of the invention is generally as broad as the class of higher plants amenable to transformation techniques, including both monocotyledonous and dicotyledonous plants. A particularly preferred plant is Zea mays or soybean.

T0 refers to the initial transgenic shoot or plant recovered from the transformation and cultural protocols whether the plant is maintained in vitro or established in soil. The T1 generation are those plants resulting from seed recovered from, most commonly, self pollinated T0 plants, or from seed obtained by crosses with other lines where the T0 candidate is either the male or female parent. The T2 generation is the material obtained from T1 selfings or crosses.

The term oxidase as used in this application refers to an enzyme capable of generating hydrogen peroxide or any reactive oxygen species.

A pathogen refers to any organism responsible for disease and/or damage to a plant. As used herein, the term “pathogen” is intended to include fungi, bacteria, nematodes, viruses, parasitic weeds, pests, biological agents, disease-producing microorganisms, toxic biological products, and organic biocides that can cause death or injury to plants. For the present invention, pests include but are not limited to insects, fungi, bacteria, nematodes, viruses or viroids, parasitic weeds, and the like.

For the purposes of the present invention, a plant that is tolerant to a pathogen or other stress is one that is able to withstand a pathogen attack or stressful conditions better than the wild type plant, but will usually succumb to infection and/or die under conditions other than very light disease or stress pressure. A resistant plant is a plant having the ability to exclude or overcome the growth or effects of a pathogen or stress except under extremely high disease or stress pressure. An immune plant is one capable of complete disease resistance, with no reaction of plant tissue to a potential pathogen.

As used herein, the term “fungal oxalate oxidase” or “fungal OXOX” includes but is not limited to known OXOX sequences, the sequences or polymorphisms disclosed herein, their conservatively modified variants, regardless of source and any other variants which retain or increase the biological properties of the OXOX, for example, OXOX activity as disclosed herein.

As used herein, the term “oxalate oxidase variants” or “OXOX variants” includes but is not limited to the sequences or polymorphisms disclosed herein, their conservatively modified variants, regardless of source and any other variants which retain or increase the biological properties of the OXOX, for example, OXOX activity as disclosed herein.

As used herein, the term “digestibilty” refers to how resistant or susceptible a protein is to being digested or broken down, for example, a protein's digestive stability when subjected to a protease or enzyme such as pepsin. In some examples, digestibilty of the OXOX variant polypeptide or known fungal OXOX polypeptide is increased by at least 1%, 5%, 10%, 20%, 30%, 50%, 50%, 60%, 70%, 80%, or 90% relative to a control protein, for example, having increased digestibility or exhibiting greater susceptibility to being digested as compared to the digestibility of a plant OXOX protein such as a wheat OXOX protein or a wild type fungal OXOX protein. In some examples, digestibilty of the OXOX variant polypeptide or known fungal OXOX polypeptide is decreased by at least 1%, 5%, 10%, 20%, 30%, 50%, 50%, 60%, 70%, 80%, or 90% relative to a control protein, for example, having decreased digestibility or exhibiting greater resistance to being digested as compared to the digestibility of a plant OXOX protein such as a wheat OXOX protein. See also Example 9 describing a synthetic gastric fluid (SGF) assay an exemplary in vitro standard technique that may be used to determine digestibility of a protein. Those in the art will be familiar with other digestion assays or techniques for determining digestibility or protein stability.

The article “a” and “an” are used herein to refer to one or more than one (i.e., to at least one) of the grammatical object of the article. By way of example, “an element” means one or more element.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

The present inventors have discovered a method of producing OXOX variants that have maintained or increased OXOX activity. As used interchangeably herein, “OXOX activity”, “biological activity of OXOX” or “functional activity of OXOX”, refers to an activity exerted by an OXOX enzyme as determined in vivo or in vitro, according to standard techniques.

In a preferred embodiment, OXOX activity is at least one or more of the following activities: (i) acting on oxalate, O₂, and H+ to produce CO₂ and H₂O₂ either in vitro or in vivo; or (ii) aiding a plant's resistance to a pathogen, for example, to a pathogen of Sclerotinia or (iii) any the activity of (i) or (ii).

In one embodiment, maintained OXOX activity or maintaining OXOX activity is at least one or more of the following activities: (i) acting on oxalate, O₂, and H+ to produce about the same level of CO₂ and H₂O₂ either in vitro or in vivo as compared to a native or wild type OXOX enzyme; (ii) maintaining Kₘ for oxalate and the like as compared to a native or wild type OXOX enzyme; (iii) maintaining k_{cat} of the following reaction of the OXOX acting on oxalate, O₂, and H+ to produce CO₂ and H₂O₂ as compared to a wild type OXOX enzyme; (iv) maintaining a plant's resistance to a pathogen relative resistance achieved by a wild type OXOX enzyme; (v) maintaining digestibility as compared to the digestibility of a wild type OXOX enzyme, e.g., a wild type fungal OXOX or a wheat OXOX enzyme ((Lane B G, et al. (1991) Homologies between Members of the Germin Gene Family in Hexaploid Wheat and Similarities between These Wheat Germins and Certain Physarum Spherulins. J. Biol. Chem. 266:10461-10469); (vi) maintaining digestibility as compared to the digestibility of a wild type OXOX enzyme when subjected to a SGF assay; (vii) maintaining an OXOX activity at a higher pH, e.g. a pH from about 3.5 to 6.0, as compared to the activity by a wild type OXOX enzyme at the same pH; (viii) maintaining an OXOX activity at an optimal pH, e.g. pH 3.5, as compared to OXOX activity a wild type OXOX enzyme at the same pH; (ix) maintaining a plant's resistance to an environmental stress such as heat, cold or drought, or mechanical damage or other abiotic stress relative to resistance achieved by a wild type OXOX enzyme; or (x) any of the activities of (i) to (ix).

In a preferred embodiment, increased OXOX activity or increasing OXOX activity is at least one or more of the following activities: (i) acting on oxalate, O₂, and H+ to produce an increased level of CO₂ and H₂O₂ either in vitro or in vivo; (ii) decreasing Kₘ for oxalate and the like as compared to a native or wild type OXOX enzyme; (iii) increasing kₐₜ of the following reaction of the OXOX acting on oxalate, O₂, and H+ to produce CO₂ and H₂O₂ as compared to the k_{cat} of a wild type OXOX enzyme; (iv) increasing a plant's resistance to a pathogen, for example, to a pathogen of Sclerotinia, as compared to resistance achieved by a wild type OXOX enzyme; (v) increasing digestibility of a known fungal OXOX or OXOX variant enzyme as compared to the digestibility of a wild type OXOX enzyme, e.g., a wild type fungal OXOX or a wheat OXOX enzyme ((Lane B G, et al. (1991) Homologies between Members of the Germin Gene Family in Hexaploid Wheat and Similarities between These Wheat Germins and Certain Physarum Spherulins. J. Biol. Chem. 266:10461-10469); (vi) increasing OXOX activity at a higher pH, e.g. a pH from about 3.5 to 6.0, as compared to OXOX activity of a wild type OXOX enzyme; (vii) increasing an OXOX activity at an optimal pH, e.g. pH 3.5, as compared to OXOX activity of a wild type OXOX enzyme at the same pH; (viii) increasing a plant's resistance to an environmental stress such as heat, cold or drought, or mechanical damage or other abiotic stress as compared to resistance achieved by a wild type OXOX enzyme; or (ix) any of the activities of (i) to (viii).

OXOX activity may be determined using any number of methods, including colorimetric assays that measure hydrogen peroxide levels (Laker et al, 1980), measuring specific activity (K_{cat}/K_{M}), synthetic gastric fluid (SGF) assay, and testing plants transformed with OXOX variants of the present invention for resistance to a pathogen, such as Sclerotinia (Hu, et al., Overexpression of a gene encoding hydrogen peroxide-generating oxalate oxidase evokes defense responses in sunflower. (2003) Plant Physiol. 133:170-181; Chen, Y., and Wang, D. Two convenient methods to evaluate soybean for resistance to Sclerotinia sclerotiorum. (2005) Plant Dis. 89:1268-1272) or combinations thereof.

In one aspect, the invention includes an isolated or recombinant polypeptide with increased OXOX activity relative to naturally occurring enzymes involved in oxalate degradation, e.g., a wild type OXOX enzyme. Generally, such polypeptides are OXOX's. For example, isolated or recombinant polypeptides of the invention have an OXOX activity that is at least about 1-fold, 1.5-fold, 2.0-fold, 2.5-fold, 3-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold, 11-fold, 11.5-fold, 12.0-fold, 12.5-fold, 13-fold, 13.5-fold, 14.0-fold, 14.5-fold, 15.0-fold, 15.5-fold, 16.0-fold, 16.5-fold, 17.0-fold, 17.5-fold, 18.0-fold, 18.5-fold, 19.0-fold, 19.5-fold, 20.0-fold, 21-fold, 21.5-fold, 22.0-fold, 22.5-fold, 23-fold, 23.5-fold, 24.0-fold, 24.5-fold, 25.0-fold, 25.5-fold, 26.0-fold, 26.5-fold, 27.0-fold, 27.5-fold, 28.0-fold, 28.5-fold, 29.0-fold, 29.5-fold, 30.0-fold, or greater than a naturally occurring (native or wild-type) enzyme, such as exemplified by any one of SEQ ID NOS:18 or 19 or codon-optimized enzyme such as exemplified by any one of SEQ ID NOS:21 or 22.

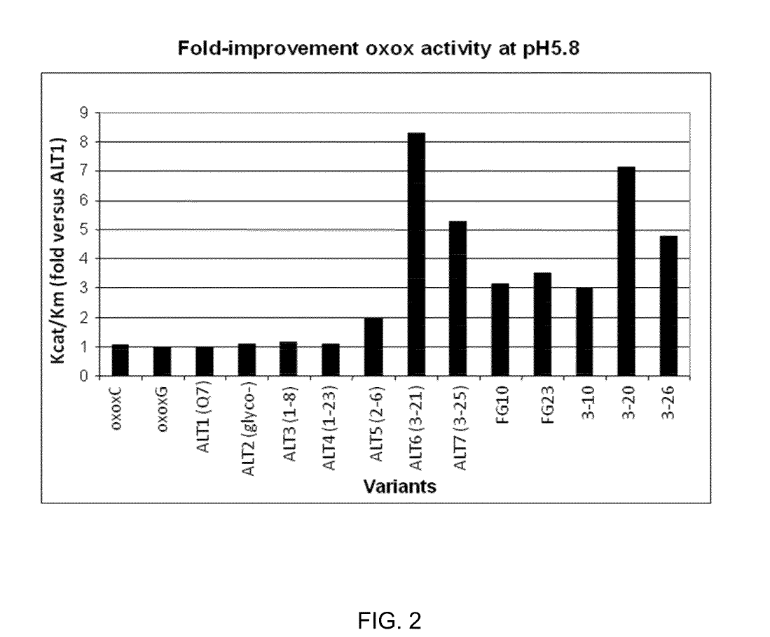

The polypeptides of the invention typically exhibit maintained or increased OXOX activity at a pH in the range of 3.5 to 6.0 than the activity exhibited by any of the naturally occurring OXOX enzymes, e.g., wild type OXOX, such as those represented by SEQ ID NOs:18 or 19 or codon-optimized OXOX enzyme such as, for example, SEQ ID NOs:21 or 22. For example, as shown in FIG. 2, the polypeptides of the invention exhibit an increased OXOX activity at a pH range of between about 3.5 and about 6.0. Frequently, the polypeptides of the invention exhibit the increased OXOX activity between about pH 3.5 and 6.0. Often, the increased OXOX activity is exhibited at a pH from about 3.8 to about 5.8. Polypeptides exhibiting a maintained or increased OXOX activity at about pH 5.8 are particularly useful for in vivo applications where detoxification occurs close to plant physiological pH level. See Table 5 described elsewhere herein.

For example, maintained or increased OXOX activity of an OXOX variant polypeptide can be conferred by alterations in the binding of, or alterations in the conversion activity of, an OXOX substrate such as oxalate. For example, the polypeptide of the invention having an increased OXOX activity can have a higher k_{cat} than any of the naturally occurring enzymes, e.g., exemplified by or achieved by any one of SEQ ID NOs:18 or 19. Alternatively, or in addition, the polypeptide of the invention may have a lower or decreased K_{M} than any of the naturally occurring enzymes or codon-optimized wild type enzymes described elsewhere herein, e.g. SEQ ID NOs:18 or 19 or an OXOX wild type enzyme codon-optimized for a plant such as SEQ ID NOs:18, 19, 21 or 22. Additionally, an OXOX variant polypeptide of the invention may increase a plant's resistance to a pathogen. As used herein, the term “pathogen” includes fungi, bacteria, nematodes, viruses, parasitic weeds, pests, biological agents, disease-producing microorganisms, toxic biological products, and organic biocides that can cause death or injury to plants.

In one aspect, the pathogen may be an oxalate-secreting pathogen. In another aspect, an OXOX variant polypeptide of the invention or known fungal OXOX polypeptide may maintain or increase digestibility of the OXOX variant protein or fungal OXOX protein as compared to the digestibility of a wild type OXOX, such as a plant OXOX, or a parental fungal OXOX, for example, when the protein is subjected to a protease or enzyme such as pepsin. Gel analysis of OXOX variants of 3F3, FG15, 4-85 and 3-25 subjected to a Simulated Gastric Fluid (SGF) digestibility assay shows that each variant is digested or degraded more rapidly than was the fungal OXOX-C.

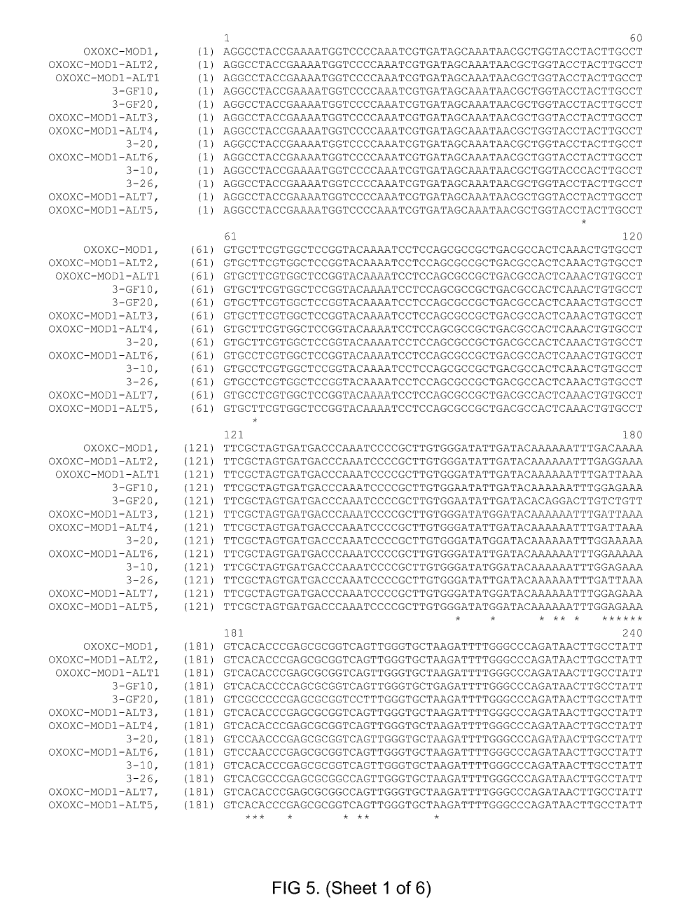

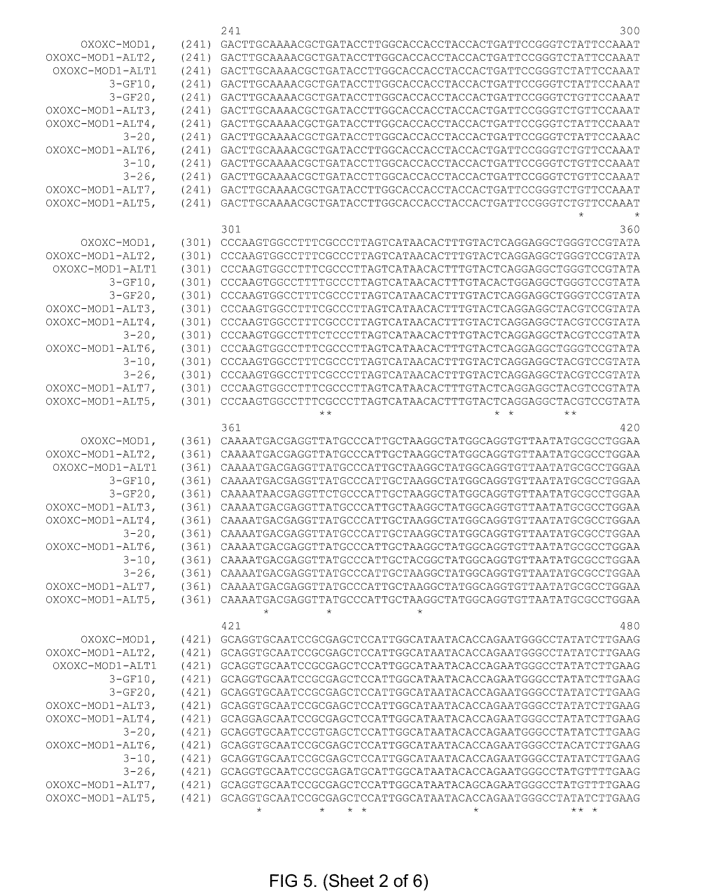

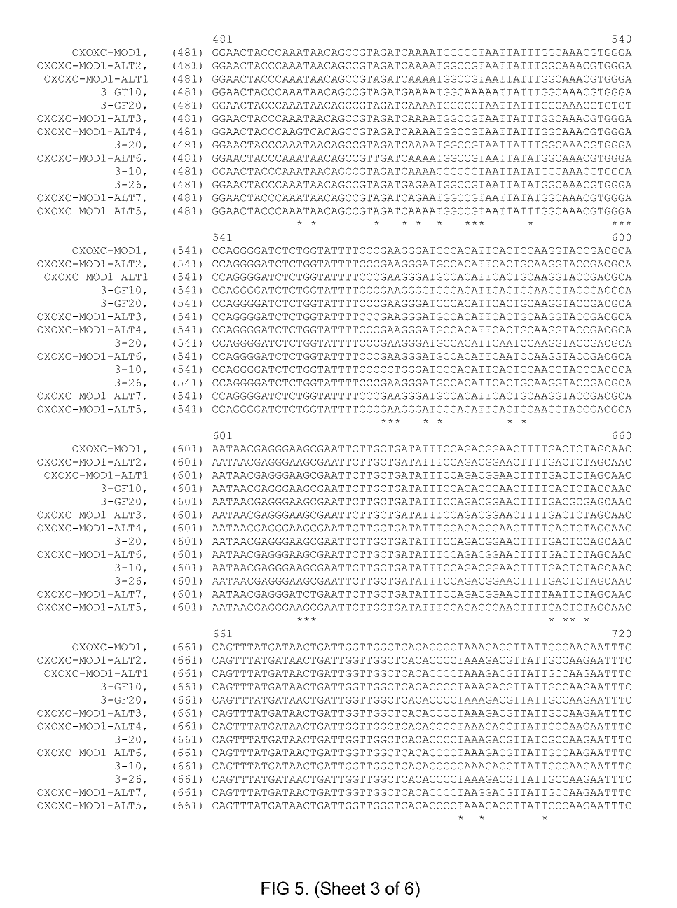

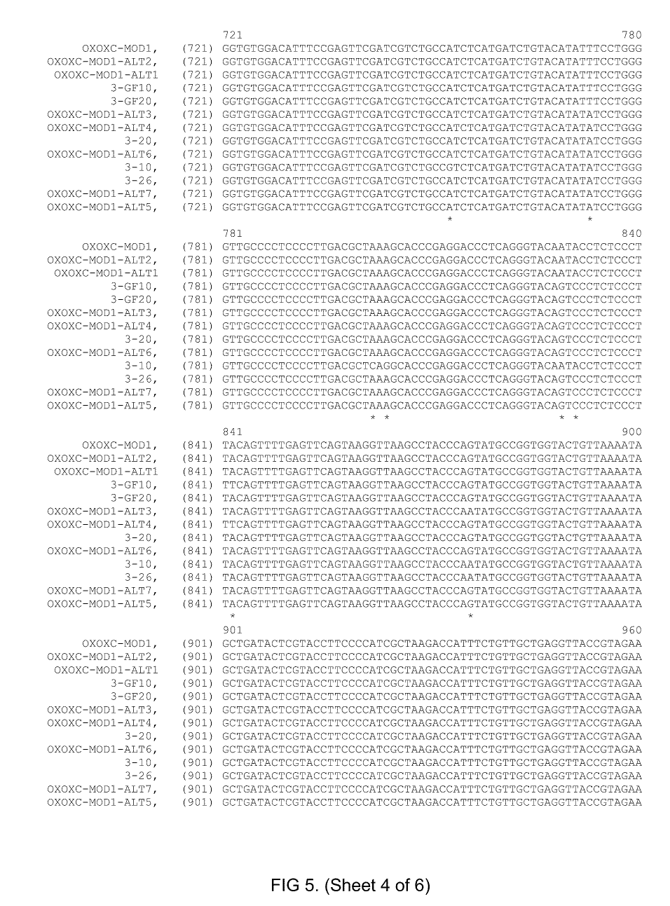

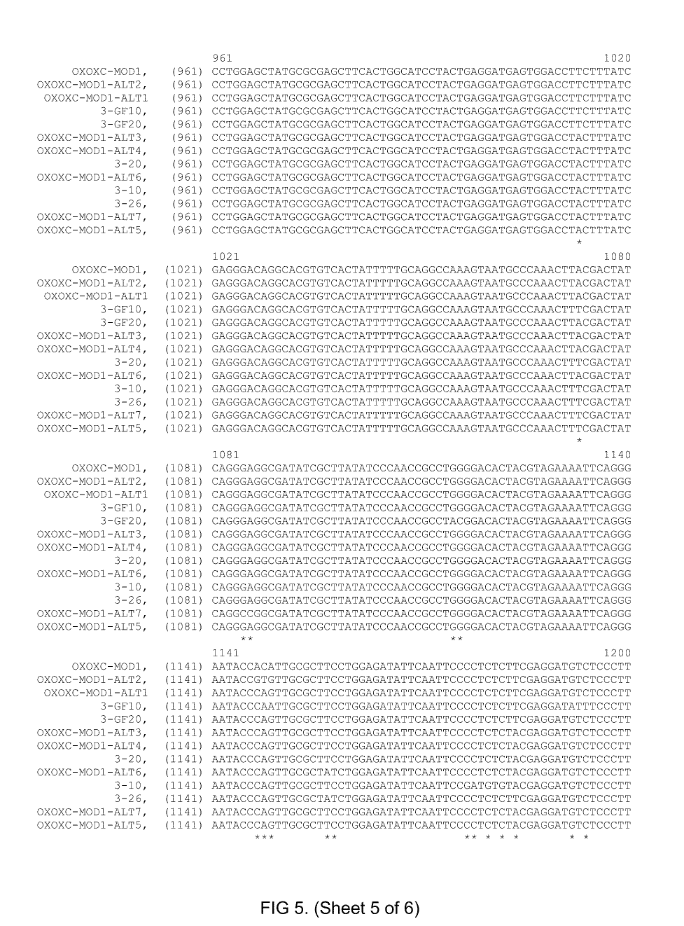

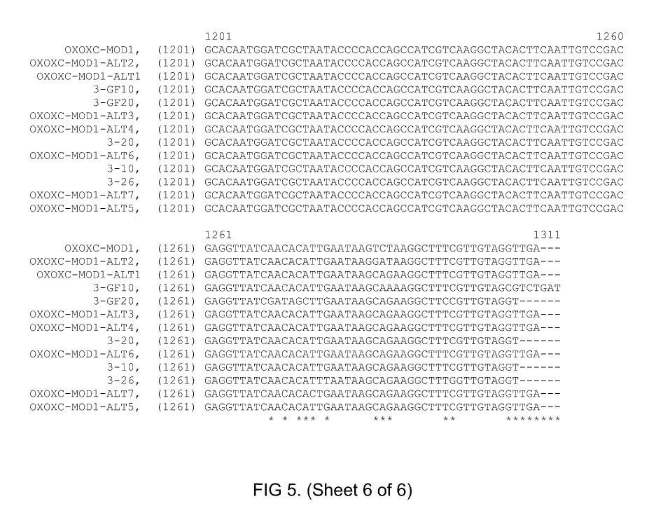

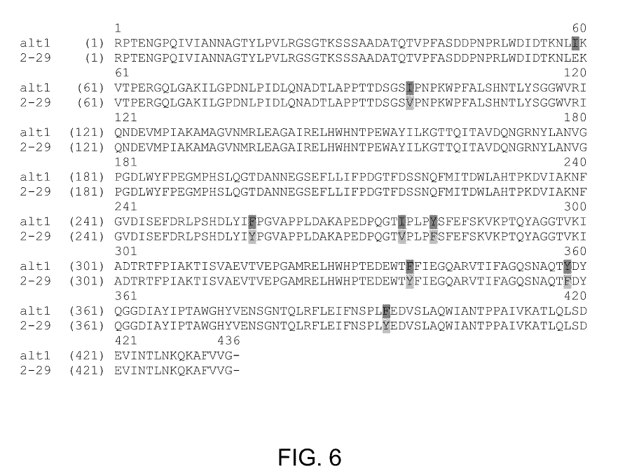

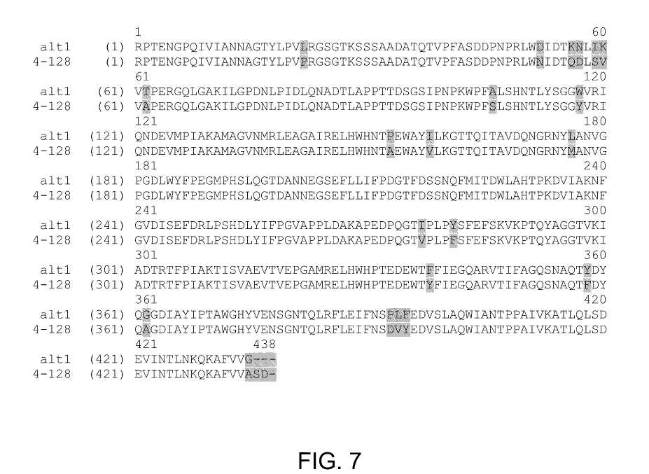

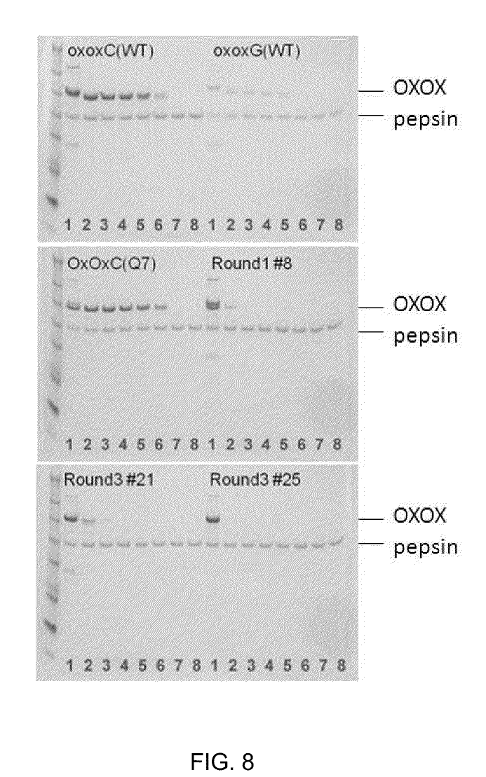

Compositions include plants having altered levels of OXOX and/or OXOX activities, including variant OXOX's of the present invention or known fungal OXOX's. Further provided are plants having an altered level of OXOX polypeptides or an active variant or fragment thereof and/or maintained or increased OXOX activity. Included are known isolated polynucleotides encoding fungal OXOX polypeptides having OXOX activity and isolated OXOX variant polynucleotides encoding OXOX variant polypeptides of the present invention. Also included are known fungal OXOX's and OXOX variants having maintained or increased OXOX activity than the OXOX activity of the wild-type OXOX, non-codon-optimized OXOX, codon-optimized OXOX, or non-fungal, plant OXOX. In one aspect, the activity is digestibility. In one aspect, the activity of the known fungal OXOX or variant OXOX is maintained or increased at a pH from about 3.5 to 6.0, than the activity exhibited by a naturally occurring OXOX enzyme (wild type OXOX) or plant OXOX. Additionally, the known fungal OXOX or OXOX variant polypeptide may have increased activity at a pH that is higher than an OXOX's typical optimal pH, e.g. a pH of 3.5. Accordingly, the known fungal OXOX or OXOX variant polypeptide may have increased activity at a pH from about 3.8 to about 5.8. In one aspect, the plants comprise the known fungal OXOX, OXOX variant polypeptide encoded by a polynucleotide having one or more of the substitutions shown in FIG. 5 or identified using any of the methods of the present invention.

In specific compositions, the plants have an altered level of OXOX, for example, for a known fungal OXOX, a variant OXOX of the present invention, or an OXOX identified by the methods of the invention or an active variant or fragment thereof. Any suitable fungal OXOX may be used in the methods and compositions described herein. These, include, but are not limited to known OXOX disclosed in published literature and public databases such as National Center for Biotechnology Information (NCBI) and the like. Exemplary OXOX's obtained from such sources are described elsewhere herein. Known fungal OXOX or variant OXOX for use in the methods and compositions include but are not limited to those from an oxalate producing fungus, an OXOX obtained from the genus of Sclerotinia, an OXOX obtained from the genus of Ceriporiopsis, an OXOX obtained from Ceriporiopsis subvermispora and the like. In some examples, the plants have an altered level and/or activity of an known fungal OXOX polypeptide or OXOX variant polypeptide having the amino acid sequence set forth in Genbank Accession No. Q5ZPV6|oxalate, Q5ZH54|oxalate, Q5ZH56|oxalate, Q5ZH55|oxalate, P26759|oxalate, P45850|oxalate, P15290|oxalate, P45851|oxalate, Q9FEW6|oxalate, Q8L695|oxalate, Q57TY7|oxalate, Q57XH4|oxalate, Q4DWT0|oxalate, and Q4CQK0|oxalate. With respect to OXDC sequences, over 26 different OXDC homologues are available in Genbank alone, including but not limited to Q81GZ6|Oxalate, Q3EPK1|Oxalate, O34767|Oxalate, Q5WJS8|Oxalate, Q3EK26|Oxalate, Q3EK27|Oxalate, O34714|Oxalate, A2QFX7|Oxalate, Q9UVK4|Oxalate, A0TTC9|Oxalate, A0B0Z6|Oxalate, Q1BNY3|Oxalate, Q4BLX2|Oxalate, Q397J1|Oxalate, A0T5N3|Oxalate, Q0B8F6|Oxalate, A1UV39|Oxalate, Q3JFC8|Oxalate, A2RXA0|Oxalate, Q2J3P9|Oxalate, Q02AS8|Oxalate, A0IL94|Oxalate, Q31KK1|Oxalate, Q81DI3|Oxalate, Q3ELX3|Oxalate, or A0QUL8|Oxalate.

These, include, but are not limited to OXOX variant polypeptides having one or more of the amino acid substitutions listed in Tables 2 and 3. For example, the OXOX variant polypeptide comprises an amino acid sequence that has been substituted with at least one amino acid substitution at a position that that corresponds to position 10, 19, 23, 26, 29, 35, 36, 38, 39, 40, 53, 54, 57, 58, 60, 61, 62, 63, 65, 68, 72, 79, 81, 83, 99, 102, 107, 115, 118, 124, 127, 131, 144, 148, 154, 159, 164, 166, 171, 174, 177, 181, 190, 192, 196, 200, 202, 203, 218, 219, 245, 259, 269, 278, 282, 287, 289, 290, 339, 349, 353, 359, 363, 373, 384, 387, 394, 395, 396, 399, 410, 425, 426, 427, 430, 433 or 436 of the amino acid sequence of the OXOX polypeptide of SEQ ID NO:37 or additional one or two amino acid residues at position 437 or 438 of the amino acid sequence of the OXOX polypeptide of SEQ ID NO:37 or substitutions that are a combination thereof. In some examples, the plants have an altered level and/or activity of an OXOX variant polypeptide having the amino acid sequence set forth in SEQ ID NO: 18, 19, 21, 22, 23, 24, 25, 26, 27, 28, 29, 30, 31, 32, 33, 34, 66, 67, 68, 69, 70, 71, 72, 73, 74, 75, 76, 77, 78, 79, 80, 81, 82, 83, 84, 85, 86, 87, 88, 89, 90, 91, 92, 100, 101, 102, 103, 104, 105, or 106 or an active variant or fragment thereof. Further provided are plants having an altered level and/or activity of the OXOX polypeptide encoded by a polynucleotide set forth in SEQ ID NO: 1, 2, 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 58, 59, 60, 61, 62, 63, 64, 65, 93, 94, 95, 96, 97, 98, or 99 or an active variant or fragment thereof. The variant may have maintained or increased OXOX activity compared to the wild type or native OXOX, for example, of increased disease resistance to a pathogen, for example, an oxalate-secreting pathogen. In some cases, the activity may be increased relative to a wild type OXOX activity as pH becomes higher, e.g. from a pH from about 3.5 to about 6.0, preferably from a pH range of about 3.8 to about 5.8. The plants of the invention or part thereof may exhibit modulation in digestibility.

In another aspect, the plants have an altered level of OXOX activity when, a known fungal OXOX polypeptide, an OXOX variant polypeptide of the present invention or identified by the methods of the present invention or an active variant or fragment thereof is expressed in a plant cell. The variants can be tested to determine OXOX activity as described elsewhere herein.

In one embodiment, a method of the present invention includes identifying OXOX variants with at least one maintained or increased OXOX activity. Preferably the variants are obtained from a fungal OXOX. The method includes modifying OXOX polynucleotides to generate an OXOX variant polynucleotide that encodes a polypeptide that has at least one OXOX activity as described elsewhere herein.

In one aspect, the method involves identifying amino acid substitutions in OXOX's that confer a functional OXOX enzyme with maintained or increased OXOX activity. The OXOX variants may be generated using shuffling or site-directed mutagenesis or other methods known to one skilled in the art. The variants, including OXOX polypeptides or polynucleotides, may be assayed for OXOX activity in vitro or in vivo.

Glycosylation sites may be present in a native or wild type OXOX polypeptide. Without wishing to be bound by this theory, it is believed that since fungal OXOX have no significant homology to allergens and by eliminating glycosylation sites in OXOX polypeptides, the employed OXOX variants or fungal OXOX advantageously will remove or decrease any potential allergenicity to an animal when consumed and maintain or increase digestibility of the fungal OXOX or OXOX variant polypeptide. In some examples, OXOX activity will be maintained or increased as compared to a control. In contrast, the OXOX protein from wheat, for example, is very stable protein that cannot be quickly degraded by simulated human gastric fluid, posing potential allergenicity risks if the protein is used in a food product. Furthermore, OXOX proteins from plants may also have potential allergenicity risks since many plant OXOX's have sequence homologies to seed storage proteins and to a black pepper spice allergen. Accordingly, the method includes modifying an OXOX polynucleotide where at least one potential glycosylation site in the encoded OXOX variant polypeptide is eliminated. In some cases, two, three or more glycosylation sites are eliminated. In some cases, all glycosylation sites are eliminated. In one aspect, one of the glycosylation sites at position 60, 384, 430 of SEQ ID NO:37 has been eliminated by an amino acid substitution at that position. In particular, the amino acid residues of T, T and S at positions of 60, 384, 430 of SEQ ID NOS:26 or 57 may be substituted with I, Q and Q or I, V and D, respectively. In one aspect, shuffled gene variants can be screened for OXOX activity in E. coli via enzyme activity assays, such as by oxalate oxidase assays. The variants with OXOX activity or increased OXOX activity can then be used to transform a plant for resistance to a pathogen, such as Sclerotinia. The host cell including an OXOX variant may be assayed to identify one or more mutations (substitutions) or polymorphisms in the OXOX, for example, a mutation or polymorphism that modifies OXOX activity, for example, increases OXOX activity. In other aspects, the method includes employing a fungal OXOX polynucleotide encoding an OXOX polypeptide. In some examples, the fungal OXOX polynucleotide encodes an OXOX variant polypeptide.

In addition, the present invention provides novel compositions and methods for modulating, for example, increasing or decreasing, the level of OXOX protein in a plant cell or plant. In particular, the polynucleotides and polypeptides of the present invention can be used to generate transgenic plants expressing known fungal OXOX's or variant OXOX's of the present invention. Described herein are at least 44 novel OXOX variants and at least 68 substitutions that alone or in combination may maintain or increase OXOX activity, including resistance to Sclerotinia. Modulation of the OXOX's of the present invention would provide a mechanism for increasing a plant's resistance to a pathogen, for example, to an oxalate-secreting pathogens such as Sclerotinia. Thus, one embodiment provides methods for modulating, for example, increasing or decreasing, a plant's resistance to a pathogen using known fungal OXOX polynucleotides and polypeptides, OXOX variant polynucleotides and polypeptides of the present invention, or OXOX variants identified by methods of the present invention.

Variants of OXOX polynucleotides of the present invention encoding OXOX variants having amino acid substitutions and maintained or increased OXOX activity may be created by any number of methods, including but not limited to shuffling, site-directed mutagenesis, and the like. For example, routine molecular biology techniques may be used to substitute 1, 2, 3, 4, 5, 6, 7, 8, 9, 10, 11, 12, 13, 14, 15, 16, 17, 18, 19, 20, 25, 30, 35, 40, 45, 50, 55 or more amino acid residues in an OXOX polypeptide so that the substituted OXOX polypeptide differs from the polypeptide encoded by the parental or template OXOX polynucleotide. In one aspect, the parental or template polynucleotide is endogenous to a fungal organism. In one aspect, the parental or template polynucleotide is a wild type fungal OXOX, for example, OXOX polynucleotides from a fungi, such as the dikaryotic white rot fungus Ceriporiopsis subvermispora. See Escutia et al. (Cloning and sequencing of two Ceriporiopsis subvermispora bicupin oxalate oxidase allelic isoforms: implications for the reaction specificity of oxalate oxidases and decarboxylases. (2005)). Additionally, putative homologs of the Ceriporiopsis subvermispora sequences of Escutia et al. may be identified and isolated from other microbes and used in gene shuffling to increase sequence diversity.

In one aspect, the parental or template polynucleotide is a fungal oxalate decarboxylase (OXDC), for example, OXDC polynucleotides from a fungi, such as a wild type OXDC. (Burrell et al, Oxalate decarboxylase and oxalate oxidase activities can be interchanged with a specificity switch of up to 282,000 by mutating an active site lid. (2007) Biochemistry, 46, 12327-12336.) As used herein, the term “oxalate decarboxylase” or “OXDC” refers to an enzyme that acts on oxalate and H⁺ to produce formate and CO₂. The OXOX and/or OXDC polynucleotide may be codon-optimized for a particular plant, e.g. soybean or maize, prior to or subsequent to shuffling.

### Shuffling

OXOX variant polynucleotides may be generated by any suitable shuffling method, for example, from one or more parental OXOX or OXDC sequences or a combination thereof. The shuffling may optionally include mutagenesis, in vitro manipulation, in vivo manipulation of one or more sequences or in silico manipulation of sequences. The resultant shuffled polynucleotides may be introduced into a suitable host cell, typically in the form of expression cassettes wherein the shuffled polynucleotide sequence encoding the OXOX may be operably linked to a transcriptional regulatory sequence and any necessary sequences for ensuring transcription, translation, and processing of the encoded OXOX protein.

Each such expression cassette or its shuffled OXOX encoding sequence can be referred to as a “library member” composing a library of shuffled OXOX sequences. In one aspect, E. coli libraries may be constructed from single gene shuffling or semi-synthetic shuffling or combinations thereof in which the oligonucleotides are “spiked” to contain amino acid substitutions that differ from wild type OXOX's endogenous to a plant cell. See Examples 5 as described herein. The library may be introduced into a population of host cells, such that individual host cells receive substantially one or a few species of library member(s), to form a population of shufflant host cells expressing a library of shuffled OXOX species.

A variety of OXOX and OXDC genomic, cDNA, mRNA sources are known and can be used in the recombination processes herein. Coding sequences for OXOX for various species are disclosed in the literature and Genbank, among other public sources, and may be obtained by cloning, PCR, or from deposited materials. For example, as noted, a variety of references herein describe such genes. For example, Escutia et al. (Cloning and sequencing of two Ceriporiopsis subvermispora bicupin oxalate oxidase allelic isoforms: implications for the reaction specificity of oxalate oxidases and decarboxylases. (2005)) describes several OXOX genes as do other publications, for example, oxalate oxidase from barley (marketed by Boehringer, ref. 567698), from sorghum (Pundier, Phytochemistry, (1991), 30, 4, 1065) or from the moss Mnium menziesii (Laker et al. Spectrophotometric determination of urinary oxalate with oxalate oxidase prepared from moss. (1980). Clin Chem 26:827-830).

A protein with oxalate oxidase activity which is particularly appreciated is wheat germin, whose sequence has been described by Dratewka-Kos, J. Biol. Chem., (1989), 264, 4896) and Lane et al. ((1991) Homologies between Members of the Germin Gene Family in Hexaploid Wheat and Similarities between These Wheat Germins and Certain Physarum Spherulins. J. Biol. Chem. 266:10461-10469). Taking into account the degeneracy of the genetic code, a large number of nucleotide sequences encoding oxalate oxidase exist which can also be used for the purposes of the invention. Examples of public databases that include OXOX and OXDC sources include: Genbank: ncbi.nlm.nih.gov/genbank/: EMBL: ebi.ac.uk.embl/: as well as, e.g., the protein databank, Brookhaven Laboratories; the University of Wisconsin Biotechnology Center, the DNA databank of Japan, Laboratory of genetic Information Research, Misuina, Shizuda, Japan. As noted, over 14 different OXOX homologues are available in Genbank alone, for example, Q5ZPV6|oxalate, Q5ZH54|oxalate, Q5ZH56|oxalate, Q5ZH55|oxalate, P26759|oxalate, P45850|oxalate, P15290|oxalate, P45851|oxalate, Q9FEW6|oxalate, Q8L695|oxalate, Q57TY7|oxalate, Q57XH4|oxalate, Q4DWT0|oxalate, and Q4CQK0|oxalate. With respect to OXDC sequences, over 26 different OXDC homologues are available in Genbank alone, including but not limited to Q81GZ6|Oxalate, Q3EPK1|Oxalate, O34767|Oxalate, Q5WJS8|Oxalate, Q3EK26|Oxalate, Q3EK27|Oxalate, O34714|Oxalate, A2QFX7|Oxalate, Q9UVK4|Oxalate, A0TTC9|Oxalate, A0B0Z6|Oxalate, Q1BNY3|Oxalate, Q4BLX2|Oxalate, Q397J1|Oxalate, A0T5N3|Oxalate, Q0B8F6|Oxalate, A1UV39|Oxalate, Q3JFC8|Oxalate, A2RXA0|Oxalate, Q2J3P9|Oxalate, Q02AS8|Oxalate, A0IL94|Oxalate, Q31KK1|Oxalate, Q81DI3|Oxalate, Q3ELX3|Oxalate, and A0QUL8|Oxalate.

The following publications describe a variety of recursive recombination procedures and/or methods which can be incorporated into such procedures, e.g., for shuffling of OXOX and/or OXDC polynucleotides and/or fragments: Stemmer, et al., (1999) “Molecular breeding of viruses for targeting and other clinical properties. Tumor Targeting” 4:1-4; Nesset al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameri et al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proceedings of the National Academy of Sciences, U.S.A. 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling”, Nature Biotechnology 14:315-319; Gates et al. (1996) “Affinity selective isolation of ligands from peptide libraries through display on a lac repressor ‘headpiece dimer’” Journal of Molecular Biology 255:3732 386; Stemmer (1996) “Sexual PCR and Assembly PCR” In: The Encyclopedia of Molecular Biology. VCH Publishers, New York. pp. 447-457; Crameri and Stemmer (1995) “Combinatorial multiple cassette mutagenesis creates all the permutations of mutant and wildtype cassettes” BioTechniques 18:194-195; Stemmer et al., (1995) “Single-step assembly of a gene and entire plasmid form large numbers of oligodeoxyribonucleotides” Gene, 164:49-53; Stemmer (1995) “The Evolution of Molecular Computation” Science 270:1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: in vitro recombination for molecular evolution.” Proceedings of the National Academy of Sciences. U.S.A. 91:10747-10751.

In addition, details and formats for DNA shuffling are found in a variety of PCT and foreign patent application publications, including: Stemmer and Crameri, “DNA Mutagenesis by Random Fragmentation and Reassembly” WO95/22625; Stemmer and Lipschutz “End Complementary Polymerase Chain Reaction” WO96/33207; Stemmer and Crameri “Methods for Generating Polynucleotides Having Desired Characteristics by Iterative Selection and Recombination” WO97/0078; Minshul and Stemmer, “Methods and Compositions for Cellular and Metabolic Engineering” WO97/35966; Punnonen et al. “Targeting of Genetic Vaccine Vectors” WO99/41402; Punnonen et al. “Antigen Library Immunization” WO99/41383; Punnonen et al. “Genetic Vaccine Vector Engineering” WO99/41369; Punnonen et al. “Optimization of Immunomodulatory Properties of Genetic Vaccines” WO99/41368; Stemmer and Crameri, “DNA Mutagenesis by Random Fragmentation and Reassembly” EP 0934999; Stemmer “Evolving Cellular DNA Uptake by Recursive Sequence Recombination” EP 0932670; Stemmer et al., “Modification of Virus Tropism and Host Range by Viral Genome Shuffling” WO99/23107; Apt et al., “Human Papillomavirus Vectors” WO99/21979; Del Cardayre et al. “Evolution of Whole Cells and Organisms by Recursive Sequence Recombination” WO98/31837; Patten and Stemmer, “Methods and Compositions for Polypeptide Engineering” WO98/27230; Stemmer et al., and “Methods for Optimization of Gene Therapy by Recursive Sequence Shuffling and Selection” WO98/13487.

As review of the foregoing publications, patents, published applications and U.S. patent applications reveals, recursive recombination and selection of polynucleotides to provide new OXOX variant polynucleotides with maintained or increased OXOX activity can be carried out by a number of established methods. Any of these methods can be adapted to the present invention to evolve OXOX coding polynucleotides or homologues to produce new OXOX variant polypeptides with maintained or increased OXOX activity. Both the methods of making such enzymes and the enzymes or enzyme coding libraries produced by these methods are encompassed by the present invention.

A number of different general classes of recombination methods may be used to generate OXOX's of the present invention. First, polynucleotides can be recombined in vitro by any of a variety of techniques discussed in the references above, including e.g., DNAse digestion of polynucleotides to be recombined followed by ligation and/or PCR reassembly of the polynucleotides. Second, polynucleotides can be recursively recombined in vivo, e.g., by allowing recombination to occur between polynucleotides in cells. Third, whole cell genome recombination methods can be used in which whole genomes of cells are recombined, optionally including spiking of the genomic or recombination mixtures so that they encode the desired amino acid substitutions shown to produce functional OXOX enzymes. See Example 5. Fourth, synthetic recombination methods can be used, in which oligonucleotides corresponding to different OXOX homologues are synthesized and reassembled in PCR or ligation reactions which include oligonucleotides which correspond to more than one parental polynucleotide, thereby generating new recombined polynucleotides. Oligonucleotides can be made by standard nucleotide addition methods, or can be made, e.g., by tri-nucleotide synthetic approaches. Fifth, in silico methods of recombination can be affected in which genetic algorithms are used in a computer to recombine sequence strings which correspond to OXOX homologues. The resulting recombined sequence strings are optionally converted into polynucleotides by synthesis of polynucleotides which correspond to the recombined sequences, e.g., in concert with oligonucleotide synthesis/gene reassembly techniques. Any of the preceding general recombination formats can be practiced in a reiterative fashion to generate a more diverse set of recombinant polynucleotides.

Combinations of in vitro and in vivo shuffling may be used to enhance combinatorial diversity. As mentioned previously, “in silico” shuffling may be used to generate OXOX variant polynucleotides using computer algorithms to perform “virtual” shuffling using genetic operators in a computer. In silico shuffling may be described in detail in Selifonov and Stemmer in “Methods for Making Character Strings, Polynucleotides & Polypeptides Having Desired Characteristics” filed Feb. 5, 1999, U.S. Ser. No. 60/118,854 and “Methods for Making Character Strings, Polynucleotides & Polypeptides Having Desired Characteristics” by Selifonov et al. filed Oct. 12, 1999 (U.S. Ser. No. 09/416,375).

One advantage of oligonucleotide-mediated recombination may be the ability to recombine homologous polynucleotides with low sequence similarity, or even non-homologous polynucleotides. In these low-homology oligonucleotide shuffling methods, one or more set of fragmented polynucleotides (e.g., oligonucleotides corresponding to multiple OXOX polynucleotides) are recombined, e.g., with a set of crossover family diversity oligonucleotides. Each of these crossover oligonucleotides have a plurality of sequence diversity domains corresponding to a plurality of sequence diversity domains from homologous or non-homologous polynucleotides with low sequence similarity. The fragmented oligonucleotides, which are derived by comparison to one or more homologous or non-homologous polynucleotides, can hybridize to one or more region of the crossover oligonucleotides, facilitating recombination.

When recombining homologous polynucleotides, sets of overlapping family gene shuffling oligonucleotides (which are derived by comparison of homologous polynucleotides, by synthesis of corresponding oligonucleotides) are hybridized and elongated (e.g., by reassembly PCR or ligation), providing a population of recombined polynucleotides, which can be selected for a desired trait or property. The set of overlapping family shuffling gene oligonucleotides includes a plurality of oligonucleotide member types which have consensus region subsequences derived from a plurality of homologous target polynucleotides.

In one aspect, family gene shuffling oligonucleotides that include one or more OXOX polynucleotide(s) are provided by aligning homologous polynucleotide sequences to select conserved regions of sequence identity and regions of sequence diversity. A plurality of family gene shuffling oligonucleotides may be synthesized (serially or in parallel) which correspond to at least one region of sequence diversity.

Sets of fragments, or subsets of fragments used in oligonucleotide shuffling approaches can be provided by cleaving one or more homologous polynucleotides (e.g., with a DNase), or, more commonly, by synthesizing a set of oligonucleotides corresponding to a plurality of regions of at least one polynucleotide (typically oligonucleotides corresponding to a full-length polynucleotide may be provided as members of a set of polynucleotide fragments). Cleavage fragments may be used in conjunction with family gene shuffling oligonucleotides, e.g., in one or more recombination reaction to produce recombinant OXOX polynucleotide(s).

Another approach of shuffling may be found in “Shuffling of Codon Altered Genes” by Patten et al. filed Sep. 29, 1998, (U.S. Ser. No. 60/102,362), Jan. 29, 1999 (U.S. Ser. No. 60/117,729), and Sep. 28, 1999, PCT/US99/22588. One way of generating diversity in a set of polynucleotides to be shuffled (i.e., as applied to the present invention, OXOX polynucleotides), may be to provide “spiked” polynucleotides containing mutations to eliminate glycosylation sites, decrease K_{M}, increase K_{cat} by synthesizing polynucleotides in which the nucleotides which encode certain amino acid residues are altered, it may be possible to access a completely different mutational spectrum upon subsequent mutation of the polynucleotide. This increases the sequence diversity of the starting polynucleotides for shuffling protocols, which alters the rate and results of forced evolution procedures. Codon modification procedures can be used to modify any OXOX polynucleotide or shuffled polynucleotide, e.g., prior to performing DNA shuffling.

The above references provide these and other basic recombination formats as well as many modifications of these formats. Regardless of the format which may be used, the polynucleotides of the invention can be recombined (with each other or with related or even unrelated) polynucleotides to produce a diverse set of recombinant polynucleotides, including homologous polynucleotides.

Thus, in a general aspect, a sequence shuffling method provides for generating libraries or cells containing recombinant OXOX polynucleotides that may be screened for OXOX activity, for example, increased OXOX activity. Libraries of recombinant polynucleotides are generated from a population of related-sequence polynucleotides which comprise sequence regions which have substantial sequence identity and can be homologously recombined in vitro or in vivo. In the method, at least two species of the related-sequence polynucleotides are combined in a recombination system suitable for generating sequence-recombined polynucleotides, wherein said sequence-recombined polynucleotides comprise a portion of at least one first species of a related-sequence polynucleotide with at least one adjacent portion of at least one second species of a related-sequence polynucleotide. Recombination systems suitable for generating sequence-recombined polynucleotides can be either: (1) in vitro systems for homologous recombination or sequence shuffling via amplification or other formats or (2) in vivo systems for homologous recombination or site-specific recombination.

The population of sequence-recombined OXOX polynucleotides comprises a subpopulation of polynucleotides which are suspected of encoding polypeptides with OXOX activity, preferably increased OXOX activity. The selected sequence-recombined polynucleotides may be subjected to at least one recursive cycle wherein at least one selected sequence-recombined polynucleotide may be combined with at least one distinct species of related-sequence polynucleotide (which may itself be a selected sequence-recombined polynucleotide) in a recombination system suitable for generating sequence-recombined polynucleotides, such that additional generations of sequence-recombined polynucleotide sequences are generated from the selected sequence-recombined polynucleotides obtained by the selection or screening method employed. In this manner, recursive sequence recombination generates library members which are sequence-recombined OXOX and/or OXDC polynucleotides possessing increased OXOX activity.

Polynucleotide sequence shuffling may be a method for recursive in vitro or in vivo homologous or non-homologous recombination of pools of OXOX and/or OXDC polynucleotide fragments or polynucleotides (e.g., genes from fungal organisms or portions thereof). Mixtures of related OXOX and/or OXDC polynucleotide sequences or polynucleotides are randomly or pseudorandomly fragmented, and reassembled to yield a library or mixed population of recombinant polynucleotides or polypeptides having OXOX activity. In an embodiment, the polynucleotides are fungal OXOX and/or OXDC polynucleotides or combinations thereof.

The present invention may be directed to a method for generating a selected OXOX polynucleotide sequences or population of selected polynucleotide sequences, typically in the form of amplified and/or cloned polynucleotides, whereby the selected polynucleotide sequence(s) encode an OXOX variant polypeptide that can be selected for, and whereby the selected polypeptide sequences have OXOX activity, for example, maintained or increased OXOX activity. In a preferred embodiment, the generated polynucleotides lack one or more glycosylation sites, for example, glycosylation sites found in a native (wild type) or parental template used in shuffling.

In a general aspect, the invention provides a method for generating libraries of recombinant polynucleotides having a subpopulation of library members which encode an OXOX variant protein having maintained or increased OXOX activity. Libraries of recombinant polynucleotides may be generated from a population of related-sequence OXOX and/or OXDC polynucleotides which comprise sequence regions which have substantial sequence identity and can be homologously recombined in vitro or in vivo. In another aspect, the libraries may be “spiked” to contain mutations not found in wild type plant OXOX's and that are found to produce functional OXOX enzymes and/or increased OXOX activity.

In one aspect, OXOX and OXDC polynucleotides are combined in a recombination system suitable for generating sequence-recombined polynucleotides. In one aspect, the method includes an OXOX endogenous to the host cell as a template, for example, a shuffling template. In one aspect, the template is an OXOX gene or cDNA or other nucleotide sequence from Ceriporiopsis or a species of Ceriporiopsis or other fungal gene. In a preferred embodiment, the polynucleotides are from the dikaryotic white rot fungus subvermispora. See Escutia et al. (Cloning and sequencing of two Ceriporiopsis subvermispora bicupin oxalate oxidase allelic isoforms: implications for the reaction specificity of oxalate oxidases and decarboxylases. (2005)). Additionally, putative homologs of the Ceriporiopsis subvermispora sequences of Escutia et al. identified and isolated from other microbes may be used in gene shuffling to increase sequence diversity. As mentioned herein, any number of OXDC sequences are known and may be used in the present invention, e.g. Genbank Q81GZ6|Oxalate, Q3EPK1|Oxalate, O34767|Oxalate, Q5WJS8|Oxalate, Q3EK26|Oxalate, Q3EK27|Oxalate, O34714|Oxalate, A2QFX7|Oxalate, Q9UVK4|Oxalate, A0TTC9|Oxalate, A0B0Z6|Oxalate, Q1BNY3|Oxalate, Q4BLX2|Oxalate, Q397J1|Oxalate, A0T5N3|Oxalate, Q0B8F6|Oxalate, A1UV39|Oxalate, Q3JFC8|Oxalate, A2RXA0|Oxalate, Q2J3P9|Oxalate, Q02AS8|Oxalate, A0IL94|Oxalate, Q31KK1|Oxalate, Q81DI3|Oxalate, Q3ELX3|Oxalate, and A0QUL8|Oxalate.

The polynucleotides may be from different organisms or species if desired. Recombination systems suitable for generating sequence-recombined polynucleotides can be either: (1) in vitro systems for homologous recombination or sequence shuffling via amplification or other formats described herein, or (2) in vivo systems for homologous recombination or site-specific recombination as described herein, or template-switching of a retroviral genome replication event. The population of sequence-recombined polynucleotides comprises a subpopulation of OXOX and/or OXDC polynucleotides which possess desired or advantageous enzymatic characteristics and which can be selected by a suitable selection or screening method. The selected sequence-recombined polynucleotides, which may be related-sequence OXOX and/or OXDC polynucleotides, can then be subjected to at least one recursive cycle wherein at least one selected sequence-recombined OXOX and/or OXDC polynucleotide may be combined with another related-sequence OXOX and/or OXDC polynucleotide (which may itself be a selected sequence-recombined polynucleotide) in a recombination system suitable for generating sequence-recombined polynucleotides with OXOX activity, such that additional generations of sequence-recombined polynucleotide sequences are generated from the selected sequence-recombined polynucleotides obtained by the selection or screening method employed. In this manner, recursive sequence recombination generates library members which are sequence-recombined polynucleotides possessing maintained or increased OXOX activity.

In one aspect, OXOX and/or OXDC polynucleotides, e.g. library members, may be fragmented and homologously recombined by PCR in vitro. Fragment generation may be by nuclease digestion, partial extension PCR amplification, PCR stuttering, or other suitable fragmenting means, such as described herein and in WO95/22625 published Aug. 24, 1995, and in commonly owned U.S. Ser. No. 08/621,859 filed Mar. 25, 1996, PCT/US96/05480 filed Apr. 18, 1996, which are incorporated herein by reference. Stuttering may be fragmentation by incomplete polymerase extension of templates. A recombination format based on very short PCR extension times can be employed to create partial PCR products, which continue to extend off a different template in the next (and subsequent) cycle(s), and effect de facto fragmentation. Template-switching and other formats which accomplish sequence shuffling between a plurality of OXOX sequence-related polynucleotides, including OXDC polynucleotides, can be used. Such alternative formats will be apparent to those skilled in the art.

In one aspect, OXOX and/or OXDC polynucleotides, e.g. library members, may be fragmented in vitro, the resultant fragments transferred into a host cell or organism and homologously recombined to form shuffled polynucleotides, in vivo. In one aspect, OXOX and/or OXDC polynucleotides, e.g. library members, may be cloned or amplified on episomally replicable vectors, a multiplicity of said vectors may be transferred into a cell and homologously recombined to form OXOX polynucleotides, e.g. library members, in vivo.

In one aspect, OXOX and/or OXDC polynucleotides, e.g. library members, may be not fragmented, but may be cloned or amplified on an episomally replicable vector as a direct repeat or indirect (or inverted) repeat, which each repeat comprising a distinct species of selected OXOX and/or OXDC polynucleotide sequences, said vector may be transferred into a cell and homologously recombined by intra-vector or inter-vector recombination to form shuffled library members in vivo.

In one aspect, combinations of in vitro and in vivo shuffling are provided to enhance combinatorial diversity. The recombination cycles (in vitro or in vivo) can be performed in any order desired by the practitioner. In one aspect, the first plurality of selected library members may be fragmented and homologously recombined by PCR in vitro. Fragment generation may be by nuclease digestion, partial extension PCR amplification, PCR stuttering, or other suitable fragmenting means, such as described herein and in the documents incorporated herein by reference. Stuttering may be fragmentation by incomplete polymerase extension of templates.

In one aspect, OXOX and/or OXDC polynucleotides, e.g. library members, may be fragmented in vitro, the resultant fragments transferred into a host cell or organism and homologously recombined to form shuffled polynucleotides, e.g. library members, in vivo. In an aspect, the host cell may be a unicellular photosynthetic eukaryotic organism or a plant cell. In one aspect, the plant cell has been engineered to contain enhanced recombination systems, such as an enhanced system for general homologous recombination (e.g., a plant expressing a recA protein or a plant recombinase from a transgene or plant virus) or a site-specific recombination system (e.g., a cre/LOX or frt/FLP system encoded on a transgene or plant virus).

In one aspect, OXOX and/or OXDC polynucleotides, e.g. library members, may be cloned or amplified on episomally replicable vectors, a multiplicity of said vectors may be transferred into a cell and homologously recombined to form shuffled library members in vivo in a plant cell, algae cell, fungal, yeast, or bacterial cell. Other cell types may be used, if desired.

OXOX and/or OXDC polynucleotides, e.g. library members, may not be fragmented, but may be cloned or amplified on an episomally replicable vector as a direct repeat or indirect (or inverted) repeat, which each repeat comprising a distinct species of OXOX and/or OXDC polynucleotide sequences, said vector may be transferred into a cell and homologously recombined by intra-vector or inter-vector recombination to form shuffled library members in vivo in a plant cell, or microorganism.

At least one parental polynucleotide sequence that encodes an OXOX of a fungus, such as for example and not limitation, a polynucleotide sequence, for example, gene or cDNA sequence from Ceriporiopsis subvermispora, among others having oxidase activity The parental OXOX polynucleotide may be subjected to mutagenesis and/or shuffling or combinations thereof to generate a population of mutagenized OXOX polynucleotides which have substantial sequence identity to the parental OXOX polynucleotide sequence. The population of mutagenized polynucleotides may be transferred into a population of host cells wherein the mutagenized polynucleotides are expressed and the resultant transformed host cell population (transformants) may be selected or screened for OXOX activity, maintained or increased, or a phenotype thereof.

A variety of suitable host cells for shuffling or determining OXOX sequences will be apparent to those skilled in the art. Any suitable host cell may be used so long as the host cell allows for the proper folding and processing of the OXOX. The host cell may be a plant cell, for example, Arabidopsis, soybean or an algae cell, fungal cell, yeast cell, or bacterial cell.

### Compositions

Alternatively, fragments of a polynucleotide that are useful as hybridization probes or PCR primers generally do not encode fragment proteins retaining biological activity. Thus, fragments of a nucleotide sequence may range from at least about 20 nucleotides, about 50 nucleotides, about 100 nucleotides, up to the full-length polynucleotide encoding the proteins employed in the invention.

In some examples, a fragment of an OXOX polynucleotide that encodes a biologically active portion of a known fungal OXOX protein employed in the invention will encode at least 15, 25, 30, 50, 75, 100, 125, 150, 175, 200, 225, 250, 275, 300, 325, 350, 375, 400, 425, or 450 contiguous amino acids, or up to the total number of amino acids present in a partial or full-length fungal OXOX protein, for example, 435, 436, 437 or 438 amino acids for SEQ ID NOS: 18, 19, 21, 22, 23, 24, 25, 26, 27, 28, 29, 30, 31, 32, 33, 34, 35, 36, 37, 66, 67, 68, 69, 70, 71, 72, 73, 74, 75, 76, 77, 78, 79, 80, 81, 82, 83, 84, 85, 86, 87, 88, 89, 90, 91, 92, 100, 101, 102, 103, 104, 105, or 106.

In some examples, a fragment of an OXOX polynucleotide that encodes a biologically active portion of a fungal OXOX protein employed in the invention will an OXOX variant protein employed in the invention will encode at least 15, 25, 30, 50, 75, 100, 125, 150, 175, 200, 225, 250, 275, 300, 325, 350, 375, 400, 425, or 450 contiguous amino acids, or up to the total number of amino acids present in a partial or full-length OXOX variant protein of the invention, for example, 435, 436, 437 or 438 amino acids for SEQ ID NOS: 18, 19, 21, 22, 23, 24, 25, 26, 27, 28, 29, 30, 31, 32, 33, 34, 35, 36, 37, 66, 67, 68, 69, 70, 71, 72, 73, 74, 75, 76, 77, 78, 79, 80, 81, 82, 83, 84, 85, 86, 87, 88, 89, 90, 91, 92, 100, 101, 102, 103, 104, 105, or 106.

A biologically active portion of an OXOX protein can be prepared by isolating a portion of one of the OXOX polynucleotides employed in the invention, expressing the encoded portion of the OXOX variant protein (e.g., by recombinant expression in vitro), and assessing the activity of the encoded portion of the OXOX protein. Polynucleotides that are fragments of an OXOX nucleotide sequence comprise at least 16, 20, 50, 75, 100, 150, 200, 250, 300, 350, 500, 550, 500, 550, 600, 650, 700, 800, 850, 900, 950, 1,000, 1050, 1,100, 1150, 1200, 1250, 1300 nucleotides, or up to the number of nucleotides present in a full-length OXOX variant polynucleotide disclosed herein or known fungal OXOX, for example, 1308, 1314, 1326, or 1368 nucleotides as exemplified by SEQ ID NOS: 1, 2, 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 58, 59, 60, 61, 62, 63, 64, 65, 93, 94, 95, 96, 97, 98, or 99.

“Variants” is intended to include substantially similar sequences. For polynucleotides, a variant comprises a deletion and/or addition of one or more nucleotides at one or more sites within the parental polynucleotide, e.g. a native polynucleotide from a fungus or plant, that may be codon-optimized, and/or a substitution of one or more nucleotides at one or more sites in the parental polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the OXOX variant polypeptides of the invention or known fungal OXOX or variants thereof. Naturally occurring variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode an OXOX variant protein employed in the invention. Generally, variants of a particular polynucleotide of the invention will have at least about 50%, 55%, 50%, 55%, 60%, 65%, 70%, 75%, 80%, 85%, 86%, 87%, 88%, 89%, 90%, 91%, 92%, 93%, 95%, 95%, 96%, 97%, 98%, 99% or more sequence identity to a particular reference polynucleotide, e.g., native OXOX polynucleotide or template OXOX polynucleotide or known fungal OXOX polynucleotide, as determined by sequence alignment programs and parameters described elsewhere herein.

Variants of a particular polynucleotide employed in the invention (i.e., the reference or parental polynucleotide) can also be evaluated by comparison of the sequence identity between the polypeptide encoded by a variant polynucleotide and the polypeptide encoded by the reference or parental polynucleotide. Thus, for example, an isolated polynucleotide that encodes a polypeptide with a given percent sequence identity to any one of the polypeptides of SEQ ID NOS: 18, 19, 21, 22, 23, 24, 25, 26, 27, 28, 29, 30, 31, 32, 33, 34, 66, 67, 68, 69, 70, 71, 72, 73, 74, 75, 76, 77, 78, 79, 80, 81, 82, 83, 84, 85, 86, 87, 88, 89, 90, 91, 92, 100, 101, 102, 103, 104, 105, or 106 is encompassed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 50%, 55%, 50%, 55%, 60%, 65%, 70%, 75%, 80%, 85%, 86%, 87%, 88%, 89%, 90%, 91%, 92%, 93%, 95%, 95%, 96%, 97%, 98%, 99% or more sequence identity. In some examples, OXOX variant polynucleotides of the invention can have at least about 50%, 55%, 60%, 65%, 70%, 75%, 80%, 85%, 86%, 87%, 88%, 89%, 90%, 91%, 92%, 93%, 95%, 95%, 96%, 97%, 98%, 99% or more sequence identity to any of the polynucleotides of SEQ ID NOS:1, 2, 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 59, 60, 61, 62, 63, 64, 65, 93, 94, 95, 96, 97, 98, or 99 and are encompassed by the invention. Also included are isolated polynucleotides that encode polypeptides having at least about 50%, 55%, 60%, 65%, 70%, 75%, 80%, 85%, 86%, 87%, 88%, 89%, 90%, 91%, 92%, 93%, 95%, 95%, 96%, 97%, 98%, 99% or more sequence identity to any of the polypeptides of SEQ ID NO: 18, 19, 21, 22, 23, 24, 25, 26, 27, 28, 29, 30, 31, 32, 33, 34, 35, 36, 37, 66, 67, 68, 69, 70, 71, 72, 73, 74, 75, 76, 77, 78, 79, 80, 81, 82, 83, 84, 85, 86, 87, 88, 89, 90, 91, 92, 100, 101, 102, 103, 104, 105, or 106.

“Variant” protein is intended to include a protein derived from the native or parental protein by deletion, substitution or addition of one or more amino acids at one or more sites in the native or parental protein and/or substitution of one or more amino acids at one or more sites in the native or parental protein. Variant proteins encompassed by the present invention are biologically active, that is they continue to possess the desired biological activity of the native or parental protein, that is, OXOX activity as described herein. Such variants may result from, for example, genetic polymorphism or from human manipulation. Biologically active OXOX variants of the invention will have at least about 50%, 55%, 60%, 65%, 70%, 75%, 80%, 85%, 86%, 87%, 88%, 89%, 90%, 91%, 92%, 93%, 95%, 95%, 96%, 97%, 98%, 99% or more sequence identity to the amino acid sequence for the native protein as determined by sequence alignment programs and parameters described elsewhere herein. A biologically active variant of a protein of the invention may differ from that protein by 50 or more amino acid residues, 30-50 residues, 15-30 amino acid residues, as few as 1-15 amino acid residues, as few as 1-10, such as 6-10, as few as 5, as few as 5, 3, 2, or even 1 amino acid residue. In some examples, OXOX variant polypeptides of the invention can have at least about 50%, 55%, 60%, 65%, 70%, 75%, 80%, 85%, 86%, 87%, 88%, 89%, 90%, 91%, 92%, 93%, 95%, 95%, 96%, 97%, 98%, 99% or more sequence identity to any of the polypeptides of SEQ ID NOS: 18, 19, 21, 22, 23, 24, 25, 26, 27, 28, 29, 30, 31, 32, 33, 34, 35, 36, 37, 66, 67, 68, 69, 70, 71, 72, 73, 74, 75, 76, 77, 78, 79, 80, 81, 82, 83, 84, 85, 86, 87, 88, 89, 90, 91, 92, 100, 101, 102, 103, 104, 105, or 106.

The proteins employed in the methods of the invention may be altered in various ways including amino acid substitutions, deletions, truncations, and insertions. Methods for such manipulations are generally known in the art. For example, amino acid sequence variants and fragments of the OXOX proteins, e.g. known fungal OXOX or OXOX variants, can be prepared by mutations in the DNA. Methods for mutagenesis and polynucleotide alterations are well known in the art. See, for example, Kunkel (1985) Proc. Natl. Acad. Sci. USA 82:588-592; Kunkel et al. (1987) Methods in Enzymol. 155:367-382; U.S. Pat. No. 5,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, are contemplated. Variants of OXOX polypeptides can also include isolating natural variants from plants or fungal cells that exist in nature or creating recombinant OXOX's.

Thus, the genes and polynucleotides employed in the invention include both the naturally-occurring sequences as well as mutant forms. Likewise, the proteins employed in the invention encompass naturally occurring proteins as well as variations and modified forms thereof. Such variants will continue to possess the desired OXOX activity. Obviously, the mutations that will be made in the DNA encoding the variant must not place the sequence out of reading frame and optimally will not create complementary regions that could produce secondary mRNA structure.

The deletions, insertions, and substitutions of the protein sequences encompassed herein may produce changes in the characteristics of the protein. However, when it is difficult to predict the exact effect of the substitution, deletion, or insertion in advance of doing so, one skilled in the art will appreciate that the effect will be evaluated by routine screening assays. That is, the activity and/or expression can be evaluated by in enzymes assays, real time RT-PCR analysis, Northern, Westerns, and the like. Assays for detecting such activity or expression are known to one skilled in the art. Alternately, they are described in detail elsewhere herein. For example, an oligonucleotide of at least 15, 30, 50, 100, 200, 300, 400, 500, 600, 700, 800, 900 or 1000 nucleotides in length and sufficient to specifically hybridize under stringent conditions to known fungal OXOX variant mRNA or known OXOX mRNA may be used in Northern blot analysis. OXOX variant proteins or known fungal OXOX proteins may be detected using a labeled antibody capable of binding to OXOX variant proteins of the present invention or known fungal OXOX proteins. Antibodies can be polyclonal, or more preferably, monoclonal. An isolated OXOX variant protein, an isolated known fungal OXOX protein, or fragment thereof can be used as an immunogen to generate antibodies that bind specifically to OXOX's of the present invention or known fungal OXOX using standard techniques for polyclonal and monoclonal antibody preparation. Techniques for detection of an OXOX variant protein or known fungal OXOX include enzyme linked immunosorbent assays (ELISAs), Western blots, immunoprecipitations and immunofluorescence.

Variant polynucleotides and proteins also encompass sequences and proteins derived from a mutagenic and recombinogenic procedure such as DNA shuffling. With such a procedure, one or more different OXOX coding sequences can be manipulated to create a new OXOX possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the OXOX gene of the invention or OXOX variant polynucleotide and other known OXOX or OXDC genes to obtain a new gene coding for a protein with an improved property of interest, such as a decreased Kₘ in the case of an enzyme or increased digestibility. Strategies for such DNA shuffling are known in the art. See, for example, Stemmer (1995) Proc. Natl. Acad. Sci. USA 91:10757-10751; Stemmer (1995) Nature 370:389-391; Crameri et al. (1997) Nature Biotech. 15:536-538; Moore et al. (1997) J. Mol. Biol. 272:336-357; Zhang et al. (1997) Proc. Natl. Acad. Sci. USA 95:5505-5509; Crameri et al. (1998) Nature 391:288-291; and U.S. Pat. Nos. 5,605,793 and 5,837,558.

The polynucleotides employed in the invention can be used to isolate corresponding sequences from other organisms, particularly other fungi. In this manner, methods such as PCR, hybridization, and the like can be used to identify such sequences based on their sequence homology to the sequences set forth herein. Sequences isolated based on their sequence identity to the entire OXOX sequences set forth in SEQ ID NOS: 1, 2, 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 58, 59, 60, 61, 62, 63, 64, 65, 93, 94, 95, 96, 97, 98, or 99 or to variants and fragments thereof are encompassed by the present invention. Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 60%, 70%, 75%, 80%, 85%, 86%, 87%, 88%, 89%, 90%, 91%, 92%, 93%, 95%, 95%, 96%, 97%, 98%, 99%, or greater sequence identity. Functions of orthologs are often highly conserved among species. Thus, isolated polynucleotides that encode an OXOX variant protein or known fungal OXOX protein and which hybridize under stringent conditions to the sequence of SEQ ID NOS: 1, 2, 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 58, 59, 60, 61, 62, 63, 64, 65, 93, 94, 95, 96, 97, 98, or 99 or to complements, variants, or fragments thereof, are encompassed by the present invention.

In a PCR approach, oligonucleotide primers can be designed for use in PCR reactions to amplify corresponding DNA sequences from cDNA or genomic DNA extracted from any organism of interest, such as a fungus or plant. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or another detectable marker. Thus, for example, probes for hybridization can be made by labeling synthetic oligonucleotides based on the OXOX variant polynucleotides of the invention or known fungal OXOX. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, an entire OXOX variant polynucleotide disclosed herein, or an entire known fungal OXOX polynucleotide, one or more portions thereof, may be used as a probe capable of specifically hybridizing to corresponding OXOX polynucleotide and messenger RNAs. To achieve specific hybridization under a variety of conditions, such probes include sequences that are unique among OXOX variant polynucleotide sequences or known fungal OXOX polynucleotide sequences and are optimally at least about 10 nucleotides in length, and most optimally at least about 20 nucleotides in length. Such probes may be used to amplify corresponding OXOX polynucleotide from a chosen organism, e.g. fungus or plant, by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 50 to 55% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 25 hours, usually about 5 to about 12 hours. The duration of the wash time will be at least a length of time sufficient to reach equilibrium.

Specificity is typically the function of post-hybridization washes, the critical factors being the ionic strength and temperature of the final wash solution. For DNA-DNA hybrids, the Tₘ can be approximated from the equation of Meinkoth and Wahl (1985) Anal. Biochem. 138:267-285: Tₘ=81.5° C.+16.6 (log M)+0.51 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 5° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 15, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 55° C. (aqueous solution) or 32° C. (formamide solution), it is optimal to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

The following terms are used to describe the sequence relationships between two or more polynucleotides or polypeptides: (a) “reference sequence”, (b) “comparison window”, (c) “sequence identity”, and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 50, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 5:11-17; the local alignment algorithm of Smith et al. (1981) Adv. Appl. Math. 2:582; the global alignment algorithm of Needleman and Wunsch (1970) J. Mol. Biol. 58:553-553; the search-for-local alignment method of Pearson and Lipman (1988) Proc. Natl. Acad. Sci. 85:2555-2558; the algorithm of Karlin and Altschul (1990) Proc. Natl. Acad. Sci. USA 872265, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA Accelrys GCG (Accelrys Inc., 9685 Scranton Road, San Diego, Calif., USA). Alignments using these programs can be performed using the default parameters. The CLUSTAL program is well described by Higgins et al. (1988) Gene 73:237-255 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1995) Meth. Mol. Biol. 25:307-331. The ALIGN program is based on the algorithm of Myers and Miller (1988) supra. A PAM120 weight residue table, a gap length penalty of 12, and a gap penalty of 5 can be used with the ALIGN program when comparing amino acid sequences. The BLAST programs of Altschul et al (1990) J. Mol. Biol. 215:503 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. The United States' National Center for Biotechnology Information and the European Bioinformatics Institute of the European Molecular Biology Laboratory provide such tools, as do various commercial entities known to those of skill in the art. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix.

GAP uses the algorithm of Needleman and Wunsch (1970) J. Mol. Biol. 58:553-553, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 5, 5, 6, 7, 8, 9, 10, 15, 20, 25, 30, 35, 50, 55, 50, 55, 60, 65 or greater.

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 5 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

### Methods

### I. Providing Sequences

The sequences of the present invention can be introduced and expressed in a host cell such as prokaryotic or eukaryotic cells, for example, fungi, bacteria, yeast, insect, mammalian, or optimally plant cells. It is expected that those of skill in the art are knowledgeable in the numerous systems available for the introduction of a polypeptide or a nucleotide sequence of the present invention into a host cell. No attempt to describe in detail the various methods known for providing proteins in prokaryotes or eukaryotes will be made.

By “host cell” is meant a cell which comprises a heterologous nucleic acid sequence of the invention. Host cells may be prokaryotic cells such as E. coli, or eukaryotic cells such as yeast, insect, amphibian, plant, or mammalian cells. Host cells can also be monocotyledonous or dicotyledonous plant cells. In one embodiment, the monocotyledonous host cell is a maize host cell. In one embodiment, the dicotyledonous host cell is a soybean host cell.

The use of the term “polynucleotide” is not intended to limit the present invention to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally-occurring molecules and synthetic analogues. The polynucleotides of the invention also encompass all forms of sequences including, but not limited to, single-stranded forms, double-stranded forms, hairpins, stem-and-loop structures, and the like.

A known fungal OXOX or an OXOX variant polynucleotide employed of the invention can be provided in expression cassettes for expression in the plant of interest. The cassette will include 5′ and 3′ regulatory sequences operably linked to an OXOX variant polynucleotide. “Operably linked” is intended to mean a functional linkage between two or more elements. For example, an operable linkage between a polynucleotide of interest and a promoter is a functional link that allows for expression of the polynucleotide of interest. Operably linked elements may be contiguous or non-contiguous. When used to refer to the joining of two protein coding regions, operably linked means that the coding regions are in the same reading frame.

The cassette may also include a polynucleotide encoding a secretion signal. A number of suitable secretion signal sequences are known in the art and may be used with the known fungal OXOX or OXOX variant's of the present invention. The secretion signal sequence can be an RNA leader which directs secretion of the subsequently transcribed protein or polypeptide, or the secretion signal can be a carboxy or amino terminal peptide sequence that is recognized by a host plant secretory pathway. The secretion signal may target the protein to a desired location within the plant or plant cell, for example, cytosol, endoplasmic reticulum (ER), vacuole, or chloroplast or other desired locations. The polynucleotide encoding a secretion signal can be positioned between the promoter and the known fungal OXOX polynucleotide encoding the OXOX or OXOX polynucleotide encoding the OXOX variant, using known molecular cloning techniques as indicated above.

According to one embodiment, a signal sequence such as BAA SS is included before the sequence encoding the OXOX variant polypeptide or mature OXOX protein, for example, barley alpha amylase (BAA SS). The BAA SS polynucleotide may have a nucleotide sequence corresponding to SEQ ID NO: 3 and a BAA SS polypeptide having the amino acid sequence of SEQ ID NO: 20. Other exemplary secretion signals include a chloroplast targeting peptide (CTP) such as CTP1 (David R. Corbin et al. Expression and Chloroplast Targeting of Cholesterol Oxidase in Transgenic Tobacco Plants. Plant Physiol. (2001) 126: 1116-1128), a murine kappa light chain signal peptide (Schouten, A et al. (1996) The C-terminal KDEL sequence increases the expression level of a single-chain antibody designed to be targeted to both the cytosol and the secretory pathway in transgenic tobacco. Plant Mol. Biol. 30, 781-793), a C-terminal KDEL sequence (Schouten, A et al. (1996) The C-terminal KDEL sequence increases the expression level of a single-chain antibody designed to be targeted to both the cytosol and the secretory pathway in transgenic tobacco. Plant Mol. Biol. 30, 781-793), a C-terminal propeptide from Concanavalin A (Claude S J et al. (2005) Targeting of proConA to the plant vacuole depends on its nine amino-acid C-terminal propeptide. Plant Cell Physiol 46: 1603-1612), and others such as Arabidopsis alpha-carbonic anhydrase (CAH1) (L. Faye and H. Daniell (2006) Novel pathways for glycoprotein import into chloroplasts Plant Biotechnology Journal 4:275-279) and the like.

The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the known fungal OXOX polynucleotide or OXOX variant polynucleotide to be under the transcriptional regulation of the regulatory regions. The expression cassette may additionally contain selectable marker genes.

The expression cassette will include, in the 5′-3′ direction of transcription, a transcriptional and translational initiation region (i.e., a promoter), the known fungal OXOX polynucleotide or an OXOX variant polynucleotide of the invention, and a transcriptional and translational termination region (i.e., termination region) functional in plants. The regulatory regions (including promoters, transcriptional regulatory regions, and translational termination regions) may be native/analogous to the host cell and/or to an OXOX polynucleotide or each other. Alternatively, the regulatory regions, the known fungal OXOX polynucleotide, and/or OXOX variant polynucleotide of the invention may be foreign/heterologous to the host cell and/or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a promoter that is heterologous to the coding sequence.

While it may be optimal to express the sequences using heterologous promoters, a native promoter sequences of the known fungal OXOX, parental OXOX of the variant, or a native promoter sequences of a plant OXOX may be used. Such constructs can change the expression levels of the OXOX in the plant or plant cell. Thus, the phenotype of the plant or plant cell can be altered.

The termination region may be native with the transcriptional initiation region of an OXOX polynucleotide, novel OXOX variant polynucleotide or known OXOX polynucleotide, may be native with the operably linked OXOX polynucleotide of interest, may be native with the plant host, or may be derived from another source (i.e., foreign or heterologous) to the promoter, the OXOX polynucleotide of interest, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:151-155; Proudfoot (1991) Cell 65:671-675; Sanfacon et al. (1991) Genes Dev. 5:151-159; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be optimized for increased expression in the transformed plant by using plant-preferred codons. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,536,391, and Murray et al. (1989) Nucleic Acids Res. 17:577-598, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 155:9-20), and human immunoglobulin heavy-chain binding protein (BiP) (Macejak et al. (1991) Nature 353:90-95); untranslated leader from the coat protein mRNA of alfalfa mosaic virus (AMV RNA 5) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 85:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,5-dichlorophenoxyacetate (2,5-D). Additional selectable markers include phenotypic markers such as β-galactosidase and fluorescent proteins such as green fluorescent protein (GFP) (Su et al. (2005) Biotechnol Bioeng 85:610-9 and Fetter et al. (2005) Plant Cell 16:215-28), cyan florescent protein (CYP) (Bolte et al. (2005) J. Cell Science 117:953-55 and Kato et al. (2002) Plant Physiol 129:913-52), and yellow florescent protein (PhiYFP™ from Evrogen, see, Bolte et al. (2005) J. Cell Science 117:953-55). For additional selectable markers, see generally, Yarranton (1992) Curr. Opin. Biotech. 3:506-511; Christopherson et al. (1992) Proc. Natl. Acad. Sci. USA 89:6315-6318; Yao et al. (1992) Cell 71:63-72; Reznikoff (1992) Mol. Microbiol. 6:2519-2522; Barkley et al. (1980) in The Operon, pp. 177-220; Hu et al. (1987) Cell 58:555-566; Brown et al. (1987) Cell 59:603-612; Figge et al. (1988) Cell 52:713-722; Deuschle et al. (1989) Proc. Natl. Acad. Sci. USA 86:5500-5505; Fuerst et al. (1989) Proc. Natl. Acad. Sci. USA 86:2559-2553; Deuschle et al. (1990) Science 258:580-583; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3353-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:5657-5653; Hillenand-Wissman (1989) Topics Mol. Struc. Biol. 10:153-162; Degenkolb et al. (1991) Antimicrob. Agents Chemother. 35:1591-1595; Kleinschnidt et al. (1988) Biochemistry 27:1095-1105; Bonin (1993) Ph.D. Thesis, University of Heidelberg; Gossen et al. (1992) Proc. Natl. Acad. Sci. USA 89:5557-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 335:721-725. Such disclosures are herein incorporated by reference. The above list of selectable marker genes is not meant to be limiting. Any selectable marker gene can be used in the present invention.

A number of promoters can be used in the practice of the invention, including the native promoter of the polynucleotide sequence of interest. The promoters can be selected based on the desired outcome. The nucleic acids can be combined with constitutive, tissue-preferred, inducible, or other promoters for expression in plants.

Such constitutive promoters include, for example, the core promoter of the Rsyn7 promoter and other constitutive promoters disclosed in WO 99/53838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1985) EMBO J. 3:2723-2730); ALS promoter (U.S. Pat. No. 5,659,026), and the like. Other constitutive promoters include, for example, those disclosed in U.S. Pat. Nos. 5,608,159; 5,608,155; 5,605,121; 5,569,597; 5,566,785; 5,399,680; 5,268,563; 5,608,152; and 6,177,611.

Many different constitutive promoters can be utilized in the instant invention to express a known fungal OXOX polynucleotide that encodes an OXOX or an OXOX polynucleotide that encodes an OXOX variant. Examples include promoters from plant viruses such as the 35S promoter from cauliflower mosaic virus (CaMV), as described in Odell, et al., Nature, 313: 810-812 (1985), and hereby incorporated by reference, and promoters from genes such as rice actin (McElroy, et al., Plant Cell, 163-171 (1990)); ubiquitin (Christensen, et al., Plant Mol. Biol., 12: 619-632 (1992); and Christensen, et al., Plant Mol. Biol., 18: 675-689 (1992)); pEMU (Last, et al., Theor. Appl. Genet., 81: 581-588 (1991)); MAS (Velten, et al., EMBO J., 3: 2723-2730 (1984)); maize H3 histone (Lepetit, et al., Mol. Gen. Genet., 231: 276-285 (1992); and Atanassvoa, et al., Plant Journal, 2(3): 291-300 (1992)), the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, ALS promoter, as described in published PCT Application WO 96/30530, a synthetic promoter, such as, Rsyn7, SCP and UCP promoters as described in U.S. patent application Ser. No. 09/028,819, now U.S. Pat. No. 6,072,050 filed Feb. 24, 1998 and herein incorporated by reference, and other transcription initiation regions from various plant genes known to those of skill.

Many different inducible promoters can be utilized in the instant invention to express a known fungal OXOX polynucleotide that encodes an OXOX or an OXOX variant polynucleotide that encodes an OXOX variant. Examples include pathogen-inducible promoters. Advantageously use of a pathogen-inducible promoter allows for expression of the fungal OXOX or OXOX variant only when a plant is infected or otherwise encounters a pathogen. Pathogen-inducible promoters may comprise those promoters or regulatory sequences from genes which are induced as a consequence of infection by pathogens, such as, for example, Sclerotinia, genes of PR proteins, SAR proteins, beta-1,3-glucanase, chitinase and the like (for example Redolfi et al. (1983) Neth J Plant Pathol 89:245-254; Uknes, et al. (1992) The Plant Cell 4:645-656; Van Loon (1985) Plant Mol Viral 4:111-116; Marineau et al. (1987) Plant Mol Biol 9:335-342; Matton et al. (1987) Molecular Plant-Microbe Interactions 2:325-342; Somssich et al. (1986) Proc Natl Acad Sci USA 83:2427-2430; Somssich et al. (1988) Mol Gen Genetics 2:93-98; Chen et al. (1996) Plant J 10:955-966; Zhang and Sing (1994) Proc Natl Acad Sci USA 91:2507-2511; Warner, et al. (1993) Plant J 3:191-201; Siebertz et al. (1989) Plant Cell 1:961-968 (1989). Also comprised are wounding-inducible promoters such as that of the win3.12T promoter or regions thereof (Yevtushenko D P, Sidorov V A, Romero R, Kay, W W and Santosh M Wound-inducible promoter from poplar is responsive to fungal infection in transgenic potato. Plant Science. (2004) 167:715-724) the pinII gene (Ryan (1990) Ann Rev Phytopath 28:425-449; Duan et al. (1996) Nat Biotech 14:494-498), of the wun1 and wun2 gene (U.S. Pat. No. 5,428,148), of the win1 and win2 gene (Stanford et al. (1989) Mol Gen Genet. 215:200-208), of system in (McGurl et al. (1992) Science 225:1570-1573), of the WIP1 gene (Rohmeier et al. (1993) Plant Mol Biol 22:783-792; Eckelkamp et al. (1993). FEBS Letters 323:73-76), of the MPI gene (Corderok et al. (1994) Plant J 6(2):141-150), the hsr203J, str246C, sgd24 tobacco promoters (Malnoy M et al. Activation of three pathogen-inducible promoters of tobacco in transgenic pear (Pyrus communis L.) after abiotic and biotic elicitation. (2003) Planta. 216:802-814) and the like. A source of further pathogen-inducible promoters is the PR gene family. A number of elements in these promoters have been found to be advantageous. Thus, the region -364 to −288 in the promoter of PR-2d provides salicylate specificity (Buchel et al. (1996) Plant Mol Biol 30, 493-504). The sequence 5′-TCATCTTCTT-3′ (SEQ ID NO:38) is encountered repeatedly in the promoter of barley beta-1,3-glucanase and more than 30 further stress-induced genes. In tobacco, this region binds a nuclear protein whose abundance is increased by salicylate. The PR-1 promoters from tobacco and Arabidopsis (EP-A 0 332 104, WO 98/03536) are likewise suitable for use as pathogen-inducible promoters. “Acidic PR-5”-(aPR5)-promoters from barley (Schweizer et al. (1997) Plant Physiol 114:79-88) and wheat (Rebmann et al. (1991) Plant Mol Biol 16:329-331) are preferred, since they are particularly specifically pathogen-induced. aPR5 proteins accumulate within about 4 to 6 hours after pathogen attack and have only very limited background expression (WO 99/66057). One approach to achieve higher pathogen-induced specificity is the preparation of synthetic promoters from combinations of known pathogen-responsive elements (Rushton et al. (2002) Plant Cell 14, 749-762; WO 00/01830; WO 99/66057). Further pathogen-inducible promoters from different species are known to the person skilled in the art (EP-A 1 165 794; EP-A 1 062 356; EP-A 1 041 148; EP-A 1 032 684. OXOX's that have maintained or increased OXOX activity in a host cell may be identified by transforming a host cell with a polynucleotide encoding the known fungal OXOX or OXOX variant to obtain a transformant. The host cells comprising polynucleotides encoding the known fungal OXOX's or OXOX variants may be screened to isolate or identify host cells and/or their progeny which express OXOX(s) having the desired enhanced phenotype. For example, host cells such as E. coli or plant cells comprising the known fungal OXOX's or variant OXOX's encoding sequences may be identified for those having OXOX activity, using, for example, in vitro colorimetric or kinetic assays.

Oxidase enzyme assays (Suigura, et al., Chem. Pharm. Bull., 27(9): 2003-2007 (1979)) herein incorporated by reference may also be performed on a sample from a leaf or petiole of a plant transformed with OXOX variants of the present invention. The amount of hydrogen peroxide in the media of each sample may be determined at a desired time point and the values of various samples and plants compared. For example, an increased hydrogen peroxide level of a sample from an OXOX variant relative to the level of hydrogen peroxide from a control OXOX would be indicative of increased OXOX activity. Other suitable assays may be used to determine OXOX activity, including but not limited to, Synthetic Gastric Fluid (SGF) assay. See, for example, Example 9 herein.

In addition, the constructs may contain control regions that regulate as well as engender expression. Generally, in accordance with many commonly practiced procedures, such regions will operate by controlling transcription, such as transcription factors, repressor binding sites and termination signals, among others. For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Transcription of the DNA encoding the polypeptides of the present invention by higher eukaryotes may be increased by inserting an enhancer sequence into the vector Enhancers are cis-acting elements of DNA, usually about from 10 to 300 bp, that act to increase transcriptional activity of a promoter in a given host cell-type. Examples of enhancers include the SV40 enhancer, which is located on the late side of the replication origin at by 100 to 270, the cytomegalovirus early promoter enhancer, the polyoma enhancer on the late side of the replication origin, and adenovirus enhancers. Additional enhancers useful in the invention to increase transcription of the introduced DNA segment, include, inter alia, viral enhancers like those within the 35S promoter, as shown by Odell et al. (1988) Plant Mol. Biol. 10:263-72, and an enhancer from an opine gene as described by Fromm et al. (1989) Plant Cell 1:977. The enhancer may affect the tissue-specificity and/or temporal specificity of expression of sequences included in the vector.

Termination regions also facilitate effective expression by ending transcription at appropriate points. Useful terminators for practicing this invention include, but are not limited to, pinII (See An et al. (1989) Plant Cell 1(1):115-122), glb1 (See Genbank Accession #L22345), gz (See gzw64a terminator, Genbank Accession #S78780), and the nos terminator from Agrobacterium.

The methods of the invention involve introducing a polypeptide or polynucleotide into a plant. “Introducing” is intended to mean presenting to the plant the polynucleotide or polypeptide in such a manner that the sequence gains access to the interior of a cell of the plant. The methods of the invention do not depend on a particular method for introducing a sequence into a plant, only that the polynucleotide or polypeptides gains access to the interior of at least one cell of the plant. Methods for introducing polynucleotide or polypeptides into plants are known in the art including, but not limited to, stable transformation methods, transient transformation methods, and virus-mediated methods.

“Stable transformation” is intended to mean that the nucleotide construct introduced into a plant integrates into the genome of the plant and is capable of being inherited by the progeny thereof “Transient transformation” is intended to mean that a polynucleotide is introduced into the plant and does not integrate into the genome of the plant or a polypeptide is introduced into a plant.

Transformation protocols as well as protocols for introducing polypeptides or polynucleotide sequences into plants may vary depending on the type of plant or plant cell, i.e., monocot or dicot, targeted for transformation.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25855, WO99/25850, WO99/25855, and WO99/25853, all of which are herein incorporated by reference. Briefly, the polynucleotide of the invention can be contained in transfer cassette flanked by two non-identical recombination sites. The transfer cassette is introduced into a plant have stably incorporated into its genome a target site which is flanked by two non-identical recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick et al. (1986) Plant Cell Reports 5:81-85. These plants may then be pollinated with either the same transformed strain or different strains, and the resulting progeny having desired expression of the phenotypic characteristic of interest can be identified. Two or more generations may be grown to ensure that expression of the desired phenotypic characteristic is stably maintained and inherited, and then seeds can be harvested to ensure expression of the desired phenotypic characteristic has been achieved. In this manner, the present invention provides a transformed seed (also referred to as a “transgenic seed”) having a polynucleotide of the invention, for example, an expression cassette of the invention, stably incorporated into its genome.

Pedigree breeding generally starts with the crossing of two genotypes, such as an elite line of interest and one other line having one or more desirable characteristics (e.g., having stably incorporated a polynucleotide of the invention, having a modulated activity and/or level of the polypeptide of the invention) which complements the elite line of interest. If the two original parents do not provide all the desired characteristics, other sources can be included in the breeding population. In the pedigree method, superior plants are selfed and selected in successive filial generations. In the succeeding filial generations the heterozygous condition gives way to homogeneous lines as a result of self-pollination and selection. Typically in the pedigree method of breeding, five or more successive filial generations of selfing and selection are practiced: F1→F2; F2→F3; F3→F4; F4→F5, etc. After a sufficient amount of inbreeding, successive filial generations will serve to increase seed of the developed inbred. Preferably, the inbred line comprises homozygous alleles at about 95% or more of its loci.

In addition to being used to create a backcross conversion, backcrossing can also be used in combination with pedigree breeding to modify an elite line of interest and a hybrid that is made using the modified elite line. As discussed previously, backcrossing can be used to transfer one or more specifically desirable traits from one line, the donor parent, to an inbred called the recurrent parent, which has overall good agronomic characteristics yet lacks that desirable trait or traits. However, the same procedure can be used to move the progeny toward the genotype of the recurrent parent but at the same time retain many components of the non-recurrent parent by stopping the backcrossing at an early stage and proceeding with selfing and selection. For example, an F1, such as a commercial hybrid, is created. This commercial hybrid may be backcrossed to one of its parent lines to create a BC1 or BC2. Progeny are selfed and selected so that the newly developed inbred has many of the attributes of the recurrent parent and yet several of the desired attributes of the non-recurrent parent. This approach leverages the value and strengths of the recurrent parent for use in new hybrids and breeding.

Therefore, an embodiment of this invention is a method of making a backcross conversion of maize inbred line of interest, comprising the steps of crossing a plant of maize inbred line of interest with a donor plant comprising a mutant gene or transgene conferring a desired trait (i.e., maintained or increased OXOX activity), selecting an F1 progeny plant comprising the mutant gene or transgene conferring the desired trait, and backcrossing the selected F1 progeny plant to the plant of maize inbred line of interest. This method may further comprise the step of obtaining a molecular marker profile of maize inbred line of interest and using the molecular marker profile to select for a progeny plant with the desired trait and the molecular marker profile of the inbred line of interest. In the same manner, this method may be used to produce an F1 hybrid seed by adding a final step of crossing the desired trait conversion of maize inbred line of interest with a different maize plant to make F1 hybrid maize seed comprising a mutant gene or transgene conferring the desired trait.

Recurrent selection is a method used in a plant breeding program to improve a population of plants. The method entails individual plants cross pollinating with each other to form progeny. The progeny are grown and the superior progeny selected by any number of selection methods, which include individual plant, half-sib progeny, full-sib progeny, selfed progeny and topcrossing. The selected progeny are cross-pollinated with each other to form progeny for another population. This population is planted and again superior plants are selected to cross pollinate with each other. Recurrent selection is a cyclical process and therefore can be repeated as many times as desired. The objective of recurrent selection is to improve the traits of a population. The improved population can then be used as a source of breeding material to obtain inbred lines to be used in hybrids or used as parents for a synthetic cultivar. A synthetic cultivar is the resultant progeny formed by the intercrossing of several selected inbreds.

Mass selection is a useful technique when used in conjunction with molecular marker enhanced selection. In mass selection seeds from individuals are selected based on phenotype and/or genotype. These selected seeds are then bulked and used to grow the next generation. Bulk selection requires growing a population of plants in a bulk plot, allowing the plants to self-pollinate, harvesting the seed in bulk and then using a sample of the seed harvested in bulk to plant the next generation. Instead of self pollination, directed pollination could be used as part of the breeding program.

Mutation breeding is one of many methods that could be used to introduce new traits into an elite line. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation; such as X-rays, Gamma rays (e.g. cobalt 60 or cesium 137), neutrons, (product of nuclear fission by uranium 235 in an atomic reactor), Beta radiation (emitted from radioisotopes such as phosphorus 32 or carbon 15), or ultraviolet radiation (preferably from 2500 to 2900 nm), or chemical mutagens (such as base analogues (5-bromo-uracil), related compounds (8-ethoxy caffeine), antibiotics (streptonigrin), alkylating agents (sulfur mustards, nitrogen mustards, epoxides, ethylenamines, sulfates, sulfonates, sulfones, lactones), azide, hydroxylamine, nitrous acid, or acridines. Once a desired trait is observed through mutagenesis the trait may then be incorporated into existing germplasm by traditional breeding techniques, such as backcrossing. Details of mutation breeding can be found in “Principles of Cultivar Development” Fehr, 1993, Macmillan Publishing Company, the disclosure of which is incorporated herein by reference. In addition, mutations created in other lines may be used to produce a backcross conversion of elite lines that comprise such mutations.

The present invention may be used for transformation of any plant species, including, but not limited to, monocots and dicots. Examples of plant species of interest include, but are not limited to, corn (Zea mays, also known as maize), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). In specific embodiments, plants of the present invention are crop plants (for example, corn, alfalfa, sunflower, Brassica, soybean, cotton, safflower, peanut, sorghum, wheat, millet, tobacco, etc.). In other embodiments, corn and soybean plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

Typically, an intermediate host cell will be used in the practice of this invention to increase the copy number of the cloning vector. With an increased copy number, the vector containing the nucleic acid of interest can be isolated in significant quantities for introduction into the desired plant cells. In one embodiment, plant promoters that do not cause expression of the polypeptide in bacteria are employed.

Prokaryotes most frequently are represented by various strains of E. coli; however, other microbial strains may also be used. Commonly used prokaryotic control sequences which are defined herein to include promoters for transcription initiation, optionally with an operator, along with ribosome binding sequences, include such commonly used promoters as the beta lactamase (penicillinase) and lactose (lac) promoter systems (Chang et al. (1977) Nature 198:1056), the tryptophan (trp) promoter system (Goeddel et al. (1980) Nucleic Acids Res. 8:5057) and the lambda derived P L promoter and N-gene ribosome binding site (Shimatake et al. (1981) Nature 292:128). The inclusion of selection markers in DNA vectors transfected in E coli. is also useful. Examples of such markers include genes specifying resistance to ampicillin, tetracycline, or chloramphenicol.

The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella (Palva et al. (1983) Gene 22:229-235); Mosbach et al. (1983) Nature 302:553-555).

A variety of eukaryotic expression systems such as yeast, insect cell lines, fungal, plant and mammalian cells, are known to those of skill in the art. As explained briefly below, a polynucleotide of the present invention can be expressed in these eukaryotic systems. In some embodiments, transformed/transfected plant cells, as discussed infra, are employed as expression systems for production of the proteins of the instant invention.

Synthesis of heterologous polynucleotides in yeast is well known (Sherman et al. (1982) Methods in Yeast Genetics, Cold Spring Harbor Laboratory). Two widely utilized yeasts for production of eukaryotic proteins are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

A protein of the present invention, once expressed, can be isolated from yeast by lysing the cells and applying standard protein isolation techniques to the lists. The monitoring of the purification process can be accomplished by using Western blot techniques or radioimmunoassay of other standard immunoassay techniques.

The sequences of the present invention can also be ligated to various expression vectors for use in transfecting cell cultures of, for instance, mammalian, fungal, insect, or plant origin. Illustrative cell cultures useful for the production of the peptides are mammalian cells. A number of suitable host cell lines capable of expressing intact proteins have been developed in the art, and include the HEK293, BHK21, and CHO cell lines. Expression vectors for these cells can include expression control sequences, such as an origin of replication, a promoter (e.g. the CMV promoter, a HSV tk promoter or pgk (phosphoglycerate kinase) promoter), an enhancer (Queen et al. (1986) Immunol. Rev. 89:59), and necessary processing information sites, such as ribosome binding sites, RNA splice sites, polyadenylation sites (e.g., an SV50 large T Ag poly A addition site), and transcriptional terminator sequences. Other animal cells useful for production of proteins of the present invention are available, for instance, from the American Type Culture Collection.

Appropriate vectors for expressing proteins of the present invention in insect cells are usually derived from the SF9 baculovirus. Suitable insect cell lines include mosquito larvae, silkworm, armyworm, moth and Drosophila cell lines such as a Schneider cell line (See, Schneider (1987) J. Embryol. Exp. Morphol. 27:353-365).

As with yeast, when higher animal or plant host cells are employed, polyadenylation or transcription terminator sequences are typically incorporated into the vector. An example of a terminator sequence is the polyadenylation sequence from the bovine growth hormone gene. Sequences for accurate splicing of the transcript may also be included. An example of a splicing sequence is the VP1 intron from SV50 (Sprague et al. (1983) J. Virol. 55:773-781). Additionally, gene sequences to control replication in the host cell may be incorporated into the vector such as those found in bovine papilloma virus type-vectors (Saveria-Campo (1985) DNA Cloning Vol. II a Practical Approach, D. M. Glover, Ed., IRL Press, Arlington, Va., pp. 213-238).

Animal and lower eukaryotic (e.g., yeast) host cells are competent or rendered competent for transfection by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextrin, electroporation, biolistics, and micro-injection of the DNA directly into the cells. The transfected cells are cultured by means well known in the art (Kuchler (1997) Biochemical Methods in Cell Culture and Virology, Dowden, Hutchinson and Ross, Inc.).

In certain embodiments the nucleic acid sequences of the known fungal OXOX or sequences of the present invention can be stacked with any combination of polynucleotide sequences of interest in order to create plants with a desired phenotype. The combinations generated may include multiple copies of any one of the polynucleotides of interest. For example, a known fungal OXOX polynucleotide or a polynucleotide of the present invention may be stacked with any other polynucleotide(s) of the present invention. The polynucleotides can also be stacked with any other gene or combination of genes involved in disease resistance including for example, polynucleotides involved in antifungal activities degradation of oxalate. Thus, in one aspect of the invention, a known fungal OXOX polynucleotide or an OXOX variant polynucleotide of the present invention is stacked with one or more an antifungal proteins, defensins, oxidoreductases or oxalate decarboxylases or combinations thereof.

The known fungal OXOX polynucleotides or OXOX variant polynucleotides of the present invention can also be stacked with any other gene or combination of genes to produce plants with a variety of desired trait combinations including but not limited to traits desirable for animal feed such as high oil genes (e.g., U.S. Pat. No. 6,232,529); balanced amino acids (e.g. hordothionins (U.S. Pat. Nos. 5,990,389; 5,885,801; 5,885,802; and 5,703,409); barley high lysine (Williamson et al. (1987) Eur. J. Biochem. 165:99-106; and WO 98/20122); and high methionine proteins (Pedersen et al. (1986) J. Biol. Chem. 261:6279; Kirihara et al. (1988) Gene 71:359; and Musumura et al. (1989) Plant Mol. Biol. 12: 123)); increased digestibility (e.g., modified storage proteins (U.S. application Ser. No. 10/053,410, filed Nov. 7, 2001); and thioredoxins (U.S. application Ser. No. 10/005,429, filed Dec. 3, 2001)), the disclosures of which are herein incorporated by reference. The polynucleotides of the present invention can also be stacked with traits desirable for insect, disease or herbicide resistance (e.g., Bacillus thuringiensis toxic proteins (U.S. Pat. Nos. 5,366,892; 5,747,450; 5,737,514; 5,723,756; 5,593,881; Geiser et at (1986) Gene 48:109); lectins (Van Damme et al. (1994) Plant Mol. Biol. 24:825); fumonisin detoxification genes (U.S. Pat. No. 5,792,931); avirulence and disease resistance genes (Jones et al. (1994) Science 266:789; Martin et al. (1993) Science 262:1432; Mindrinos et al. (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)); and traits desirable for processing or process products such as high oil (e.g., U.S. Pat. No. 6,232,529); modified oils (e.g., fatty acid desaturase genes (U.S. Pat. No. 5,952,544; WO 94/11516)); modified starches (e.g., ADPG pyrophosphorylases (AGPase), starch synthases (SS), starch branching enzymes (SBE) and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert et al. (1988) J. Bacteriol. 170:5837-5847) facilitate expression of polyhydroxyalkanoates (PHAs)), the disclosures of which are herein incorporated by reference. One could also combine the polynucleotides of the present invention with polynucleotides affecting agronomic traits such as male sterility, stalk strength, flowering time, or transformation technology traits such as cell cycle regulation or gene targeting (e.g. WO 99/61619; WO 00/17364; WO 99/25821).

These stacked combinations can be created by any method including but not limited to cross breeding plants by any conventional or TopCross methodology, or genetic transformation. If the traits are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in the plant.

II. Modulating the Concentration and/or Activity of an OXOX Variant Polypeptide or Known Fungal OXOX Polypeptide

A method for modulating the concentration and/or activity of a polypeptide of the present invention or known fungal OXOX in a plant is provided. In general, concentration and/or activity is increased or decreased by at least 1%, 5%, 10%, 20%, 30%, 50%, 50%, 60%, 70%, 80%, or 90% relative to a control plant, plant part, or cell, such as a native control plant, plant part, or cell. Modulation in the present invention may occur at any desired stage of development. In specific embodiments, the polypeptides of the present invention or known fungal OXOX polypeptides are modulated in monocots, particularly maize. In other embodiments, OXOX variant polypeptides of the present invention or known fungal OXOX polypeptides are modulated in dicots, particularly soybean.

A “subject plant or plant cell” is one in which genetic alteration, such as transformation, has been affected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

The expression level of the OXOX variant polypeptide or known fungal OXOX polypeptide may be measured directly, for example, by assaying for the level of the OXOX variant polypeptide or known fungal OXOX polypeptide in the plant, or indirectly, for example, by measuring the OXOX activity of the OXOX variant polypeptide or known fungal OXOX polypeptide in the plant. Methods for determining the OXOX activity are described elsewhere herein and include evaluation of phenotypic changes, such as increased disease resistance to an oxidase secreting pathogen or increased digestibility.

In specific embodiments, the OXOX variant polynucleotide or polypeptide of the invention or known fungal OXOX polynucleotide or polypeptide is introduced into the plant cell. Subsequently, a plant cell having the introduced sequence is selected using methods known to those of skill in the art such as, but not limited to, Southern blot analysis, DNA sequencing, PCR analysis, or phenotypic analysis. A plant or plant part altered by the foregoing embodiments is grown under plant forming conditions for a time sufficient to allow modulation of the concentration and/or activity of polypeptides of the present invention or known OXOX polypeptides in the plant. Plant forming conditions are well known in the art and are discussed briefly elsewhere herein.

It is also recognized that the level and/or activity of the polypeptide may be modulated by employing a polynucleotide that is not capable of directing, in a transformed plant, the expression of a protein or an RNA. For example, the polynucleotides of the invention or known OXOX polynucleotides may be used to design polynucleotide constructs that can be employed in methods for altering or mutating a genomic nucleotide sequence in an organism. Such polynucleotide constructs include, but are not limited to, RNA:DNA vectors, RNA:DNA mutational vectors, RNA:DNA repair vectors, mixed-duplex oligonucleotides, self-complementary RNA:DNA oligonucleotides, and recombinogenic oligonucleobases. Such nucleotide constructs and methods of use are known in the art. See, U.S. Pat. Nos. 5,565,350; 5,731,181; 5,756,325; 5,760,012; 5,795,972; and 5,871,985; all of which are herein incorporated by reference. See also, WO 98/59350, WO 99/07865, WO 99/25821, and Beetham et al. (1999) Proc. Natl. Acad. Sci. USA 96:8775-8778; herein incorporated by reference.

It is therefore recognized that methods of the present invention do not depend on the incorporation of the entire polynucleotide into the genome, only that the plant or cell thereof is altered as a result of the introduction of the polynucleotide into a cell. In one embodiment of the invention, the genome may be altered following the introduction of the polynucleotide into a cell. For example, the polynucleotide, or any part thereof, may be incorporated into the genome of the plant. Alterations to the genome of the present invention include, but are not limited to, additions, deletions, and substitutions of nucleotides into the genome. While the methods of the present invention do not depend on additions, deletions, and substitutions of any particular number of nucleotides, it is recognized that such additions, deletions, or substitutions comprise at least one nucleotide.

A. Increasing the Activity and/or Level of an OXOX Variant Polypeptide or Known Fungal OXOX Polypeptide

Methods are provided to increase the activity and/or level of an OXOX variant polypeptide or known fungal OXOX polypeptide. An increase in the level and/or activity of the OXOX variant polypeptide of the invention can be achieved by providing to the plant an OXOX variant polypeptide of the invention or known fungal OXOX polypeptide. The OXOX variant polypeptide or known fungal OXOX polypeptide can be provided by introducing the amino acid sequence encoding the OXOX variant polypeptide or known fungal OXOX polypeptide respectively into the plant, introducing into the plant a nucleotide sequence encoding an OXOX variant polypeptide or known fungal OXOX polypeptide, or alternatively, by modifying a genomic locus encoding an OXOX polypeptide.

As discussed elsewhere herein, many methods are known in the art for providing a polypeptide to a plant including, but not limited to, direct introduction of the polypeptide into the plant, introducing into the plant (transiently or stably) a polynucleotide construct encoding a polypeptide having OXOX activity. It is also recognized that the methods of the invention may employ a polynucleotide that is not capable of directing, in the transformed plant, the expression of a protein or an RNA. Thus, the level and/or activity of an OXOX variant polypeptide or known fungal OXOX polypeptide may be increased by altering the gene encoding an OXOX polypeptide or its promoter. See, e.g., Kmiec, U.S. Pat. No. 5,565,350; Zarling et al., PCT/US93/03868. Therefore mutagenized plants that carry mutations in OXOX genes, where the mutations increase expression of the OXOX gene or increase the OXOX activity of the encoded OXOX variant polypeptide or known fungal OXOX polypeptide are provided.

B. Reducing the Activity and/or Level of an OXOX Variant Polypeptide or Known Fungal OXOX Polypeptide

Methods are provided to reduce or eliminate the activity of an OXOX variant polypeptide of the invention or known fungal OXOX polypeptide by transforming a plant cell with an expression cassette that expresses a polynucleotide that inhibits the expression of the OXOX variant polypeptide or known fungal OXOX polypeptide. The polynucleotide may inhibit the expression of the OXOX variant polypeptide or known fungal OXOX polypeptide directly, by preventing transcription or translation of the OXOX variant or known fungal OXOX messenger RNA, or indirectly, by encoding a polypeptide that inhibits the transcription or translation of an OXOX gene modified to encode an OXOX variant polypeptide or known fungal OXOX polypeptide. Methods for inhibiting or eliminating the expression of a gene in a plant are well known in the art, and any such method may be used in the present invention to inhibit the expression of OXOX variant or known fungal OXOX polypeptide.

In accordance with the present invention, the expression of OXOX variant polypeptide or known fungal OXOX polypeptide is inhibited if the protein level of the OXOX variant polypeptide is less than 70% of the protein level of the same OXOX variant polypeptide or known fungal OXOX polypeptide in a plant that has not been genetically modified or mutagenized to inhibit the expression of that OXOX variant polypeptide or known fungal OXOX polypeptide. In particular embodiments of the invention, the protein level of the OXOX variant polypeptide or known fungal OXOX polypeptide in a modified plant according to the invention is less than 60%, less than 50%, less than 40%, less than 30%, less than 20%, less than 10%, less than 5%, or less than 2% of the protein level of the same OXOX variant polypeptide in a plant that has not been genetically modified to inhibit the expression of that OXOX variant polypeptide. The expression level of the OXOX variant polypeptide may be measured directly, for example, by assaying for the level of OXOX variant polypeptide expressed in the plant cell or plant, or indirectly, for example, by measuring the hydrogen peroxide produced by the OXOX variant polypeptide or known fungal OXOX polypeptide in the plant cell or plant, or by measuring the phenotypic changes in the plant. Methods for performing such assays are described elsewhere herein.

In other embodiments of the invention, the activity of the OXOX variant polypeptides or known fungal OXOX polypeptides is reduced or eliminated by transforming a plant cell with an expression cassette comprising a polynucleotide encoding a polypeptide that inhibits the activity of an OXOX variant polypeptide or known fungal OXOX polypeptide. The enhanced OXOX activity of an OXOX variant polypeptide or known fungal OXOX polypeptide is inhibited according to the present invention if the OXOX activity of the OXOX variant polypeptide or known fungal OXOX polypeptide is less than 70% of the OXOX activity of the same OXOX variant or known fungal OXOX polypeptide in a plant that has not been modified to inhibit the OXOX activity of that OXOX variant or known fungal OXOX polypeptide. In particular embodiments of the invention, the OXOX activity of the OXOX variant or known fungal OXOX polypeptide in a modified plant according to the invention is less than 60%, less than 50%, less than 40%, less than 30%, less than 20%, less than 10%, or less than 5% of the OXOX activity of the same OXOX variant or known fungal OXOX polypeptide in a plant that that has not been modified to inhibit the expression of that OXOX variant or known fungal OXOX polypeptide. The OXOX activity of an OXOX variant or known fungal OXOX polypeptide is “eliminated” according to the invention when it is not detectable by the assay methods described elsewhere herein. Methods of determining the alteration of OXOX activity of an OXOX variant or known fungal OXOX polypeptide are described elsewhere herein.

Thus, many methods may be used to reduce or eliminate the activity of an OXOX variant polypeptide or known fungal OXOX polypeptide. In addition, more than one method may be used to reduce the activity of a single OXOX variant polypeptide or known fungal OXOX polypeptide. In some embodiments of the present invention, a plant is transformed with an expression cassette that is capable of expressing a polynucleotide that inhibits the expression of an OXOX variant polypeptide of the invention or known fungal OXOX polypeptide. The term “expression” as used herein refers to the biosynthesis of a gene product, including the transcription and/or translation of said gene product. For example, for the purposes of the present invention, an expression cassette capable of expressing a polynucleotide that inhibits the expression of at least one OXOX variant or known fungal OXOX polypeptide is an expression cassette capable of producing an RNA molecule that inhibits the transcription and/or translation of at least one OXOX variant polypeptide of the invention or known fungal OXOX polypeptide. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Compositions of the invention comprise sequences encoding variants and fragments thereof. Methods of the invention involve the use of, but are not limited to, transgenic expression, antisense suppression, co-suppression, RNA interference, gene activation or suppression using transcription factors and/or repressors, mutagenesis including transposon tagging, directed and site-specific mutagenesis, chromosome engineering (see Nobrega et. al., Nature 431:988-993 (04)), homologous recombination, TILLING, and biosynthetic competition to manipulate, in plants and plant seeds and grains, the expression of seed proteins, including, but not limited to, those encoded by the sequences disclosed herein.

Other methods for decreasing or eliminating the expression of genes include the transgenic application of transcription factors (Pabo, C. O., et al. (2001) Annu Rev Biochem 70, 313-40.; and Reynolds, L., et al (2003), Proc Natl Acad Sci USA 100, 1615-20.), and homologous recombination methods for gene targeting (see U.S. Pat. No. 6,187,994).

Similarly, it is possible to eliminate the expression of a single gene by replacing its coding sequence with the coding sequence of a second gene using homologous recombination technologies (see Bolon, B. Basic Clin. Pharmacol. Toxicol. 95:4,12, 154-61 (2004); Matsuda and Alba, A., Methods Mol. Bio. 259:379-90 (2004); Forlino, et. al., J. Biol. Chem. 274:53, 37923-30 (1999)).

### Plant Genera

The OXOX variant or known fungal OXOX in combination with a pathogen tolerant background, as described in the present invention can be used over a broad range of plant types, including species from the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Picea, Caco, and Populus.

### Pathogens

As noted earlier, the OXOX variants of the invention or known fungal OXOX's can be utilized to protect plants from pathogens. Exemplary pathogens include but are not limited to fungi, bacteria, nematodes, viruses or viroids, parasitic weeds, pests include without limitation insects, biological agents, disease-producing microorganisms, toxic biological products, and organic biocides that can cause death or injury to humans, animals, and/or plants and the like. Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera. Insect pests of the invention for the major crops include: Maize: Ostrinia nubilalis, European corn borer; Agrotis ipsilon, black cutworm; Helicoverpa zea, corn earworm; Spodoptera frugiperda, fall armyworm; Diatraea grandiosella, southwestern corn borer; Elasmopalpus lignosellus, lesser cornstalk borer; Diatraea saccharalis, sugarcane borer; Diabrotica virgifera, western corn rootworm; Diabrotica longicornis barberi, northern corn rootworm; Diabrotica undecimpunctata howardi, southern corn rootworm; Melanotus spp., wireworms; Cyclocephala borealis, northern masked chafer (white grub); Cyclocephala immaculata, southern masked chafer (white grub); Popillia japonica, Japanese beetle; Chaetocnema pulicaria, corn leaf beetle; Sphenophorus maidis, maize billbug; Rhopalosiphum maidis, corn leaf aphid; Anuraphis maidiradicis, corn root aphid; Blissus leucopterus, chinch bug; Melanoplus femurrubrum, redlegged grasshopper; Melanoplus sanguinipes, migratory grasshopper, Hylemya platura, seedcorn maggot; Agromyza parvicornis, corn bloth leafminer; Anaphothrips obscurus, grass thrips; Solenopsis milesta, thief ant; Tetranychus urticae, twospotted spider mite; Sorghum: Chilo partellus, sorghum borer; Spodoptera frugiperda, fall armyworm; Helicoverpa zea, corn earworm; Elasmopalpus lignosellus, lesser cornstalk borer; Feltia subterranea, granulate cutworm; Phyllophaga crinita, white grub; Eleodes, Conoderus, and Aeolus spp., wireworms; Oulema melanopus, cereal leaf beetle; Chaetocnema pulicaria, corn flea beetle; Sphenophorus maidis, maize billbug; Rhopalosiphum maidis; corn leaf aphid; Sipha flava, yellow sugarcane aphid; Blissus leucopterus; chinch bug; Contarinia sorghicola, sorghum midge; Tetranychus cinnabarinus, carmine spider mite; Tetranychus urticae, twospotted spider mite; Wheat: Pseudaletia unipunctata, army worm; Spodoptera frugiperda, fall armyworm; Elasmopalpus lignosellus, lesser cornstalk borer; Agrotis orthogonia, pale western cutworm; Elasmopalpus lignosellus, lesser cornstalk borer; Oulema melanopus, cereal leaf beetle; Hypera punctata, clover leaf weevil; Diabrotica undecimpunctata howardi, southern corn rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctata, wheat bulb fly; Frankliniella fusca, tobacco thrips; Cephus cinctus, wheat stem sawfly; Aceria tulipae, wheat curl mite; Sunflower: Suleima helianthana, sunflower bud moth; Homoeosoma electellum, sunflower moth; Zygogramma exclamationis, sunflower beetle; Bothyrus gibbosus, carrot beetle; Neolasioptera murtfeldtiana, sunflower seed midge; Cotton: Heliothis virescens, cotton boll worm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentialis, differential grasshopper; Thrips tabaci, onion thrips; Frankliniella fusca, tobacco thrips; Tetranychus urticae, twospotted spider mite; Rice: Diatraea saccharalis, sugarcane borer; Spodoptera frugiperda, fall armyworm; Helicoverpa zea, corn earworm; Colaspis brunnea, grape colaspis; Lissorhoptrus oryzophilus, rice water weevil; Sitophilus oryzae, rice weevil; Nephotettix nigropictus, rice leafhopper; Blissus leucopterus, chinch bug; Acrosternum hilare, green stink bug; Soybean: Pseudoplusia includens, soybean looper; Anticarsia gemmatalis, velvetbean caterpillar; Plathypena scabra, green cloverworm; Ostrinia nubilalis, European corn borer; Agrotis ipsilon, black cutworm; Spodoptera exigua, beet armyworm; Heliothis virescens, cotton boll worm; Helicoverpa zea, cotton bollworm; Epilachna varivestis, Mexican bean beetle; Myzus persicae, green stink bug; Melanoplus femurrubrum, redlegged grasshopper; Melanoplus differentialis, differential grasshopper; Hylemya platura, seedcorn maggot; Sericothrips variabilis, soybean thrips; Thrips tabaci, onion thrips; Tetranychus turkestani, strawberry spider mite; Tetranychus urticae, twospotted spider mite; Barley: Ostrinia nubilalis, European corn borer; Agrotis ipsilon, black cutworm; Schizaphis graminum, greenbug; Blissus leucopterus leucopterus, chinch bug; Acrosternum hilare, green stink bug; Euschistus servus, brown stink bug; Jylemya platura, seedcorn maggot; Mayetiola destructor, Hessian fly; Petrobia latens, brown seedcorn maggot; Mayetiola destructor, Hessian fly; Petrobia latens, brown seedcorn maggot; Mayetiola destructor, Hessian fly; Petrobia latens, brown wheat mite; Oil Seed Rape: Vrevicoryne brassicae, cabbage aphid.

Generally viruses include tobacco or cucumber mosaic virus, ringspot virus, necrosis virus, maize dwarf mosaic virus, etc. specific viral, fungal and bacterial pathogens for the major crops include: Soybeans: Phytophthora megasperma fsp. Glycinea, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotrichum truncatum), Corynespora cassiicola, Septoria glycines, Phyllosticta sojicola, Alternaria alternata, Pseudomonas syringae p.v. glycinea, Xanthomonas campestris p.v. phaseoli, Microsphaera diffusa, Fusarium semitectum, Phialophora gregata, Soybean mosaic virus, Glomerella glycines, Tobacco Ring spot virus, Tobacco Streak virus, Phakopsora pachyrhizi, Pythium aphanidermatum, Pythium ultimum, Pythium debaryanum, Tomato spotted wilt virus, Heterodera glycines Fusarium solani; Canola. Albugo candida, Alternaria brassicae, Leptosphaeria maculans, Rhizoctonia solani, Sclerotinia sclerotiorum, Mycosphaerella brassiccola, Pythium ultimum, Peronospora parasitica, Fusarium roseum, Alternaria alternata; Alfalfa: Clavibater michiganensis subsp. Insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium oxysporum, Rhizoctonia solani, Uromyces striatus, Colletotrichum trifolii race 1 and race 2, Leptosphaerulina briosiana, Stemphylium botryosum, Stagonospora meliloti, Sclerotinia trifoliorum, Alfalfa Mosaic Virus, Verticillium albo-atrum, Xanthomonas campestris p.v. alfalfae, Aphanomyces euteiches, Stemphylium herbarum, Stemphylium alfalfae; Wheat: Pseudomonas syringae p.v. atrofaciens, Urocystis agropyri, Xanthomonas campestris p.v. translucens, Pseudomonas syringae p.v. syringae, Alternaria alternata, Cladosporium herbarum, Fusarium graminearum, Fusarium avenaceum, Fusarium culmorum, Ustilago tritici, Ascochyta tritici, Cephalosporium gramineum, Colletotrichum graminicola, Erysiphe graminis f.sp. tritici, Puccinia graminis f.sp. tritici, Puccinia recondita f.sp. tritici, Puccinia striiformis, Pyrenophora tritici-repentis, Septoria nodorum, Septoria tritici, Septoria avenae, Pseudocercosporella herptotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Barley Yellow Dwarf Virus, Brome Mosaic Virus, Soil Borne Wheat Mosaic Virus, Wheat Streak Mosaic Virus, Wheat Spindle Streak Virus, American Wheat Striate Virus, Claviceps purpurea, Tilletia tritici, Tilletia laevis, Ustilago tritici, Tilletia indica, Rhizoctonia solani, Pythium arrhenomanes, Pythium graminicola, Pythium aphanidermatum, High Plains Virus, European wheat striate virus; Sunflower: Plasmophora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahlia, Erwinia carotovora pv. carotovora, Cephalosporium acremonium, Phytophthora cryptogea, Albugo tragopogonis; Maize: Fusarium moniliforme var. subglutinans, Erwinia stewartii, Fusarium moniliforme, Gibberella zeae (Fusarium graminearum), Stenocarpella maydis (Diplodia maydis), Pythium irregulare, pythium debaryanum, Pythium graminicola, Pythium splendens, Pythium ultimum, Pythium aphanidermatum, Aspergillus flavus, Bipolaris maydis O, T Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella zea, Colletotrichum graminicola, Cercospora zeae-maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Maize Dwarf Mosaic Virus A & B, Wheat Streak Mosaic Virus, Maize Chlorotic Dwarf Virus, Claviceps sorghi, Pseudonomas avenae, Erwinia chrysanthemi pv. Zea, Erwinia carotovora, Corn stunt spiroplasma, Diplodia macrospora, Sclerophthora macrospora, Peronosclerospora sorghi, Peronosclerospora philippinensis, Peronosclerospora maydis, Peronosclerospora sacchari, Spacelotheca reiliana, Physopella zeae, Cephalosporium maydis, Cephalosporium acremonium, Maize chlorotic mottle virus, High plains virus, Maize mosaic virus, Maize rayado fino virus, Maize streak virus, Maize stripe virus, Maize rough dwarf virus; Sorghum: Exserohilum turcicum, Colletotrichum graminicola (Glomerella graminicola), Cercospora sorghi, Gloeocercospora sorghi, Ascochyta sorghi, Pseudomonas syringae p.v. syringae, Xanthomonas campestris p. v. holcicola, Pseudomonas andropogonis, Puccinia purpurea, Macrophomina phaseolina, Periconia circinata, Fusarium moniliforme, Alternaria alternate, Bipolaris sorghicola, Helminthosporium sorghicola, Curvularia lunata, Phoma insidiosa, Pseudomonas avenae (Pseudomonas alboprecipitans), Ramulispora sorghi, Ramulispora sorghicola, Phyllachara sacchari, Sporisorium relianum (Sphacelotheca reliana), Sphacelotheca cruenta, Sporisorium sorghi, Sugarcane mosaic H, Maize Dwarf Mosaic Virus A & B, Claviceps sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium Oxysporum, Pythium arrhenomanes, Pythium graminicola, etc.

Generally parasitic weeds include the parasitic flowering plants Orobanche spp. (Broomrape), the mistletoes (Lorranthaceae: genera Arceuthobrium, Viscum, and Phoradendron, dodder (Cuscuta spp.), and Striga spp. (Witchweeds). Parasitic weeds of the present invention include, but are not limited to, Sunflower and Canola: Orobanche aegyptiaca, Orabanche cumana, Tomato and Potato: Orobanche aegyptiaca, Orobanche ramosa, Orobanche cernua, etc.

### Gene Transformation Methods

Numerous methods for introducing foreign genes into plants are known and can be used to insert a gene into a plant host, including biological and physical plant transformation protocols. See, for example, Miki et al., (1993) “Procedure for Introducing Foreign DNA into Plants”, In: Methods in Plant Molecular Biology and Biotechnology, Glick and Thompson, eds., CRC Press, Inc., Boca Raton, pages 67-88. The methods chosen vary with the host plant, and include chemical transfection methods such as calcium phosphate, microorganism-mediated gene transfer such as Agrobacterium (Horsch, et al., Science, 227: 1229-31 (1985)), electroporation, micro-injection, and biolistic bombardment. See also WO200037663. herein incorporated by reference.

Expression cassettes and vectors and in vitro culture methods for plant cell or tissue transformation and regeneration of plants are known and available. See, for example, Gruber, et al., (1993) “Vectors for Plant Transformation” In: Methods in Plant Molecular Biology and Biotechnology, Glick and Thompson, eds. CRC Press, Inc., Boca Raton, pages 89-119.

### Agrobacterium-Mediated Transformation

The most widely utilized method for introducing an expression vector into plants is based on the natural transformation system of Agrobacterium. A. tumefaciens and A. rhizogenes are plant pathogenic soil bacteria which genetically transform plant cells. The Ti and Ri plasmids of A. tumefaciens and A. rhizogenes, respectfully, carry genes responsible for genetic transformation of plants. See, for example, Kado, Crit. Rev. Plant Sci., 10:1-32 (1991). Descriptions of the Agrobacterium vector systems and methods for Agrobacterium-mediated gene transfer are provided in Gruber et al., supra; and Moloney, et al., Plant Cell Reports, 8: 238-242 (1989).

### Direct Gene Transfer

Despite the fact that the host range for Agrobacterium-mediated transformation is broad, some major cereal crop species and gymnosperms have generally been recalcitrant to this mode of gene transfer, even though some success has recently been achieved in rice (Hiei et al., The Plant Journal, 6: 271-282 (1994)) and maize (Ishida, et al., Nature Biotech., 14: 754-750 (1996)). Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes. (Sanford, et al., Part. Sci. Technol., 5: 27-37 (1987); Sanford, Trends Biotech, 6: 299-302 (1988); Sanford, Physiol. Plant, 79: 206-209 (1990); Klein, et al., Biotechnology, 10: 286-291 (1992)).

Another method for physical delivery of DNA to plants is sonication of target cells as described in Zang, et al., BioTechnology, 9: 996-996 (1991). Alternatively, liposome or spheroplast fusions have been used to introduce expression vectors into plants. See, for example, Deshayes, et al., EMBO J., 4: 2731-2737 (1985); and Christou, et al., Proc. Nat'l Acad. Sci. (USA), 84: 3962-3966 (1987). Direct uptake of DNA into protoplasts using CaCl₂ precipitation, polyvinyl alcohol or poly-L-ornithine have also been reported. See, for example, Hain, et al., Mol. Gen. Genet., 199: 161 (1985); and Draper, et al., Plant Cell Physiol., 23: 451-458 (1982).

Electroporation of protoplasts and whole cells and tissues has also been described. See, for example, Donn, et al., (1990) In: Abstracts of the VIIth Int; 1 Congress on Plant Cell and Tissue Culture IAPTC, A2-38, page 53; D'Halluin et al., Plant Cell, 4: 1495-1505 (1992); and Spencer et al., Plant Mol. Biol., 24: 51-61 (1994).

### Particle Wounding/Agrobacterium Delivery

Another useful basic transformation protocol involves a combination of wounding by particle bombardment, followed by use of Agrobacterium for DNA delivery, as described by Bidney, et al., Plant Mol. Biol., 18: 301-31 (1992). Useful plasmids for plant transformation include Bin 19. See Bevan, Nucleic Acids Research, 12: 8711-8721 (1984), and hereby incorporated by reference. This method is preferred for transformation of sunflower plants.

In general, the intact meristem transformation method involves imbibing seed for 24 hours in the dark, removing the cotyledons and root radical, followed by culturing of the meristem explants. Twenty-four hours later, the primary leaves are removed to expose the apical meristem. The explants are placed apical dome side up and bombarded, e.g., twice with particles, followed by co-cultivation with Agrobacterium. To start the co-cultivation for intact meristems, Agrobacterium is placed on the meristem. After about a 3-day co-cultivation period the meristems are transferred to culture medium with cefotaxime plus kanamycin for the NPTII selection.

The split meristem method involves imbibing seed, breaking of the cotyledons to produce a clean fracture at the plane of the embryonic axis, excising the root tip and then bisecting the explants longitudinally between the primordial leaves. The two halves are placed cut surface up on the medium then bombarded twice with particles, followed by co-cultivation with Agrobacterium. For split meristems, after bombardment, the meristems are placed in an Agrobacterium suspension for 30 minutes. They are then removed from the suspension onto solid culture medium for three day co-cultivation. After this period, the meristems are transferred to fresh medium with cefotaxime plus kanamycin for selection.

### Transfer by Plant Breeding

Alternatively, once a single transformed plant has been obtained by the foregoing recombinant DNA method, conventional plant breeding methods can be used to transfer the gene and associated regulatory sequences via crossing and backcrossing. Such intermediate methods will comprise the further steps of (1) sexually crossing the disease-resistant plant with a plant from the disease susceptible taxon; (2) recovering reproductive material from the progeny of the cross; and (3) growing disease-resistant plants from the reproductive material. Where desirable or necessary, the agronomic characteristics of the susceptible taxon can be substantially preserved by expanding this method to include the further steps of repetitively: (1) backcrossing the disease-resistant progeny with disease-susceptible plants from the susceptible taxon; and (2) selecting for expression of a hydrogen peroxide producing enzyme activity (or an associated marker gene) among the progeny of the backcross, until the desired percentage of the characteristics of the susceptible taxon are present in the progeny along with the gene or genes imparting oxalic acid degrading and/or hydrogen peroxide enzyme activity.

By the term “taxon” herein is meant a unit of botanical classification. It thus includes, genus, species, cultivars, varieties, variants and other minor taxonomic groups which lack a consistent nomenclature.

### Sclerotinia Disease

Sclerotinia overwinters as dense, black hyphal masses (sclerotia) deposited in the soil. Sclerotia in the soil germinate when favorable conditions are present to produce mycelial growth for root infections or apothecia for above ground ascospore production. Sclerotinia infection in sunflower manifests itself in 4 basic forms; basal root mycelial infection leading to wilt, and middle stalk, bud and head rots. Airborne ascospores from soil surface apothecia are responsible for the later three infections. The general view has been that Sclerotinia does not invade healthy tissue but gains a foothold only in wounded areas or senescing tissue where the spores happen to land. This does not appear to be strictly true, however, in that the only correlation to be made for successful ascospore infection in plants is the number of hours of continuous moisture to which spores are exposed during the germination process. Anywhere from 24 to 48 hours of damp conditions as well as some minimal level of plant exudate as a nutritional source are required for spore germination and penetration.

Fungal produced oxalate, in conjunction with a host of degradative enzymes, appears to be a requirement for infection (Noyes, R. D. and J. G. Hancock, Physiol. Plant Pathol., 18(2): 123-132 (1981)). Mutant strains of Sclerotinia deficient in oxalate production are no longer pathogenic even though other degradative enzymes are produced (Godoy, G., et al., Physiol. Mol. Plant. Pathol., 37(3): 179-191 (1990). In addition, oxalate fed to sunflower plants exhibit the wilt symptoms of Sclerotinia infection. Therefore, oxalate acts as a classic, diffusable toxin by stressing host plant tissue in preparation for enzymatic degradation and mycelial colonization (Maxwell, D. P., Physiol. Plant Pathol., 3(2): 279-288 (1973)).

### Tolerant Backgrounds

In one aspect, an OXOX of the present invention or known fungal OXOX may be expressed in a plant having a pathogen tolerant genetic background. Without wishing to be bound by this theory, it is believed that the combination of expression of an OXOX of the present invention or known fungal OXOX in the pathogen tolerant genetic background would act syngeristically to confer increased disease resistance compared to the expression of the OXOX in a non-tolerant background and potentially produce an immune or near immune plant.

Introduction of an OXOX into a Tolerant Background

One way of introducing an OXOX variant or known fungal OXOX is by transforming a non-tolerant plant with an expression vector containing the enzyme and regenerating plants. Next the transgenic plants expressing the enzyme are crossed with a plant tolerant to the pathogen. Alternatively, a tolerant plant or plant tissue could be transformed with the expression vector containing the enzyme. The resulting plant would contain both a transgene expressing the enzyme and a genetically tolerant background.

Another method could be overexpression of an endogenous mutated OXOX gene. In some embodiments, isolated nucleic acids that serve as promoter or enhancer elements can be introduced in the appropriate position (generally upstream) of the mutated gene(s) encoding an enzyme of the present invention so as to up or down regulate expression of that enzyme. For example, endogenous promoters can be altered in vivo by mutation, deletion, and/or substitution (see: Kmiec, U.S. Pat. No. 5,565,350; Zarling et al., PCT/US93/03868), or isolated promoters can be introduced into a plant cell in the proper orientation and distance from a mutated OXOX gene so as to control the expression of the gene. Gene expression can be modulated under conditions suitable for plant growth so as to alter the enzyme content and/or composition. Thus, the present invention provides compositions, and methods for making, exogenous promoters and/or enhancers operably linked to a mutated, endogenous form of an enzyme of the present invention.

This invention can be better understood by reference to the following non-limiting examples. It will be appreciated by those skilled in the art that other embodiments of the invention may be practiced without departing from the spirit and the scope of the invention as herein disclosed and claimed.

### Example 1

### Cloning of Fungal OXOX

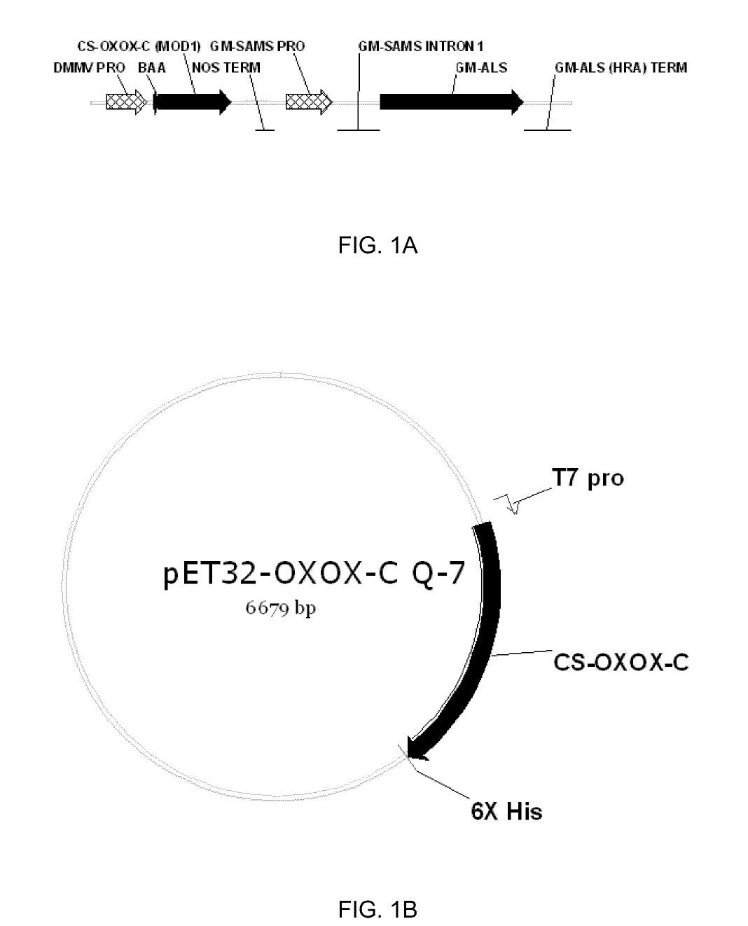

Sequences of both OXOX C and G alleles were published by Escutia et al., 2005 and were deposited with EMBL: OXOX-C partial genomic DNA, Acession No. AJ746414; OXOX-C partial cDNA, Acession No. AJ563659; OXOX-G genomic DNA, Acession No. AJ563660; OXOX-G cDNA, Acession No. AJ746412. See also, Escutia et al., Cloning and sequencing of two Ceriporiopsis subvermispora bicupin oxalate oxidase allelic isoforms: implications for the reaction specificity of oxalate oxidases and decarboxylases. (2005). Based on published sequence, mature proteins of OXOX C (SEQ ID NO: 21) and G (SEQ ID NO: 22) were synthesized and fused to N-terminal barley alpha amylase signal sequence (BAA ss; SEQ ID NO:3). The fungal OXOX coding sequence was synthesized with codon usage suitable for expression in soybean (FIG. 1A) and E. coli (FIG. 1B).

### Example 2

### E. coli Expression of OXOX

The E. coli expression system was based on the published protocol of Escutia et al. Escutia et al., Cloning and sequencing of two Ceriporiopsis subvermispora bicupin oxalate oxidase allelic isoforms: implications for the reaction specificity of oxalate oxidases and decarboxylases. (2005). The coding sequence for the mature OXOX enzyme was inserted in E. coli expression vector pET32 (Invitrogen) to include a 6× histidine tag at the C-terminus. The resulting expression plasmid was transformed into E. coli strain BL21Star pLysS (Invitrogen).

E. coli cultures were grown at 37° C. At optical density 0.4, arabinose was added to a concentration or 0.4%. After another hour of growth at 37° C., MnCl2 was added to 5 mM and IPTG to 1 mM. Cultures were then grown at 25° C. for 16 hours. Cells were harvested by centrifugation at 4000 rpm for 10 min. The supernatant was discarded and cell pellets were kept at −80° C. for at least one hour. Pellets were resuspended in total 40 ml of lysis buffer with 50 mM phosphate buffer pH7, 2 mg/ml protease inhibitor (sigma P-8465), 100 mM KCL, 1/1000 lysozyme, 1/2000 endonuclease, and incubated at 37° C. with shaking for one hour, followed by sonication. Cellular debris was removed by centrifugation and the clarified supernatant was poured into a new tube. Nickel resin (Qiagen) was added. After a short period of incubation, the slurry was poured onto a column and washed with buffer. Purified OXOX protein was eluted from the resin in 50 mM succinate pH 5, 100 mM KCl. The E. coli expression procedure was scalable allowing large scale protein production for kinetic analysis as well as small scale production for high throughput screening of libraries.

### Example 3

### OXOX Enzymatic Activity Determination

Oxalate oxidase enzymatic activity was determined in a coupled reaction. Oxalate oxidase converts oxalic acid to carbon dioxide and hydrogen peroxide. In the presence of horse radish peroxidase, hydrogen peroxide reacts with 3-methyl-2-benzothiazolinone hydrazone (MBTH) and N,N-dimethylaniline (DMA) to form indamine dye, which can be detected spectrophotmetrically or colorimetrically as described by Laker, M. F., Hoffman, A. F., and Meeuse, J. D. (1980) Clinical Chemistry 26, 827-830. The coupled reaction was used for characterization of oxalate oxidase kinetic properties as well as in a screening procedure to identify oxalate oxidase variants with improved enzymatic properties.

A quick OXOX assay was developed to identify OXOX positive transgenic plants and quantify OXOX activity in transgenic plants as previously described with modifications (Hu et, al, 2005). A single leaf disk was harvested into 96-well plate from an individual plant. Lyophilized leaf powder or fresh leaf disk was suspended or extracted in 100 mM sodium succinate (pH 3.5). The reaction was started by adding oxalic acid to a final concentration of 1 mM, incubating at 37° C. for 5 min. An aliquot of the extract supernatant from each sample was mixed with an H₂O₂-detecting reagent containing 200 mM Tris (pH 7.0), 400 μM of 4-aminoantipyrine, 20 μL of N,N-dimethylanaline and 2 units of horseradish peroxidase (Sigma, St. Louis). Expression level of OXOX was measured as the absorbance (O.D.) reading at wavelength 550 nm on the plate reader.

### Example 4

### Removal of Glycosylation Sites of a Fungal OXOX

Three potential glycosylations sites with the consensus amino acid sequence N—X—(S/T) were identified in OXOX-C at amino acid positions 60, 384 and 430 in SEQ ID NO:27. Site-directed mutagenesis was used to alter these sites (Quickchange, Stratagene). Every possible amino acid substitution was generated at the third amino acid position of each potential glyosylation site. The resulting variants were screened for activity using the enzymatic assay described in Example 3. After two rounds of screening and recombining useful mutations, OXOX-C variants were identified that had all three glycosylation sites mutated and which retained activity comparable to wild-type OXOX-C. These variants correspond to sequences OXOX-C-MOD1-ALT1 (SEQ ID NO:23) and OXOX-C-MOD1-ALT2 (SEQ ID NO:24).

### Example 5

### Shuffling of Fungal OXOX

DNA shuffling was performed as described elsewhere herein. A polymerase chain reaction product corresponding to the OXOX-C coding sequence was fragmented by limited nuclease treatment. Synthetic oligonucleotides encoding sequence diversity from oxalate decarboxylase sequences found in public databases were added. A polymerase chain reaction procedure was performed on the mixture to yield a library of full length OXOX-C coding sequences with additional diversity incorporated. The resulting library was inserted in expression vector pET32, E. coli cells were transformed and protein was expressed, as described in Example 2. A tiered screening strategy was devised based on the OXOX protein expression and enzymatic activity determination procedures described in Examples 2 and 3, respectively. A high throughput screening process based on colorimetric determination of OXOX activity was utilized to identify individual E. coli strains harboring active OXOX variants. These OXOX-active strains were reassayed using quantitative spectrophotometric determination of OXOX activity. Finally, purified OXOX protein was produced for the most active shuffled variants and subjected to detailed kinetic analysis. Three rounds of this iterative screening and selection procedure were completed, and OXOX variants with up to 8-fold improved activity were identified

The kinetic parameters of various OXOX variants at pH 3.8, pH4.8 and pH5.8 are shown in Table 5. Iterative rounds of gene shuffling resulted in OXOX variants with decreased Km and/or increased Kcat relative to the polypeptide corresponding to sequence ALT1 (WT-Q7) at pH5.8. Two classes of improved enzymes emerged: those with dramatically lowered Km, exemplified by variants 3-21 (ALT6) and 3-20, and those with dramatically improved Kcat, exemplified by variants 3-25 (ALT7) and 3-26. More modest improvements in Km and Kcat were observed under lower pH conditions. Coupled reaction was preformed as described in Example 3. Enzyme kinetic parameters were determined by standard methods as described in Segel (1976) Biochemical calculations, 2nd Edition. John Wiley & Sons, London, New York, Sydney, Toronto.

TABLE 5
Kinetic parameters of OXOX variants.

| | pH 3.8 | | pH 4.8 | | pH 5.8 | |
|---|---|---|---|---|---|---|
| | Kcat | Km | Kcat | Km | Kcat | Km |
| | (/s) | (mM) | (/s) | (mM) | (/s) | (mM) |
| OXOX-C | 5.45 | 0.35 | 2.74 | 1.24 | 0.91 | 5.95 |
| (SEQ IS NO: 18) | | | | | | |
| OXOX-G | 4.80 | 0.40 | 2.56 | 1.37 | 0.82 | 6.14 |
| (SEQ IS NO: 19) | | | | | | |
| ALT1 (WT-Q7) | 5.00 | 0.33 | 2.70 | 1.21 | 0.85 | 6.01 |
| (SEQ IS NO: 23) | | | | | | |
| ALT2 (glyc-) | 5.60 | 0.28 | 2.99 | 1.24 | 0.91 | 5.9 |
| (SEQ IS NO: 24) | | | | | | |
| ALT3 (1-8) | 4.69 | 0.31 | 3.21 | 1.70 | 1.36 | 8.38 |
| (SEQ IS NO: 25) | | | | | | |
| ALT4 (1-23) | 4.08 | 0.35 | 2.70 | 1.32 | 0.85 | 5.51 |
| (SEQ IS NO: 26) | | | | | | |
| ALT5 (2-6) | 10.01 | 0.46 | 7.06 | 2.32 | 2.44 | 8.91 |
| (SEQ IS NO: 27) | | | | | | |
| ALT6 (3-21) | 2.79 | 0.12 | 2.61 | 0.55 | 1.72 | 1.46 |
| (SEQ IS NO: 28) | | | | | | |
| ALT7 (3-25) | 12.01 | 0.45 | 8.28 | 2.17 | 5.85 | 7.84 |
| (SEQ IS NO: 29) | | | | | | |
| FG10 | 8.01 | 0.40 | 6.00 | 1.83 | 2.65 | 5.9 |
| (SEQ IS NO: 30) | | | | | | |
| FG23 | 7.06 | 0.38 | 4.62 | 1.21 | 2.61 | 5.25 |
| (SEQ IS NO: 31) | | | | | | |
| 3-10 | 11.44 | 0.52 | 5.58 | 1.74 | 2.61 | 6.21 |
| (SEQ IS NO: 32) | | | | | | |
| 3-20 | 3.64 | 0.15 | 3.81 | 0.78 | 2.35 | 2.32 |
| (SEQ IS NO: 33) | | | | | | |
| 3-26 | 11.44 | 0.48 | 6.67 | 1.78 | 5.22 | 7.74 |
| (SEQ IS NO: 34) | | | | | | |
| 183-FG15 | 9 | 0.26 | — | — | 4.3 | 2.0 |
| (SEQ ID NO: 100) | | | | | | |
| 5-4G | 17.7 | 0.28 | — | — | 6.3 | 4.3 |
| (SEQ ID NO: 101) | | | | | | |
| 5-7G | 21 | 0.31 | — | — | 7.2 | 5.0 |
| (SEQ ID NO: 102) | | | | | | |
| 5-8G | 19.8 | 0.26 | — | — | 7.3 | 6.4 |
| (SEQ ID NO: 103) | | | | | | |
| FG-B5 | 13.7 | 0.34 | — | — | 7.1 | 3.7 |
| (SEQ ID NO: 104) | | | | | | |
| FG-E5 | 21.1 | 0.37 | — | — | 11.9 | 5.3 |
| (SEQ ID NO: 105) | | | | | | |
| FG-G6 | 17.2 | 0.31 | — | — | 12.6 | 6.9 |
| (SEQ ID NO: 106) | | | | | | |

### Example 6

### Soybean Transformation

Polynucleotides of SEQ ID NO:4-12 were used for soybean transformation and the generation of transgenic soybean plants using the methods described below.

Soybean embryos were bombarded with a plasmid containing the OXOX sequence operably linked to the Mirabilis Mosaic Caulimovirus (dMMV) promoter with double enhancer domain. To induce somatic embryos, cotyledons, 3-5 mm in length were dissected from surface-sterilized, immature seeds of the soybean cultivar Jack or 93B86, are cultured in the light or dark at 26° C. on an appropriate agar medium for six to ten weeks. Somatic embryos producing secondary embryos were then excised and placed into a suitable liquid medium. After repeated selection for clusters of somatic embryos that multiplied as early, globular-staged embryos, the suspensions were maintained as described below.

Soybean embryogenic suspension cultures were maintained in 35 ml liquid media on a rotary shaker, 150 rpm, at 26° C. with florescent lights on a 16:8 hour day/night schedule. Cultures were subcultured every two weeks by inoculating approximately 35 mg of tissue into 35 ml of liquid medium.

Soybean embryogenic suspension cultures were be transformed by the method of particle gun bombardment (Klein et al. (1987) Nature (London) 327:70-73, U.S. Pat. No. 5,955,050). A DuPont Biolistic PDS1000/HE instrument (helium retrofit) was for these transformations.

A selectable marker gene that was used to facilitate soybean transformation is a transgene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188), and the 3′ region of the nopaline synthase gene from the T-DNA of the Ti plasmid of Agrobacterium tumefaciens. The expression cassette comprising the OXOX operably linked to the dMMV promoter was isolated as a restriction fragment. This fragment was then inserted into a unique restriction site of the vector carrying the marker gene.

To 50 μl of a 60 mg/ml 1 μm gold particle suspension was added (in order): 5 μl DNA (1 μg/μl), 20 μl spermidine (0.1 M), and 50 μl CaCl₂ (2.5 M). The particle preparation was then agitated for three minutes, spun in a microfuge for 10 seconds and the supernatant removed. The DNA-coated particles were then washed once in 500 μA 70% ethanol and resuspended in 50 μA of anhydrous ethanol. The DNA/particle suspension were sonicated three times for one second each. Five microliters of the DNA-coated gold particles were then loaded on each macro carrier disk.

Approximately 300-500 mg of a two-week-old suspension culture was placed in an empty 60×15 mm petri dish and the residual liquid removed from the tissue with a pipette. For each transformation experiment, approximately 5-10 plates of tissue are normally bombarded. Membrane rupture pressure was set at 1100 psi, and the chamber was evacuated to a vacuum of 28 inches mercury. The tissue was placed approximately 3.5 inches away from the retaining screen and bombarded three times. Following bombardment, the tissue was divided in half and placed back into liquid and cultured as described above.

Five to seven days post bombardment; the liquid media was exchanged with fresh media, and eleven to twelve days post-bombardment with fresh media containing 50 mg/ml hygromycin. This selective media was refreshed weekly. Seven to eight weeks post-bombardment, green, transformed tissue was observed growing from untransformed, necrotic embryogenic clusters. Isolated green tissue was removed and inoculated into individual flasks to generate new, clonally propagated, transformed embryogenic suspension cultures. Each new line was treated as an independent transformation event. These suspensions were then subcultured and maintained as clusters of immature embryos or regenerated into whole plants (TO) by maturation and germination of individual somatic embryos. T0 soybean plants were transferred into soil to generate T1 seeds. In addition, T0 plants were also used for both expression study and leaf disk bioassay against Sclerotinia as described in Example 7.

### Example 7

### Testing of OXOX Variants

T0 transgenic plants expressing OXOX-C-ALT1 (SEQ ID NO:23) were selected to carry out leaf disk assay against Sclerotinia pathogen. Six uniform T0 leaves were harvested from T0 plants and placed in a 100×20 mm sterile Petri plate with 7.5 cm wet filter paper. For plug inoculation, a 6 mm plug of fungal culture was cut from 1/32-strength PDA (1.5% and 0.4% Bacto agar) and placed on top of T0 leaf. For petiole inoculations, 10 μl pipette tips were filled with the same fungal culture and place the tips over the cut petiole of the middle leaflet. Petri dishes were transferred in plastic containers and placed in the growth chamber at 23° C. with 70% RH in the dark. Disease lesions areas (mm²) for plug inoculation and lesion distance (mm) for petiole inoculation were recorded at 48, 72, and 96 hr post inoculation.

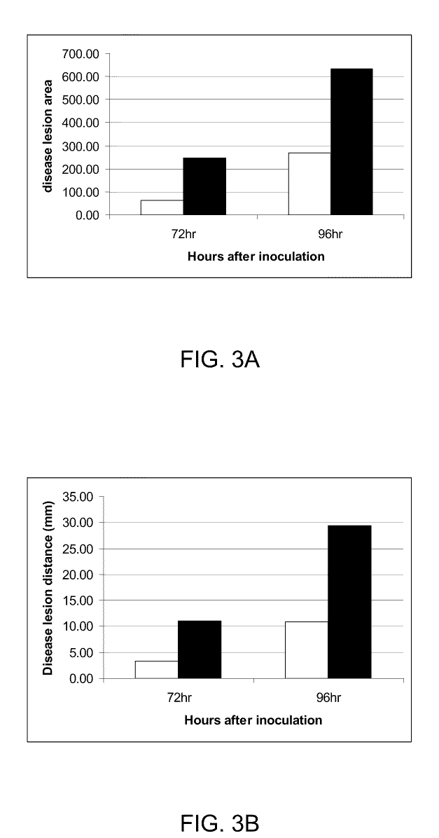

OXOX expression of individual OXOX and control plants were measured using a single leaf disk as described in Example 3. Average disease scores were compared in FIGS. 3A and 3B from 17 OXOX positive events and 17 negative events. T0 leaves with OXOX expression showed significant smaller lesions. These eight transgenic events (5853.1.6, 5853.2.2, 5853.3.5, 5805.1.9, 5805.2.2, 5805.3.6, 5805.4.16, 5805.4.9) and two transgenic control lines (4626.7.3 and 4626.7.4) were further tested in growth chamber at T1 generation as describe in Example 8.

### Example 8

### Growth Chamber Assay Against Sclerotinia

Five T1 transgenic soybean seeds were selected and planted 1-inch deep in a sterile, 4-inch pot filled with potting soil. After emergence the seedlings were thinned to three. Four-week-old plants were inoculated using a modified straw inoculation method (Boland, et al., 2004) as follows. 20 μl of pipette tip with plug was used to bore into the leading edge of a growing culture of S. sclerotiorum. The petiole of the third trifoliate was cut 1 inch from the stem and a pipette tip loaded with a fungal plug was placed over the cut petiole. Inoculated plants were placed in the growth chamber for 16-20 hours in dark at 74° F.

Experiments were scored 5-7 days following inoculation. Plants were evaluated individually on a 1-9 rating scale, where: 9=No symptom or small necrotic lesion on the main stem, where the inoculated petiole is attached; 7=Restricted fungal growth, lesion on the main stem <1″ in length; 5=Lesion >1″ in length, plant has no sign of wilting; 3=Plant starts to wilt or partially wilt, branches remain healthy; 1=Main stem wilting all the way to the tip (growing point), whole plant wilting and dying. Each experiment was consisted of 6 replications (pots) with 3 plants (subsamples) per pot.

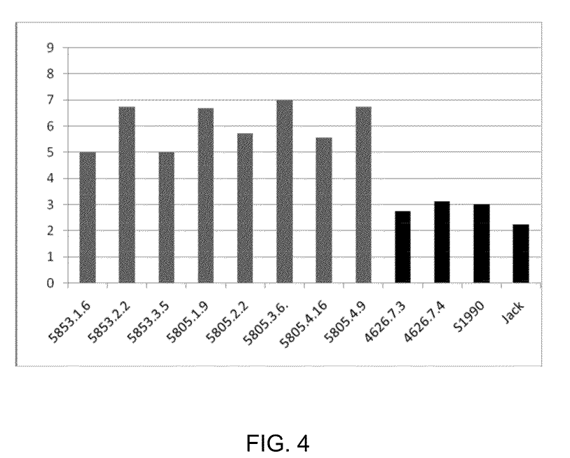

Eight OXOX positive events (OXOX-C-ALT1) showed significant improved resistance against Sclerotinia infection compared with Jack and transgenic Jack controls (expressing ALS marker only). All OXOX positive plants were also recorded 2 scores better than the most tolerant commercial cultivar S1990 (FIG. 4).

### Example 9

### Synthetic Gastric Fluid Assay

A synthetic gastric fluid assay may be performed as an indicator of the stability of a protein in the mammalian gut. Stability in the gut can affect the potential allergenicity of a protein. The assay conditions mimic those found in the gut of a mammal. Specifically, 200 milligrams of sodium chloride are dissolved in 100 milliliters of water. The pH is adjusted to 1.2 by addition of hydrochloric acid. The gastric protease pepsin is added such that there are 10 units of pepsin per microgram of oxalate oxidase. The assay temperature is 37° C. One hundred microliters of oxalate oxidase protein at 5 milligrams per milliliter are added to 1.9 milliliter of the assay mixture. One hundred twenty microliter aliquots are removed from the reaction at various timepoints, for example 0, 0.5, 1, 2, 5, 10, 20, 30, 60 minutes, and added to forty-eight microliters of two hundred millimolar sodium carbonate (Na₂CO₃) to stop the reaction. Samples from each timepoint are subjected to SDS-polyacrylamide gel electrophoresis. The amount of oxalate oxidase protein remaining at each timepoint is estimated by the band intensity after Coomassie staining of the gel. If an oxalate oxidase variant is less stable in synthetic gastric fluid, less material will be present at the various timepoints.

A shuffled oxalic acid variant, such as the polypeptides encoded by SEQ ID NO:8, SEQ ID NO:11 and SEQ ID NO:12, subjected to these assay conditions may show decreased stability compared with the other oxalate oxidase proteins, e.g those encoded by SEQ ID NO:1, SEQ ID NO:2 and SEQ ID NO:6, as seen in FIG. 5. This may be a beneficial property because lower stability in the gastric fluid may indicate a reduction in any potential allergenicity. (Fu et al. J Agric Food Chem. 2002 Nov. 20; 50(24):7154-60. Digestibility of food allergens and nonallergenic proteins in simulated gastric fluid and simulated intestinal fluid-a comparative study; Astwood et al. Nat. Biotechnol. 1996 October; 14(10):1269-73. Stability of food allergens to digestion in vitro.)

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. An isolated or recombinant nucleic acid comprising an oxalate oxidase (OXOX) variant polynucleotide comprising a member selected from the group consisting of:
(a) an isolated OXOX variant polynucleotide comprising a nucleotide that has been substituted and wherein the nucleotide substitution is one or more of the substitutions shown in FIG. 5;
(b) an isolated OXOX variant polynucleotide encoding a polypeptide comprising an amino acid sequence that has been substituted and wherein the amino acid substitution is least one amino acid substitution at a position that corresponds to position 10, 19, 23, 26, 29, 35, 36, 38, 39, 40, 53, 54, 57, 58, 60, 61, 62, 63, 65, 68, 72, 79, 81, 83, 99, 102, 107, 115, 118, 124, 127, 131, 144, 148, 154, 159, 164, 166, 171, 174, 177, 181, 190, 192, 196, 200, 202, 203, 218, 219, 245, 259, 269, 278, 282, 287, 289, 290, 339, 349, 353, 359, 363, 373, 384, 387, 394, 395, 396, 399, 410, 425, 426, 427, 430, 433 or 436 of SEQ ID NO: 37 or an additional amino acid residue at position 437 or 438 of SEQ ID NO:37 or a combination thereof, and wherein the OXOX variant polypeptide has OXOX activity;
(c) an isolated polynucleotide that encodes any of the polypeptides set forth in SEQ ID NOS: 21, 22, 23, 24, 25, 26, 27, 28, 29, 30, 31, 32, 33, 34, 66, 67, 68, 69, 70, 71, 72, 73, 74, 75, 76, 77, 78, 79, 80, 81, 82, 83, 84, 85, 86, 87, 88, 89, 90, 91, 92, 100, 101, 102, 103, 104, 105, or 106;
(d) an isolated polynucleotide comprising any of the sequences of SEQ ID NOS: 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 58, 59, 60, 61, 62, 63, 64, 65, 93, 94, 95, 96, 97, 98, or 99;
(e) an isolated polynucleotide comprising at least 30 nucleotides in length which hybridizes under stringent conditions to a polynucleotide of (a), (b), (c) or (d) wherein the conditions include hybridization in 40 to 45% formamide, 1 M NaCl, 1% SDS at 37° C. and a wash in 0.5× to 1×SSC at 55 to 60° C.;
(f) an isolated polynucleotide having at least 80%, 85%, 90% or 95% sequence identity to any of the sequences of SEQ ID NOS: 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 58, 59, 660, 61, 62, 63, 64, 65, 93, 94, 95, 96, 97, 98, or 99; wherein the % sequence identity is based on the entire encoding region and is determined by BLAST 2.0 under default parameters;
(g) an isolated polynucleotide amplified from a nucleic acid library using based on any of the sequences of SEQ ID NOS: 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 58, 59, 60, 61, 62, 63, 64, 65, 93, 94, 95, 96, 97, 98, or 99;
(h) a polynucleotide encoding a polypeptide that is at least 85%, 90%, or 95% identical to a polypeptide comprising the sequence set forth in SEQ ID NO: 21, 22, 23, 24, 25, 26, 27, 28, 29, 30, 31, 32, 33, 34, 66, 67, 68, 69, 70, 71, 72, 73, 74, 75, 76, 77, 78, 79, 80, 81, 82, 83, 84, 85, 86, 87, 88, 89, 90, 91, 92, 100, 101, 102, 103, 104, 105, or 106; wherein the encoded polypeptide has OXOX activity;
(i) a polynucleotide encoding a polypeptide fragment of at least about 200 amino acid residues from any of the polypeptides comprising the sequence set forth in SEQ ID NO: 21, 22, 23, 24, 25, 26, 27, 28, 29, 30, 31, 32, 33, 34, 66, 67, 68, 69, 70, 71, 72, 73, 74, 75, 76, 77, 78, 79, 80, 81, 82, 83, 84, 85, 86, 87, 88, 89, 90, 91, 92, 100, 101, 102, 103, 104, 105, or 106, wherein the encoded polypeptide fragment has OXOX activity;
(j) an isolated polynucleotide degenerate from any of (a) to (i) as a result of the genetic code; and
(k) a polynucleotide complementary to a polynucleotide of any one of (a) to (j).

2. The isolated or recombinant nucleic acid of claim 1, wherein the variant polynucleotide encodes an OXOX variant polypeptide that is selected from the group consisting of:
(i) an OXOX variant polypeptide that increases in a plant transgenic for the OXOX variant polypeptide the plant's resistance to a pathogen relative to a control plant that does not contain the polynucleotide of claim 1;
(ii) an OXOX variant polypeptide that has maintained or increased OXOX activity compared to the activity of a wild type OXOX; and
(iii) an OXOX variant polypeptide that has maintained or increased OXOX activity compared to the activity of a wild type OXOX, the OXOX variant polypeptide has maintained or increased digestibility as compared to the digestibility of a wild type OXOX enzyme.

3. The isolated or recombinant nucleic acid of claim 1, wherein at least one glycosylation site of the OXOX variant polypeptide or fragment thereof has been eliminated.

4. An expression cassette comprising at least one polynucleotide of claim 1 operably linked to a promoter.

5. A non-human host cell comprising at least one expression cassette of claim 4.

6. The host cell of claim 5, wherein the host cell is a plant cell.

7. A transgenic plant comprising stably incorporated in its genome an expression cassette comprising an OXOX variant polynucleotide of claim 1 operably linked to a promoter that drives expression in a plant cell.

8. The transgenic plant of claim 7, wherein the plant is a rice, wheat, peanut, sugarcane, sorghum, corn, cotton, soybean, vegetable, ornamental, conifer, alfalfa, spinach, tobacco, tomato, potato, sunflower, canola, barley or millet Brassica sp., safflower, sweet potato, cassava, coffee, coconut, pineapple, citrus trees, cocoa, tea, banana, palm, avocado, fig, guava, mango, olive, papaya, cashew, macadamia, almond, sugar beet, sugarcane, buckwheat, triticale, spelt, linseed, sugar cane, oil seed rape, canola, cress, Arabidopsis, cabbages, soya, pea, beans, eggplant, bell pepper, Tagetes, lettuce, Calendula, melon, pumpkin, squash, or oat plant.

9. A transgenic seed from the transgenic plant of claim 7.

10. An isolated or recombinant OXOX variant polypeptide selected from the group consisting of:
(a) an isolated or recombinant OXOX variant polypeptide comprising an amino acid sequence that has been substituted and wherein the amino acid substitution is least one amino acid substitution at a position that that corresponds to position 10, 19, 23, 26, 29, 35, 36, 38, 39, 40, 53, 54, 57, 58, 60, 61, 62, 63, 65, 68, 72, 79, 81, 83, 99, 102, 107, 115, 118, 124, 127, 131, 144, 148, 154, 159, 164, 166, 171, 174, 177, 181, 190, 192, 196, 200, 202, 203, 218, 219, 245, 259, 269, 278, 282, 287, 289, 290, 339, 349, 353, 359, 363, 373, 384, 387, 394, 395, 396, 399, 410, 425, 426, 427, 430, 433 or 436 of SEQ ID NO: 37, or an additional amino acid residue at position 437 or 438 of SEQ ID NO:37 or a combination thereof, and wherein the OXOX variant polypeptide has OXOX activity;
(b) an isolated or recombinant OXOX variant polypeptide having OXOX activity and wherein said polypeptide is encoded by any of the polynucleotides set forth in SEQ ID NOS: 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 58, 59, 60, 61, 62, 63, 64, 65, 93, 94, 95, 96, 97, 98, or 99 and wherein the OXOX variant polypeptide has OXOX activity; and
(c) an isolated or recombinant OXOX variant polypeptide having OXOX activity and is at least 80% identical to any of the sequences of SEQ ID NO: 21, 22, 23, 24, 25, 26, 27, 28, 29, 30, 31, 32, 33, 34, 66, 67, 68, 69, 70, 71, 72, 73, 74, 75, 76, 77, 78, 79, 80, 81, 82, 83, 84, 85, 86, 87, 88, 89, 90, 91, 92, 100, 101, 102, 103, 104, 105, or 106.

11. The isolated or recombinant OXOX variant polypeptide of claim 10, wherein the OXOX variant polypeptide has maintained or increased OXOX activity compared to the activity of a wild type OXOX.

12. The isolated or recombinant OXOX variant polypeptide or fragment thereof of claim 10, wherein the OXOX variant polypeptide or fragment thereof comprises at least one modification selected from the group consisting of:
(i) at least one of the glycosylation sites in the OXOX variant at position 60, 384, and 430 of SEQ ID NO:37 has been eliminated by an amino acid substitution at position 60, 384, and/or 430;
(ii) the amino acid residue at position 60 of SEQ ID NO:37 has been substituted with an isoleucine, the amino acid residue at position 384 of SEQ ID NO:37 has been substituted with a glutamine or valine, or the amino acid residue at position 430 of SEQ ID NO:37 has been substituted with a glutamine or aspartic acid; and
(iii) a threonine or valine substitution at position 10, a histidine substitution at position 19, a proline substitution at position 23, a serine at position 26, a glutamic acid substitution at position 35, a proline substitution at position 36, a glutamic acid substitution at position 38, an alanine at position 39, an alanine at position 40, a glycine or an asparagine substitution at position 53, a methionine substitution at position 54, a glutamine substitution at position 57, an aspartic acid substitution at position 58, an isoleucine, serine, arginine or glutamic acid substitution at position 60, a valine substitution at position 61, a valine substitution at position 63, an alanine or glutamine substitution at position 63, a glutamine substitution at position 65, a proline substitution at position 68, a glutamic acid substitution at position 72, an isoleucine substitution at position 79, a leucine substitution at position 81, a isoleucine substitution at position 83, a valine substitution at position 99, a alanine substitution at position 102, a serine substitution at position 107, a threonine substitution at position 115, a tyrosine substitution at position 118, an asparagine substitution at position 124, a leucine substitution at position 127, a threonine substitution at position 131, a threonine substitution at position 144, a methionine substitution at position 148, an alanine or glutamine substitution at position 154, a valine substitution at position 159, an aspartic acid substitution at position 164, a valine substitution at position 166, a glutamic acid substitution at position 171, a lysine substitution at position 174, a methionine substitution at position 177, a serine or glycine substitution at position 181, a proline substitution at position 190, an isoleucine or valine substitution at position 192, an isoleucine substitution at position 196, an asparagine substitution at position 200, an alanine substitution at position 202, an aspartic acid substitution at position 203, an asparagine substitution at position 218, an alanine substitution at position 219, a threonine substitution at position 245, a valine or tyrosine substitution at position 259, a glutamine substitution at position 269, a valine substitution at position 278, a phenylalanine substitution at position 282, a cysteine at position 287, an alanine substitution at position 289, a valine substitution at position 290, a tyrosine or valine substitution at position 339, a leucine substitution at position 349, a glutamic acid substitution at position 353, a phenylalanine substitution at position 359, an alanine substitution at position 363, a tyrosine substitution at position 373, a glutamine or valine substitution at position 384, a tyrosine substitution at position 387, an aspartic acid substitution at position 394, a valine substitution at position 395, a tyrosine substitution at position 396, an isoleucine substitution at position 399, an arginine substitution at position 410, an aspartic acid substitution at position 425, a serine substitution at position 426, a phenylalanine substitution at position 427, an aspartic acid or glutamine substitution at position 430, a leucine or serine substitution at position 433, an alanine substitution at position 436, a serine addition at position 437, or an aspartic acid addition at position 438 of SEQ ID NO: 37 or a combination thereof.

13. A method of modulating the level of oxalate oxidase (OXOX) protein in a plant or plant cell, comprising:
(a) transforming a plant cell with an OXOX variant polynucleotide comprising a member selected from the group consisting of:
(i) an isolated oxalate oxidase polynucleotide comprising a nucleotide that has been substituted and wherein the nucleotide substitution is one or more of the substitutions shown in FIG. 5;
(ii) an isolated OXOX variant polynucleotide encoding a polypeptide comprising an amino acid sequence that has been substituted and wherein the amino acid substitution is least one amino acid substitution at a position that corresponds to position 10, 19, 23, 26, 29, 35, 36, 38, 39, 40, 53, 54, 57, 58, 60, 61, 62, 63, 65, 68, 72, 79, 81, 83, 99, 102, 107, 115, 118, 124, 127, 131, 144, 148, 154, 159, 164, 166, 171, 174, 177, 181, 190, 192, 196, 200, 202, 203, 218, 219, 245, 259, 269, 278, 282, 287, 289, 290, 339, 349, 353, 359, 363, 373, 384, 387, 394, 395, 396, 399, 410, 425, 426, 427, 430, 433 or 436 of SEQ ID NO: 37, or an additional amino acid residue at position 437 or 438 of SEQ ID NO:37, or a combination thereof, and wherein the OXOX variant polypeptide has OXOX activity;
(iii) an isolated polynucleotide that encodes any of the polypeptides set forth in SEQ ID NOS: 18, 19, 21, 22, 23, 24, 25, 26, 27, 28, 29, 30, 31, 32, 33, 34, 35, 36, 37, 66, 67, 68, 69, 70, 71, 72, 73, 74, 75, 76, 77, 78, 79, 80, 81, 82, 83, 84, 85, 86, 87, 88, 89, 90, 91, 92, 100, 101, 102, 103, 104, 105, or 106;
(iv) an isolated polynucleotide comprising any of the sequences of SEQ ID NOS: 1, 2, 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 59, 60, 61, 62, 63, 64, 65, 93, 94, 95, 96, 97, 98, or 99;
(v) an isolated polynucleotide comprising at least 30 nucleotides in length which hybridizes under stringent conditions to a polynucleotide of (i), (ii), (iii) or (v) wherein the conditions include hybridization in 40 to 45% formamide, 1 M NaCl, 1% SDS at 37° C. and a wash in 0.5× to 1×SSC at 55 to 60° C.;
(vi) an isolated polynucleotide having at least 80% sequence identity to any of the sequences of SEQ ID NOS: 1, 2, 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 59, 60, 61, 62, 63, 64, 65, 93, 94, 95, 96, 97, 98, or 99, wherein the % sequence identity is based on the entire encoding region and is determined by BLAST 2.0 under default parameters;
(vii) an isolated polynucleotide amplified from a nucleic acid library using based on any of the sequences of SEQ ID NOS: 1, 2, 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 59, 60, 61, 62, 63, 64, 65, 93, 94, 95, 96, 97, 98, or 99;
(viii) an isolated polynucleotide degenerate from any of (i) to (vii) as a result of the genetic code; and
(ix) an isolated polynucleotide complementary to a polynucleotide of any one of (i) to (viii), wherein the polynucleotide is operably linked to a promoter, wherein the polynucleotide is in sense or antisense orientation; and optionally
(b) regenerating the transformed plant cell into a transformed plant that expresses the OXOX variant polynucleotide in an amount sufficient to modulate the level of OXOX protein in the plant;
wherein the level of the OXOX protein is increased or decreased in the plant or plant cell.

14. A method for increasing a plant's resistance to a pathogen, said method comprising the steps of:
(a) introducing into plant cells a construct comprising a fungal OXOX polynucleotide encoding a fungal OXOX polypeptide operably linked to a promoter functional in plant cells to yield transformed plant cells,
(b) regenerating a transgenic plant from said transformed plant cells, wherein said fungal OXOX when expressed in the cells of said transgenic plant at levels sufficient to increase a plant's resistance to the pathogen in said transgenic plant as compared to a control plant, wherein the control plant has not been transformed with the polynucleotide encoding the fungal OXOX.

15. The method of claim 14, wherein the pathogen is an oxalate producing fungus.

16. The method of claim 14, wherein the pathogen is from the genus of Sclerotinia.

17. The method of claim 14, wherein the fungal OXOX polynucleotide encoding a fungal OXOX polypeptide is from an oxalate producing fungus.

18. The method of claim 17, wherein the fungus is from the genus of Sclerotinia.

19. The method of claim 14, wherein fungal OXOX polynucleotide an OXOX variant polynucleotide selected from the group consisting of:
(i) an isolated oxalate oxidase polynucleotide comprising a nucleotide that has been substituted and wherein the nucleotide substitution is one or more of the substitutions shown in FIG. 5;
(ii) an isolated OXOX variant polynucleotide encoding a polypeptide comprising an amino acid sequence that has been substituted and wherein the amino acid substitution is least one amino acid substitution at a position that corresponds to position 10, 19, 23, 26, 29, 35, 36, 38, 39, 40, 53, 54, 57, 58, 60, 61, 62, 63, 65, 68, 72, 79, 81, 83, 99, 102, 107, 115, 118, 124, 127, 131, 144, 148, 154, 159, 164, 166, 171, 174, 177, 181, 190, 192, 196, 200, 202, 203, 218, 219, 245, 259, 269, 278, 282, 287, 289, 290, 339, 349, 353, 359, 363, 373, 384, 387, 394, 395, 396, 399, 410, 425, 426, 427, 430, 433 or 436 of SEQ ID NO: 37, or an additional amino acid residue at position 437 or 438 of SEQ ID NO:37, or a combination thereof, and wherein the OXOX variant polypeptide has OXOX activity;
(iii) an isolated polynucleotide that encodes any of the polypeptides set forth in SEQ ID NOS: 21, 22, 23, 24, 25, 26, 27, 28, 29, 30, 31, 32, 33, 34, 35, 36, 37, 66, 67, 68, 69, 70, 71, 72, 73, 74, 75, 76, 77, 78, 79, 80, 81, 82, 83, 84, 85, 86, 87, 88, 89, 90, 91, 92, 100, 101, 102, 103, 104, 105, or 106;
(iv) an isolated polynucleotide comprising any of the sequences of SEQ ID NOS: 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 59, 60, 61, 62, 63, 64, or 65;
(v) an isolated polynucleotide comprising at least 30 nucleotides in length which hybridizes under stringent conditions to a polynucleotide of (i), (ii), (iii) or (iv) wherein the conditions include hybridization in 40 to 45% formamide, 1 M NaCl, 1% SDS at 37° C. and a wash in 0.5× to 1×SSC at 55 to 60° C.;
(vi) an isolated polynucleotide having at least 80% sequence identity to any of the sequences of SEQ ID NOS: 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 59, 60, 61, 62, 63, 64, 65, 93, 94, 95, 96, 97, 98, or 99, wherein the % sequence identity is based on the entire encoding region and is determined by BLAST 2.0 under default parameters;
(vii) an isolated polynucleotide amplified from a nucleic acid library using based on any of the sequences of SEQ ID NOS: 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 59, 60, 61, 62, 63, 64, 65, 93, 94, 95, 96, 97, 98, or 99;
(viii) an isolated polynucleotide degenerate from any of (1) to (vi) as a result of the genetic code; and
(ix) an isolated polynucleotide complementary to a polynucleotide of any one of (i) to (viii).

20. The method of claim 14, wherein the promoter is a constitutive promoter or a pathogen-inducible promoter.

21. A pathogen-resistant plant produced by the method of claim 14.

22. A method for identifying OXOX variants with maintained or increased OXOX activity comprising:
(a) modifying OXOX polynucleotides to encode an OXOX variant polypeptide, wherein at least one of the OXOX polynucleotides used has at least 70% identity to a polynucleotide of SEQ ID NO: 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 59, 60, 61, 62, 63, 64, 65, 93, 94, 95, 96, 97, 98, or 99, and wherein the encoded OXOX variant polypeptide lacks one or more glycosylation sites,
(b) transforming the modified OXOX polynucleotide into a host cell; and
(c) selecting the host cells comprising OXOX variant polypeptides that have increased OXOX activity relative to a host cell that is not transformed with the OXOX variant polynucleotide.

23. The method of claim 22, further comprising eliminating at least one potential glycosylation site of the OXOX variant polypeptide.

24. The method of claim 22, wherein the OXOX variant lacks at least one of the glycosylation sites at position 60, 384 or 430 of SEQ ID NO: 37.

25. The method of claim 22, wherein the OXOX variant polypeptide or fragment thereof, wherein the amino acid residue at position 60 of SEQ ID NO:37 has been substituted with an isoleucine, the amino acid residue at position 384 of SEQ ID NO:37 has been substituted with a glutamine or valine, or the amino acid residue at position 430 of SEQ ID NO:37 has been substituted with a glutamine or aspartic acid.

26. The method of claim 22, wherein the host cell is a plant cell, the method further comprising subjecting the OXOX variants to Sclerotinia wherein the decreased area of lesions in a leaf expressing an OXOX variant relative to the area of lesions in a control leaf that is not transformed with the OXOX variant indicates that the OXOX variant has increased OXOX activity.

27. The method of claim 22, wherein the OXOX activity is increasing a plant's resistance to a pathogen as compared to a plant's resistance to the pathogen conferred by a wild type OXOX enzyme.

28. The method of claim 27, wherein the pathogen is an oxalate producing fungus.

29. The method of claim 28, wherein the pathogen is from the genus of Sclerotinia.

30. The method of claim 22, wherein the OXOX activity is increasing digestibility of the OXOX variant polypeptide encoded by the OXOX variant polynucleotide as compared to the digestibility of a wild type OXOX enzyme.

31. A method of generating a plant having increased resistance to a pathogen comprising:
(a) identifying a plant that has an oxalate oxidase (OXOX) gene allele that encodes an OXOX variant polypeptide selected from the group consisting of:
(i) an isolated or recombinant OXOX variant polypeptide comprising an amino acid sequence that has been substituted and wherein the amino acid substitution is least one amino acid substitution at a position that that corresponds to position 10, 19, 23, 26, 29, 35, 36, 38, 39, 40, 53, 54, 57, 58, 60, 61, 62, 63, 65, 68, 72, 79, 81, 83, 99, 102, 107, 115, 118, 124, 127, 131, 144, 148, 154, 159, 164, 166, 171, 174, 177, 181, 190, 192, 196, 200, 202, 203, 218, 219, 245, 259, 269, 278, 282, 287, 289, 290, 339, 349, 353, 359, 363, 373, 384, 387, 394, 395, 396, 399, 410, 425, 426, 427, 430, 433 or 436 of SEQ ID NO: 37, or an additional amino acid residue at position 437 or 438 of SEQ ID NO:37, or a combination thereof, and wherein the OXOX variant polypeptide has OXOX activity; and
(ii) an isolated or recombinant OXOX variant polypeptide having OXOX activity and wherein said polypeptide is encoded by any of the polynucleotides set forth in SEQ ID NOS: 4, 5, 6, 7, 8, 9, 10, 11, 12, 13, 14, 15, 16, 17, 39, 40, 41, 42, 43, 44, 45, 46, 47, 48, 49, 50, 51, 52, 53, 54, 55, 56, 57, 58, 59, 60, 61, 62, 63, 64, 65, 93, 94, 95, 96, 97, 98, or 99, and wherein the OXOX variant polypeptide has OXOX activity; and
(iii) an isolated or recombinant OXOX variant polypeptide having OXOX activity and is at least 80% identical to any of the sequences of SEQ ID NO: 21, 22, 23, 24, 25, 26, 27, 28, 29, 30, 31, 32, 33, 34, 66, 67, 68, 69, 70, 71, 72, 73, 74, 75, 76, 77, 78, 79, 80, 81, 82, 83, 84, 85, 86, 87, 88, 89, 90, 91, 92, 100, 101, 102, 103, 104, 105, or 106; wherein expression of the OXOX variant polypeptide results in increased pathogen resistance to the pathogen compared to plants lacking the allele; and
(b) generating progeny of said identified plant, wherein the generated progeny inherit the allele and have the increased pathogen resistance phenotype.
